(19) United States
(12) Patent Application Publication
(10) Pub. No.: US 2025/0019641 A1
(43) Pub. Date: Jan. 16, 2025

(54) COMPOSITIONS AND METHODS FOR PRODUCING FERMENTED DAIRY PRODUCTS FOR STORAGE AT AMBIENT TEMPERATURE

(71) Applicant: INTERNATIONAL N&H DENMARK APS, Kongens Lyngby (DK)

(72) Inventor: Yuejian Mao, Shanghai (CN)

(21) Appl. No.: 18/708,094

(22) PCT Filed: Nov. 2, 2022
(86) PCT No.: PCT/EP2022/080535

§ 371 Date: May 7, 2024

(30) Foreign Application Priority Data
Nov. 9, 2021 (WO) PCT/CN2021/129546

(51) Int. Cl. C12N 1/20; A23C 9/123; C12N 9/38; C12R 1/25; C12R 1/46

(52) U.S. Cl. CPC C12N 1/205; A23C 9/1234; C12N 9/2471; C12Y 302/01023; A23C 2220/202; C12N 2510/02; C12R 2001/25; C12R 2001/46

(57) ABSTRACT

Provided herein are compositions and methods for producing fermented dairy products that can be stored at ambient temperature. The compositions and methods provided herein include lactose-deficient bacteria capable of maintaining viability and/or pH when added to fermented dairy products having low levels of galactose and stored at ambient temperature. Also provided are fermented dairy products, e.g., yogurts, fermented dairy beverages, containing said lactose-deficient bacteria for storage at ambient temperature.

### FIELD OF THE INVENTION

Provided herein are compositions and methods for producing fermented dairy products that can be stored at ambient temperature. The compositions and methods provided herein include lactose-deficient bacteria capable of maintaining viability and/or pH when added to fermented dairy products having low levels of galactose and stored at ambient temperature. Also provided are fermented dairy products, e.g., yogurts, fermented dairy beverages, containing said lactose-deficient bacteria for storage at ambient temperature.

### BACKGROUND TO THE INVENTION

Fermented dairy products, such as yogurts and fermented milk beverages, capable of being stored at ambient temperature have grown in popularity with both consumers and manufacturers due to the ease with which the products can be stored and transported. However, existing methods for producing fermented dairy products for ambient storage include heat treatment (e.g., pasteurization) following fermentation, which greatly reduces or eliminates bacteria that may provide health benefits to consumers. Strategies to address this issue have included the addition of live bacteria to the food product following heat treatment.

The addition of live bacteria to the fermented food product, however, presents technical challenges. For example, the live bacteria may exhibit unwanted propagation in the food at ambient temperature, resulting in spoilage of the product. As another example, the live bacteria may not remain viable in the product due to an inability to survive in the product at ambient temperature.

Thus, there remains a need for compositions and methods for producing fermented dairy products containing live bacteria that can be stored at ambient temperature without negatively impacting the quality (e.g., healthy benefits, taste, texture) of the fermented dairy product. The compositions and methods provided herein address such needs.

### SUMMARY OF THE INVENTION

In a first aspect the present invention relates to a method of producing a fermented dairy product for storage at ambient temperature, comprising inoculating an initial fermented dairy product with one or more lactose-deficient bacterial strains, wherein the initial fermented food product comprises between about 0% and about 1% galactose.

In a second aspect the present invention relates to a culture or kit of parts comprising a lactose-deficient bacterial strain, wherein the lactose deficient bacterial strain has a lactose metabolic activity of about or less than about 50%, 40%, 30%, 20%, or 10% or no lactose metabolic activity compared to a lactose metabolic activity of a lactose-metabolizing bacteria comprising a beta-galactosidase protein having the amino acid sequence encoded by the sequence of nucleotides set forth by SEQ ID NO:1.

In a third aspect the present invention relates to a use of a culture of the present invention or kit of parts of the present invention to produce a fermented dairy product for storage at ambient temperature.

In a further aspect the present invention relates to a fermented dairy product for storage at ambient temperature obtained by the method or use of the invention.

In a further aspect the present invention relates to a fermented dairy product for storage at ambient temperature comprising a lactose-deficient bacterial strain, wherein the lactose deficient bacterial strain has a lactose metabolic activity of about or less than about 50%, 40%, 30%, 20%, or 10%, or no lactose metabolic activity compared to a lactose metabolic activity of a lactose-metabolizing bacteria comprising a beta-galactosidase protein having the amino acid sequence encoded by the sequence of nucleotides set forth by SEQ ID NO:1.

In a further aspect the present invention relates to a bacterial strain deposited at the DMSZ under accession number DSM33967 or a strain having all of the identifying characteristics of the L. plantarum strain deposited at DSMZ under accession number DSM33967.

In a further aspect the present invention relates to a method for manufacturing a lactose-deficient bacterial strain comprising:

a) providing a lactic acid bacterial strain as the mother strain, such as Lactiplantibacillus plantarum DSM 33121; b) introducing one or more mutations in the sequence of nucleotides of the promoter region of the beta-galactosidase gene lacL/M (PlacLM), such as the promoter region defined by any one of SEQ ID NO:10 or SEQ ID NO:11 of the strain; and c) screening for a lactose-deficient bacterial strain have a reduced or complete inability to metabolize lactose.

Each of the aspects and embodiments described herein are capable of being used together, unless excluded either explicitly or clearly from the context of the embodiment or aspect.

### BRIEF DESCRIPTION OF THE FIGURES

FIGS. 1A-1B are graphs showing the changes in pH (FIG. 1A) and bacterial viability (log CFU) (FIG. 1B) in yogurt fermented with an exemplary S. thermophilus (DSM32823) and inoculated with either an exemplary lactose-deficient L. plantarum (DSM33967) or an exemplary L. rhamnosus (ATCC53103) or commercial yogurt inoculated with an exemplary lactose-deficient L. plantarum (DSM33967) and stored for 150 days at ambient temperature.

### DETAILED DESCRIPTION OF THE INVENTION

Provided herein are compositions and methods for producing a fermented dairy product for storage at ambient temperature. The compositions and methods described herein circumvent known difficulties with maintaining the quality of fermented dairy products when stored at room temperature, thus addressing consumer and industry needs.

Consumer demand for fermented dairy products containing live bacteria (e.g., for their health benefits) that can be stored at ambient temperature continues to grow. Indeed, market trends demonstrate that consumers are looking for healthy food products, such as fermented dairy products like yogurts and fermented milk beverages, which are easy to consume, e.g., easy to be stored and transported. Fermented dairy products capable of being stored at ambient temperature are also advantageous in countries where the cold chain during distribution and storage of food products containing live bacteria is complex or challenging, and in some cases economically or technically impossible.

The production of fermented dairy products that can be stored at ambient temperature typically involves heat-treating the product after fermentation to kill or inhibit bacterial, e.g., starter culture, growth. Without being bound by theory, this step may be important for extending the shelf-life of the product at ambient temperature by preventing (1) bacterial-driven post-acidification capable of spoiling the fermented food product and/or (2) growth of unwanted harmful bacteria. However, since the heat treatment kills or inhibits growth of all bacteria, the consumer is left with a food product that lacks desirable health benefits potentially provided by viable beneficial bacteria, e.g., probiotics. Strategies to solve this issue have included the addition of live bacteria, e.g., lactic acid bacteria, to the fermented food product following heat treatment.

The addition of live bacteria to fermented dairy products following heat treatment is not necessarily a straightforward process since the bacteria have the potential to negatively impact the product, e.g., when stored at ambient temperature. For example, added live bacteria may multiply during storage of the product at ambient temperature, resulting in the production of undesired metabolites that impact the quality of the food product. By way of example, lactic acid bacteria are able to produce lactic acid at ambient temperature resulting in an unacceptable decrease in pH of the fermented food product, e.g., fermented dairy product. There is also the potential for the added live bacteria to die as a result of being unable to survive in the food matrix at ambient temperature, resulting in the loss of associated health benefits. Thus, there remains a need for compositions and methods capable of producing a fermented dairy product for storage at ambient temperature that contains viable bacteria without negatively impacting the product's quality (e.g., taste, texture, health benefits).

As described herein, it was surprisingly found that inoculating previously heat-treated fermented dairy products having low levels of galactose with live lactic acid bacteria incapable of fermenting lactose produced a fermented dairy product that could be stored at ambient temperature, e.g., room temperature, and maintain levels of viable bacteria and PH levels similar to those observed at the time of inoculation (see, Examples). The compositions and methods provided herein harness these surprising findings to produce a fermented dairy product containing live bacteria that can be stored at ambient temperature without negatively impacting the quality (e.g., health benefits, taste, texture) of the product.

In aspects of the invention are provided lactose-deficient bacteria and bacterial compositions, e.g., cultures, containing lactose-deficient bacteria that can be added to a fermented dairy product, e.g., an initial fermented dairy product as described herein, to produce a fermented dairy product for storage at ambient temperature. In some embodiments, the lactose-deficient bacteria are incapable of or have a reduced capacity to metabolize lactose. In some embodiments, the lactose-deficient bacteria are lactic acid bacteria (LABs). In some embodiments, the lactose-deficient bacteria are of the genus Lactobacillus. In some embodiments, the lactose-deficient bacteria are of the species Lactiplantibacillus plantarum. In some embodiments, the lactose-deficient bacteria are contained in a bacterial composition, e.g., culture. In some embodiments, the bacterial composition, e.g., culture, contains at least two, e.g., 3, 4, 5, 6, 7, or more bacterial strains including a lactose-deficient bacterial strain.

Also provided are methods for producing a fermented dairy product for storage at ambient temperature. In some embodiments, the methods include inoculating an initial fermented dairy product with lactose-deficient bacterial strain, e.g., a lactose-deficient Lactiplantibacillus plantarum strain, or culture containing a lactose-deficient bacterial strain, e.g., a lactose-deficient Lactiplantibacillus plantarum strain. In some embodiments, the initial fermented dairy product has a low level of galactose. For example, in some cases, the low level of galactose is a level of less than 2% per weight, e.g., less than 1%, 0.75%, 0.5%, 0.25%, or the level is 0%, of the initial fermented dairy product. In some embodiments, the initial fermented dairy product is produced by fermenting a milk substrate with a starter culture including a galactose-metabolizing bacterial strain, e.g., a galactose-metabolizing Streptococcus thermophilus, described herein. Bacteria capable of metabolizing galactose are referred to herein as galactose-metabolizing bacteria.

Further provided are kits of parts useful for producing a fermented dairy product for storage at ambient temperature. In some embodiments, the kits of parts include the lactose-deficient bacterial strain provided herein. In some embodiments, the kits of parts include a bacterial composition, e.g., culture, containing the lactose-deficient bacterial strain provided herein and at least one other, e.g., 1, 2, 3, 4, 5, or more bacterial strains.

In some embodiments, the kits of parts further include a bacteria useful for producing an initial fermented dairy product, e.g., a fermented dairy product that can be inoculated with a lactose-deficient bacterial strain. As described herein, in some cases, the initial fermented dairy product contains low levels of galactose. Thus, in some embodiments, the bacteria useful for producing an initial fermented dairy product is a bacteria capable of metabolizing galactose. In some embodiments, the galactose-metabolizing bacteria is of the genus Streptococcus. In some embodiments, the galactose-metabolizing bacteria is of the species Streptococcus thermophilus.

In aspects of the invention are provided fermented dairy products for storage at ambient temperature. The fermented dairy product for storage at ambient temperature may be stored at ambient temperature without the risk of negatively impacting the quality, e.g., health benefits, taste, texture, of the fermented dairy product. In some cases, the fermented dairy product for storage at ambient temperature can be stored at ambient temperature and maintain a level of viable bacteria, e.g., inoculated lactose-deficient bacterial strain as described herein, similar to inoculation levels. In some embodiments, the fermented dairy product for storage at ambient temperature can be stored at ambient temperature and maintain a pH similar to the pH reached at the end of fermentation. In some embodiments, the fermented dairy product for storage at ambient temperature can be stored at ambient temperature and maintain a pH similar to the pH at the time of inoculation with the lactose-deficient bacterial strain. In some embodiments, the fermented dairy product for storage at ambient temperature can be stored at ambient temperature and maintain a level of viable bacteria, e.g., inoculated lactose-deficient bacterial strain as described herein, similar to inoculation levels, and maintain a pH similar to the pH reached at the end of fermentation and/or similar to the pH at the time of inoculation with the lactose-deficient bacterial strain.

The compositions and methods provided herein are useful for producing a fermented dairy product that does not deteriorate in quality during storage at ambient temperature. The bacteria and methods provided herein produce ambient temperature fermented dairy products that provide advantages to manufacturers in terms of ease of distribution, while also satisfying a consumer desire for readily consumable fermented dairy products having health benefits.

Unless defined otherwise, all technical and scientific terms used herein have the same meaning as commonly understood by one of ordinary skill in the art to which this disclosure belongs.

This disclosure is not limited by the exemplary methods and materials disclosed herein, and any methods and materials similar or equivalent to those described herein can be used in the practice or testing of embodiments of this disclosure.

The headings provided herein are not limitations of the various aspects or embodiments of this disclosure which can be had by reference to the specification as a whole. The section headings used herein are for organizational purposes only and are not to be construed as limiting the subject matter described. Any terms defined are more fully defined by reference to the specification as a whole.

Definitions of terms may appear throughout the specification. It is to be understood that this disclosure is not limited to particular embodiments described, as such may, of course, vary. It is also to be understood that the terminology used herein is for the purpose of describing particular embodiments only and is not intended to be limiting.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and grammatical variants thereof, are inclusive or open-ended and do not exclude additional, non-recited members, elements or method steps. The terms “comprising”, “comprises” and “comprised of”, “including”, “includes” or “containing”, “contains”, and grammatical variants thereof also include the term “consisting of”.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range.

Where the stated range includes one or both of the limits, ranges excluding either or both of those included limits are also included in this disclosure.

Values and ranges may be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context. All values and ranges may implicitly include the term “about.”

All publications, including patent documents, scientific articles and databases, referred to in this application are incorporated by reference in their entirety for all purposes to the same extent as if each individual publication were individually incorporated by reference. Nothing herein is to be construed as an admission that such publications constitute prior art. If a definition set forth herein is contrary to or otherwise inconsistent with a definition set forth in the patents, applications, published applications and other publications that are herein incorporated by reference, the definition set forth herein prevails over the definition that is incorporated herein by reference.

### I. Bacteria and Bacterial Compositions

Provided herein are bacteria that can be added to an initial fermented dairy product, e.g., as described in Section II-A, to produce a fermented dairy product for storage at ambient temperature that is not compromised in quality during storage at ambient temperature (see, e.g., Section II). In some embodiments, the bacteria described herein may survive in the fermented dairy product for storage at ambient temperature during storage at ambient temperature. For example, in some embodiments, the bacteria provided herein remain viable at levels similar to those at the time of inoculation into the fermented dairy product for storage at ambient temperature during storage at ambient temperature. In some embodiments, the bacteria provided herein do not further acidify or only minimally acidify, e.g., as described in Section II-B, the fermented dairy product for storage at ambient temperature during storage at ambient temperature. In some embodiments, the bacteria provided herein survive in and do not further acidify or minimally acidify the fermented dairy product for storage at ambient temperature during storage at ambient temperature.

### A. Lactose-Deficient Bacteria

In some embodiments, the bacteria provided herein are lactose-deficient bacterial strains. As referred to herein, “lactose-deficient bacterial strains” are bacterial strains having a reduced capacity or complete inability to metabolize lactose.

In bacteria, the lactose operon, also referred to as the lac operon, encodes proteins involved in the transport and metabolism of lactose. The wild type lac operon typically includes a la-cLM gene (such as SEQ ID NO:7 and 8) and a lacS gene (such as SEQ ID NO:9), which encode beta-galactosidase, and lactose-and galactose-permease, respectively. It is contemplated herein that changes, e.g., mutations, in any one or more of the proteins encoded by the lac operon may result in or contribute to lactose deficiency of a bacterium. It is also contemplated that changes, e.g., mutations, in lac operon promoters, operons, repressors, and/or repressor promoters may result in or contribute to lactose deficiency of a bacterium.

In some embodiments, lactose-deficient bacterial strain does not encode, does not express, or does not express a functional form of one or more proteins that participate in and/or are required for lactose metabolism. In some embodiments, the lactose-deficient bacterial strain includes mutations in the nucleic acid sequences encoding and/or controlling expression of one or more proteins that participate in and/or are required for lactose metabolism. In some embodiments, lactose-deficient bacterial strain has decreased expression of one or more proteins that participate in and/or are required for lactose metabolism. In some embodiments, lactose-deficient bacterial strain does not express functional forms of one or more proteins that participate in and/or are required for lactose metabolism. In some embodiments, lactose-deficient bacterial strain does not express one or more proteins that participate in and/or are required for lactose metabolism. Thus, the lactose-deficient bacterial strain provided herein are incapable of or have a decreased capacity to metabolize lactose.

In some embodiments, a lactose-deficient bacterial strain is determined, e.g., identified, using functional methods and/or structural analyses. In some embodiments, a lactose-deficient bacterial strain is determined using both functional methods and structural analyses. In some embodiments, a lactose-deficient bacterial strain is determined, e.g., identified, using functional methods and/or structural analysis and comparing results of such methods and analyses to results obtained from bacteria known to metabolize lactose, i.e., lactose-metabolizing bacteria, in the same methods or analyses. As used herein, “lactose-metabolizing bacteria” are bacteria able to metabolize lactose, for example as determined functionally or structurally, e.g., by growth assays and/or genomic analysis.

In some embodiments, the lactose-deficient bacterial strain is determined by a functional assay. For example, in some embodiments, the lactose-deficient bacterial strain is determined by assessing survival and/or growth, e.g., growth rate, when grown in the presence of a media including lactose as the only energy source. In some embodiments, bacteria that do not survive are determined to be a lactose-deficient bacterial strain. In some embodiments, bacteria that do not demonstrate growth are determined to be a lactose-deficient bacterial strain. In some embodiments, bacteria that have a survival or growth rate that is less than about 95%, 90%, 85%, 80%, 75%, 70%, 65%, 60%, 55%, 50%, 45%, 40%, 35%, 30%, 25%, 20%, 15%, 10%, or 5% of the survival or growth rate of a lactose-metabolizing bacteria are determined to be a lactose-deficient bacterial strain.

In some embodiments, the functional assay assesses, e.g., quantifies, lactose metabolic activity. Methods of assaying lactose metabolism are described by Test A as described in the example section.

In some embodiments, bacteria having no detectable lactose metabolic activity are determined to be a lactose-deficient bacterial strain. In some embodiments, bacteria having about or less than about 95%, 90%, 85%, 80%, 75%, 70%, 65%, 60%, 55%, 50%, 45%, 40%, 35%, 30%, 25%, 20%, 15%, 10%, or 5% compared to the lactose metabolic activity of a lactose-metabolizing bacteria are determined to be a lactose-deficient bacterial strain.

In some embodiments, the lactose-deficient bacterial strain, e.g., having reduced or no lactose metabolism, is determined by structural analysis. For example, DNA sequencing may be used to sequence nucleic acids encoding proteins that participate in and/or are required for lactose metabolism, and/or promoters, operons, repressors, and/or repressor promoters that control expression of proteins that participate in and/or are required for lactose metabolism. In some embodiments, the lac operon and/or portions of sequences contained in the lac operon of a bacteria are compared to the lac operon and/or portions of sequences contained in the lac operon contained in a lactose-metabolizing bacteria. Similarly, the amino acid sequence of proteins that participate in and/or are required for lactose metabolism may be assessed in a bacteria and compared to the amino acid sequence of proteins that participate in and/or are required for lactose metabolism expressed in a lactose-metabolizing bacteria. In some embodiments, the lactose-deficient bacterial strain contains nucleic acid sequences and/or amino acid sequences included in the lactose metabolic pathway that are mutated compared to the corresponding nucleic acid sequences and/or amino acid sequences of the lactose metabolic pathway contained in a lactose-metabolizing bacteria. Sequencing methods, including whole genome analysis, are known in the art as well as are methods for identifying mutations nucleic acid sequences and amino acid sequences.

In some embodiments, a wild type lac operon is or includes the sequence set forth by SEQ ID NO:1. In some embodiments, a bacterium containing a lac operon having or including the sequence set forth by SEQ ID NO:1 is a lactose-metabolizing bacterium.

In some embodiments, a lactose-deficient bacterial strain is determined by comparing the nucleic acid sequence of a lac operon of a bacteria to the sequence set forth by SEQ ID NO:1.

In some embodiments, if the lac operon of the bacteria has about or less than about 95%, 90%, 85%, 80%, 75%, 70%, 65%, 60%, 55%, 50%, 45%, 40%, 35%, 30%, 25%, 20%, 15%, 10%, or 5% sequence identity to the sequence set forth in SEQ ID NO:1, the bacteria is determined to be a lactose-deficient bacterial strain. In some embodiments, if the lac operon of the bacteria has about or less than about 75%, 70%, 65%, 60%, 55%, 50%, 45%, 40%, 35%, 30%, 25%, 20%, 15%, 10%, or 5% sequence identity to the sequence set forth in SEQ ID NO:1, the bacteria is determined to be a lactose-deficient bacterial strain. In some embodiments, if the lac operon of the bacteria has about or less than about 50% sequence identity to the sequence set forth in SEQ ID NO:1, the bacteria is determined to be a lactose-deficient bacterial strain.

In some embodiments, a lactose-deficient bacterial strain contains a lac operon nucleic acid sequence having about or less than about 95%, 90%, 85%, 80%, 75%, 70%, 65%, 60%, 55%, 50%, 45%, 40%, 35%, 30%, 25%, 20%, 15%, 10%, or 5% sequence identity to the sequence set forth in SEQ ID NO:1. In some embodiments, the lactose-deficient bacterial strain contains a lac operon nucleic acid sequence having about or less than about 75%, 70%, 65%, 60%, 55%, 50%, 45%, 40%, 35%, 30%, 25%, 20%, 15%, 10%, or 5% sequence identity to the sequence set forth in SEQ ID NO:1. In some embodiments, the lactose-deficient bacterial strain contains a lac operon nucleic acid sequence having about or less than about 50% sequence identity to the sequence set forth in SEQ ID NO:1, the bacteria is determined to be a lactose-deficient bacterial strain. In some embodiments, a lactose-deficient bacterial strain does not contain a lac operon.

In some embodiments, a lactose-deficient bacterial strain is determined via one or more functional assays. In some embodiments, the one or more functional assays assess, e.g., quantify, a bacteria's lactose metabolic activity. In some embodiments, the lactose metabolic activity of a bacteria is determined and compared to a bacteria containing a lac operon including the sequence set forth by SEQ ID NO:1. In some embodiments, if the lactose metabolic activity of the bacteria is about or less than about 95%, 90%, 85%, 80%, 75%, 70%, 65%, 60%, 55%, 50%, 45%, 40%, 35%, 30%, 25%, 20%, 15%, 10%, or 5% compared to the lactose metabolic activity of the bacteria containing a lac operon including the sequence set forth by SEQ ID NO:1, the bacteria is determined to be a lactose-deficient bacterial strain. In some embodiments, if the lactose metabolic activity of the bacteria is about or less than about 75%, 70%, 65%, 60%, 55%, 50%, 45%, 40%, 35%, 30%, 25%, 20%, 15%, 10%, or 5% compared to the lactose metabolic activity of the bacteria containing a lac operon including the sequence set forth by SEQ ID NO:1, the bacteria is determined to be a lactose-deficient bacterial strain. In some embodiments, if the lactose metabolic activity of the bacteria is about or less than about 50% compared to the lactose metabolic activity of the bacteria containing a lac operon including the sequence set forth by SEQ ID NO:1, the bacteria is determined to be a lactose-deficient bacterial strain.

In some embodiments, a lactose-deficient bacterial strain has a lactose metabolic activity of about or less than about 95%, 90%, 85%, 80%, 75%, 70%, 65%, 60%, 55%, 50%, 45%, 40%, 35%, 30%, 25%, 20%, 15%, 10%, or 5% compared to the lactose metabolic activity of the bacteria containing a lac operon including the sequence set forth by SEQ ID NO:1. In some embodiments, the lactose-deficient bacterial strain has a lactose metabolic activity of about or less than about 75%, 70%, 65%, 60%, 55%, 50%, 45%, 40%, 35%, 30%, 25%, 20%, 15%, 10%, or 5% compared to the lactose metabolic activity of the bacteria containing a lac operon including the sequence set forth by SEQ ID NO:1. In some embodiments, a lactose-deficient bacterial strain has a lactose metabolic activity of about or less than about 50% compared to the lactose metabolic activity of the bacteria containing a lac operon including the sequence set forth by SEQ ID NO:1. In some embodiments, a lactose-deficient bacterial strain has no detectable lactose metabolic activity compared to the lactose metabolic activity of the bacteria containing a lac operon including the sequence set forth by SEQ ID NO:1. In some embodiments, a lactose-deficient bacterial strain has no detectable lactose metabolic activity.

In some embodiments, the lactose-deficient bacterial strain is determined by structural analysis, e.g., sequencing and comparing its lac operon, and/or functional assay, e.g., quantification of lactose metabolic activity.

In some embodiments, a lactose-deficient bacterial strain does not encode, does not express, has reduced expression of, or expresses a non-functional form of at least a beta-galactosidase protein. In some embodiments, a lactose-deficient bacterial strain does not encode, does not express, has reduced expression of, or expresses a non-functional form of a beta-galactosidase protein. Beta-galactosidase is an intracellular enzyme that cleaves the disaccharide lactose into its components, glucose and galactose. A wild type beta-galactosidase protein may be encoded by nucleic acid sequences, e.g., lacLM genes, including the sequence set forth by SEQ ID NO:7 and 8. In some embodiments, bacteria contain nucleic acid sequences including the sequences set forth by SEQ ID NO: 7 and 8 are lactose-metabolizing bacteria.

In some embodiments, the lactose-deficient bacterial strain does not express or has reduced expression of a beta-galactosidase protein compared to an average expression level, for example a level of expression in a lactose-metabolizing bacterium. In some embodiments, the lactose-deficient bacterial strain contains a nucleic acid sequence encoding a beta-galactosidase (e.g., lacLM genes) that contains one or more mutations, for example, additions, substitutions, or deletions. In some embodiments, the one or more mutations may result in frame shifts, stop codons, and/or protein misfolding, resulting in a non-functional protein. In some embodiments, the beta-galactosidase protein is not expressed in a functional form. For example, the beta-galactosidase protein may be expressed as a truncated protein or a misfolded protein.

In some embodiments, the lactose-deficient bacterial strain may be identified by comparing the nucleic acid sequence encoding a beta-galactosidase protein of a bacteria to the sequence set forth by SEQ ID NO: 7 and 8. In some embodiments, if the nucleic acid sequence encoding the beta-galactosidase protein of the bacteria has about or less than about 100%, 98%, 95%, 90%, 85%, 80%, 75%, 70%, 65%, 60%, 55%, 50%, 45%, 40%, 35%, 30%, 25%, 20%, 15%, 10%, or 5% sequence identity to the sequence set forth in SEQ ID NO: 7 and 8,the bacteria is determined to be a lactose-deficient bacterial strain. In some embodiments, if the nucleic acid sequence encoding the beta-galactosidase protein of the bacteria has about or less than about 75%, 70%, 65%, 60%, 55%, 50%, 45%, 40%, 35%, 30%, 25%, 20%, 15%, 10%, or 5% sequence identity to the sequence set forth in SEQ ID NO: 7 and 8, the bacteria is determined to be a lactose-deficient bacterial strain. In some embodiments, if the nucleic acid sequence encoding the beta-galactosidase protein of the bacteria has about or less than about 50% sequence identity to the sequence set forth in SEQ ID NO: 7 and 8, the bacteria is determined to be a lactose-deficient bacterial strain.

In some embodiments, a lactose-deficient bacterial strain contain nucleic acid sequences encoding a beta-galactosidase protein having about or less than about 99%, 98%, 97%, 96%, 95%, 90%, 85%, 80%, 75%, 70%, 65%, 60%, 55%, 50%, 45%, 40%, 35%, 30%, 25%, 20%, 15%, 10%, or 5% sequence identity to the sequence set forth in SEQ ID NO: 7 and 8. In some embodiments, the lactose-deficient bacterial strain contains nucleic acid sequences encoding a beta-galactosidase protein having about or less than about 75%, 70%, 65%, 60%, 55%, 50%, 45%, 40%, 35%, 30%, 25%, 20%, 15%, 10%, or 5% sequence identity to the sequence set forth in SEQ ID NO: 7 and 8. In some embodiments, the lactose-deficient bacterial strain contains nucleic acid sequences encoding a beta-galactosidase protein having about or less than about 50% sequence identity to the sequence set forth in SEQ ID NO: 7 and 8. In some embodiments, the lactose-deficient bacterial strain does not contain a nucleic acid sequence encoding a beta-galactosidase protein.

In some embodiments, a lactose metabolic activity of a bacteria is determined and compared to a bacteria containing a beta-galactosidase protein encoded by the nucleic acid sequence set forth by SEQ ID NO: 7 and 8. In some embodiments, if the lactose metabolic activity of a bacteria is about or less than about 95%, 90%, 85%, 80%, 75%, 70%, 65%, 60%, 55%, 50%, 45%, 40%, 35%, 30%, 25%, 20%, 15%, 10%, or 5% compared to the lactose metabolic activity of a bacteria containing a beta-galactosidase protein encoded by the nucleic acid sequence set forth by SEQ ID NO: 7 and 8, the bacteria is determined to be a lactose-deficient bacterial strain. In some embodiments, if the lactose metabolic activity of a bacteria is about or less than about 75%, 70%, 65%, 60%, 55%, 50%, 45%, 40%, 35%, 30%, 25%, 20%, 15%, 10%, or 5% compared to the lactose metabolic activity of a bacteria containing a beta-galactosidase protein encoded by the nucleic acid sequence set forth by SEQ ID NO: 7 and 8, the bacteria is determined to be a lactose-deficient bacterial strain. In some embodiments, if the lactose metabolic activity of a bacteria is about or less than about 50% compared to the lactose metabolic activity of the bacteria containing a beta-galactosidase protein encoded by the nucleic acid sequence set forth by SEQ ID NO: 7 and 8, the bacteria is determined to be a lactose-deficient bacterial strain.

In some embodiments, a lactose-deficient bacterial strain has a lactose metabolic activity of about or less than about 95%, 90%, 85%, 80%, 75%, 70%, 65%, 60%, 55%, 50%, 45%, 40%, 35%, 30%, 25%, 20%, 15%, 10%, or 5% compared to a lactose metabolic activity of the bacteria containing a beta-galactosidase protein encoded by the nucleic acid sequence set forth by SEQ ID NO: 7 and 8. In some embodiments, the lactose-deficient bacterial strain has a lactose metabolic activity of about or less than about 75%, 70%, 65%, 60%, 55%, 50%, 45%, 40%, 35%, 30%, 25%, 20%, 15%, 10%, or 5% compared to the lactose metabolic activity of the bacteria containing a beta-galactosidase protein encoded by the nucleic acid sequence set forth by SEQ ID NO: 7 and 8. In some embodiments, the lactose-deficient bacterial strain has a lactose metabolic activity of about or less than about 50% compared to the lactose metabolic activity of the bacteria containing a beta-galactosidase protein encoded by the nucleic acid sequence set forth by SEQ ID NO: 7 and 8. In some embodiments, the lactose-deficient bacterial strain has no detectable lactose metabolic activity compared to the bacteria containing a beta-galactosidase protein encoded by the nucleic acid sequence set forth by SEQ ID NO: 7 and 8. In some embodiments, a lactose-deficient bacterial strain has no detectable lactose metabolic activity compared to the bacteria containing a beta-galactosidase protein encoded by the nucleic acid sequence set forth by SEQ ID NO: 7 and 8.

In some embodiments, the lactose-deficient bacterial strain is determined by structural analysis, e.g., comparing its nucleic acid sequence encoding a beta-galactosidase protein and/or its beta-galactosidase protein amino acid sequence, and/or functional assay. Methods of determining whether the beta-galactosidase protein of a bacteria is mutated and a nucleic acid sequence encoding a beta-galactosidase protein of a bacteria is mutated are known in the art. Methods of measuring lactose metabolic activity of a bacterium are generally known in the art, for example as described above.

In some embodiments, a lactose-deficient bacterial strain contains a promoter nucleic acid sequence of the beta-galactosidase genes that has one or more mutations compared to the nucleic acid sequence set forth by SEQ ID NO: 11. In some embodiments, the one or more mutations are deletions, insertions, and/or substitutions of one or more nucleotide of the nucleic acid sequence. In some embodiments, the one or more mutations are substitutions of one or more nucleotide, such as one or two nucleotides, of the nucleic acid sequence. In some embodiments, the one or more mutations, such as one or two, occur at any position in the sequence set forth by SEQ ID NO: 11. In some embodiments, the one or more mutations occur between positions 272 to 273 of the sequence set forth by SEQ ID NO: 11. In some embodiments, the lactose-deficient bacterial strain contains a nucleic acid sequence having the sequence set forth by SEQ ID NO:10. In some embodiments, the one or more mutations occur in the −10 region of the promoter, such as a two-nucleotide mutation GG to AA at position 272-273 of SEQ ID NO:11.

In some embodiments, the lactose-deficient bacterial strain is a lactic acid bacterial strain. In some embodiments, the lactic acid bacterial strain is a strain of a species of Lactiplantibacillus plantarum (also known as Lactobacillus plantarum), Lactobacillus zymae (also known as Levilactobacillus zymae), Lactobacillus rossiae (also known as Furfurilactobacillus rossiae), Lactobacillus collinoides (also known as Secundilactobacillus collinoides), Lactobacillus similis (also known as Secundilactobacillus similis), Lactobacillus versmoldensis (also known as Companilactobacillus versmoldensis), Lactobacillus acidipiscis (also known as Ligilactobacillus acidipiscis), Lactobacillus hammesii (also known as Levilactobacillus hammesii), Lactobacillus namurensis (also known as Levilactobacillus namurensis), Lactobacillus nodensis (also known as Companilactobacillus nodensis) and Lactobacillus tucceti (also known as Companilactobacillus tucceti);. In some embodiments, the lactic acid bacteria is a strain of species Lactiplantibacillus plantarum. In some embodiments, the lactic acid bacteria is a strain of species Lactobacillus zymae. I In some embodiments, the lactic acid bacteria is a strain of species Lactobacillus rossiae. In some embodiments, the lactic acid bacteria is a strain of species Lactobacillus collinoides. In some embodiments, the lactic acid bacteria is a strain of species Lactobacillus similis. In some embodiments, the lactic acid bacteria is a strain of species Lactobacillus versmoldensis. In some embodiments, the lactic acid bacteria is a strain of species Lactobacillus acidipiscis. In some embodiments, the lactic acid bacteria is a strain of species Lactobacillus hammesii. In some embodiments, the lactic acid bacteria is a strain of species Lactobacillus namurensis. In some embodiments, the lactic acid bacteria is a strain of species Lactobacillus nodensis. In some embodiments, the lactic acid bacteria is a strain of species Lactobacillus tucceti.

In some embodiments, the lactose-deficient bacterial strain is a lactose-deficient Lactiplantibacillus plantarum strain. In some embodiments, the lactose-deficient L. plantarum strain contains a lac operon having about or less than about 95%, 90%, 85%, 80%, 75%, 70%, 65%, 60%, 55%, 50%, 45%, 40%, 35%, 30%, 25%, 20%, 15%, 10%, or 5% sequence identity to the sequence set forth in SEQ ID NO:1. In some embodiments, the lactose-deficient L. plantarum strain contains a lac operon having about or less than about 75%, 70%, 65%, 60%, 55%, 50%, 45%, 40%, 35%, 30%, 25%, 20%, 15%, 10%, or 5% sequence identity to the sequence set forth in SEQ ID NO:1. In some embodiments, the lactose-deficient L. plantarum strain contains a lac operon having about or less than about 50%, 45%, 40%, 35%, 30%, 25%, 20%, 15%, 10%, or 5% sequence identity to the sequence set forth in SEQ

ID NO:1. In some embodiments, the lactose-deficient L. plantarum strain contains a lac operon having about or less than about 50% sequence identity to the sequence set forth in SEQ ID NO:1. In some embodiments, a lactose-deficient L. plantarum strain does not contain a lac operon.

In some embodiments, the lactose-deficient L. plantarum strain has a lactose metabolic activity of about or less than about 95%, 90%, 85%, 80%, 75%, 70%, 65%, 60%, 55%, 50%, 45%, 40%, 35%, 30%, 25%, 20%, 15%, 10%, or 5% compared to the lactose metabolic activity of an L. plantarum strain containing a lac operon including the sequence set forth by SEQ ID NO:1. In some embodiments, the lactose-deficient L. plantarum strain has a lactose metabolic activity of about or less than about 75%, 70%, 65%, 60%, 55%, 50%, 45%, 40%, 35%, 30%, 25%, 20%, 15%, 10%, or 5% compared to the lactose metabolic activity of an L. plantarum strain containing a lac operon including the sequence set forth by SEQ ID NO:1. In some embodiments, the lactose-deficient L. plantarum strain has a lactose metabolic activity of about or less than about 50%, 45%, 40%, 35%, 30%, 25%, 20%, 15%, 10%, or 5% compared to the lactose metabolic activity of an L. plantarum strain containing a lac operon including the sequence set forth by SEQ ID NO:1. In some embodiments, the lactose-deficient L. plantarum strain has a lactose metabolic activity of about or less than about 50% compared to the lactose metabolic activity of an L. plantarum strain containing a lac operon including the sequence set forth by SEQ ID NO:1. In some embodiments, the lactose-deficient L. plantarum strain has a lactose metabolic activity of about or less than about 25% compared to the lactose metabolic activity of an L. plantarum strain containing a lac operon including the sequence set forth by SEQ ID NO:1. In some embodiments, the lactose-deficient

L. plantarum strain has a lactose metabolic activity of about or less than about 15% compared to the lactose metabolic activity of an L. plantarum strain containing a lac operon including the sequence set forth by SEQ ID NO:1. In some embodiments, the lactose-deficient L. plantarum strain has a lactose metabolic activity of about or less than about 10% compared to the lactose metabolic activity of an L. plantarum strain containing a lac operon including the sequence set forth by SEQ ID NO:1. In some embodiments, the lactose-deficient L. plantarum strain has a lactose metabolic activity of about or less than about 25% compared to the lactose metabolic activity of an L. plantarum strain containing a lac operon including the sequence set forth by SEQ ID NO:1. In some embodiments, the lactose-deficient

L. plantarum strain has no detectable lactose metabolic activity compared to the lactose metabolic activity of an L. plantarum strain containing a lac operon including the sequence set forth by SEQ ID NO:1.

In some embodiments, the lactose-deficient L. plantarum strain contains a nucleic acid sequence encoding a beta-galactosidase protein having about or less than about 95%, 90%, 85%, 80%, 75%, 70%, 65%, 60%, 55%, 50%, 45%, 40%, 35%, 30%, 25%, 20%, 15%, 10%, or 5% sequence identity to the sequence set forth in SEQ ID NO:7 and 8. In some embodiments, the lactose-deficient L. plantarum strain contains nucleic acid sequences encoding a beta-galactosidase protein having about or less than about 75%, 70%, 65%, 60%, 55%, 50%, 45%, 40%, 35%, 30%, 25%, 20%, 15%, 10%, or 5% sequence identity to the sequence set forth in SEQ ID NO: 7 and 8. In some embodiments, the lactose-deficient L. plantarum strain contains nucleic acid sequences encoding a beta-galactosidase protein having about or less than about 50%, 45%, 40%, 35%, 30%, 25%, 20%, 15%, 10%, or 5% sequence identity to the sequence set forth in SEQ ID NO: 7 and 8. In some embodiments, the lactose-deficient L. plantarum strain contains nucleic acid sequences encoding a beta-galactosidase protein having about or less than about 50% sequence identity to the sequence set forth in SEQ ID NO: 7 and 8.

In some embodiments, the lactose-deficient L. plantarum strain has a lactose metabolic activity of about or less than about 95%, 90%, 85%, 80%, 75%, 70%, 65%, 60%, 55%, 50%, 45%, 40%, 35%, 30%, 25%, 20%, 15%, 10%, or 5% compared to the lactose metabolic activity of an L. plantarum strain including nucleic acid sequences encoding a beta-galactosidase protein set forth by SEQ ID NO: 7 and 8. In some embodiments, the lactose-deficient L. plantarum strain has a lactose metabolic activity of about or less than about 75%, 70%, 65%, 60%, 55%, 50%, 45%, 40%, 35%, 30%, 25%, 20%, 15%, 10%, or 5% compared to the lactose metabolic activity of an L. plantarum strain including nucleic acid sequences encoding a beta-galactosidase protein set forth by SEQ ID NO: 7 and 8. In some embodiments, the lactose-deficient L. plantarum strain has a lactose metabolic activity of about or less than about 50%, 45%, 40%, 35%, 30%, 25%, 20%, 15%, 10%, or 5% compared to the lactose metabolic activity of an L. plantarum strain including nucleic acid sequences encoding a beta-galactosidase set forth by SEQ ID NO: 7 and 8. In some embodiments, the lactose-deficient L. plantarum strain has a lactose metabolic activity of about or less than about 50% compared to the lactose metabolic activity of an L. plantarum strain including nucleic acid sequences encoding a beta-galactosidase set forth by SEQ ID NO: 7 and 8. In some embodiments, a lactose-deficient bacterial strain has no detectable lactose metabolic activity.

In some embodiments, the lactose-deficient L. plantarum strain has or includes any one or a combination of the genomic or metabolic activities described herein that determine a lactose-deficient bacterial strain.

In some embodiments, the lactose-deficient L. plantarum strain is the strain DGCC13484 that was deposited with the German Collection of Microorganisms and Cell Cultures (DSMZ) under accession no. DSM33967 or a mutant strain thereof. In some embodiments, the mutant strain is obtained by using the deposited strain as starting material. In some embodiments, the mutant is a strain having all of the identifying characteristics of the strain deposited at DSMZ.

As used herein, a mutant may be understood as a strain derived from a strain described herein by means of e.g., genetic engineering, radiation, UV light, and/or chemical treatment and/or methods that induce changes in the genome. The mutant may be a functionally equivalent mutant, e.g., a mutant that has substantially the same, or improved, properties (e.g., regarding lactose deficiency, post-acidification, and/or phage robustness) as the parent strain. In some embodiments, the mutant may demonstrate phage resistance and/or reduced phage sensitivity that is the same or different from the phage resistance and/or reduced phage sensitivity of the parent strain. In some embodiments, the phage resistance and/or reduced phage sensitivity may result from differences at one or more CRISPR loci compared to the parent strain. In some embodiments, the difference may be the addition of one or more spacers in one or more CRISPR loci. In some embodiments, the phage resistance and/or reduced phage sensitivity may result from non-CRISPR-mediated phage resistance mechanisms that differ from those of the parent strain. Thus, in some embodiments, the CRISPR loci of the mutant strain are identical to the CRISPR loci of the parent strain but the mutant strain displays different phage resistances and/or reduced phage sensitivities compared to the parent strain. Methods for inducing or generating phage resistance or reduced sensitivity and detecting such features are known in the art. Non-limiting methods for inducing phage resistance or reduced phage sensitivity may be found, for example, in published international applications WO 2007/025097, WO 2007/136815, and WO 2008/108989, which are incorporated by reference in their entirety. Such mutants are a part of the present invention.

A mutant, e.g., mutant strain, as referred to herein may be a strain obtained by subjecting a strain described herein to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood as containing one mutagenization step followed by a screening/selection step). In some embodiments, no more than 20, or no more than 10, or no more than 5 treatments (or screening/selection steps) are carried out to produce a mutant. In some embodiments, less than 5%, or less than 1% or less than 0.1% of the nucleotides in the bacterial genome have been shifted with another nucleotide, deleted, substituted or added compared to the mother strain. In some embodiments, the mutant contains no more than 20, in particular no more than 10, in particular no more than 5, in particular no more than 4, in particular no more than 3, in particular no more than 2, and in particular no more than 1 nucleotide mutation of the bacterial genome as compared to the mother strain, wherein a mutation is a substitution, an insertion or a deletion of a nucleotide. In some embodiments, a mutant is obtained by subjecting a strain described herein to a selective pressure or a stressor.

In some embodiments the mother strain is a Lactiplantibacillus plantarum DSM33121 deposited at the DSMZ on May 22, 2019 and a variant of the DSM33121 strain.

### B. Bacterial Composition

Also provided are bacterial compositions, also referred to herein as cultures, including a lactose-deficient bacterial strain as described in Section I-A above. In some embodiments, the culture includes a lactose-deficient bacterial strain. In some embodiments, the culture includes a lactose-deficient L. plantarum strain. In some embodiments, the culture includes the lactose-deficient L. plantarum strain deposited at the DSM33967 or a mutant thereof. In some embodiments, the culture includes a lactose-deficient L. plantarum strain having all of the identifying features of the lactose-deficient L. plantarum strain deposited at the DSM33967.

In some embodiments, the bacterial composition is a pure culture, i.e., comprises or consists of a single lactose-deficient bacterial strain of the invention. In some embodiments, the bacterial composition is a pure culture, i.e., comprises or consists of a single lactose-deficient Lactiplantibacillus plantarum strain of the invention. In some embodiments, the bacterial composition is a mixed culture, i.e., comprises or consists of a lactose-deficient bacterial strain of the invention and at least one other bacterial strain. In some embodiments, the bacterial composition is a mixed culture, i.e., comprises or consists of a lactose-deficient Lactiplantibacillus plantarum strain of the invention and at least one other bacterial strain. In some embodiments, the at least one other bacterial strain is a lactic acid bacterial strain. In some embodiments, the at least one other bacterial strain is a lactose-deficient bacterial strain, e.g., as described herein.

In some embodiments, the bacterial composition, either as a pure or mixed culture as defined above, further comprises a food acceptable ingredient.

In some embodiments, the bacterial composition, either as a pure or mixed culture as defined above is in a frozen, dried, freeze-dried, liquid or solid format, in the form of pellets or frozen pellets, or in a powder or dried powder. In some embodiments, the bacterial composition of the invention is in a frozen format or in the form of pellets or frozen pellets. In some embodiments, the pellets or frozen pellets are contained in one or more boxes or sachets. In some embodiments, the bacterial composition as defined herein is in a powder form, such as a dried or freeze-dried powder. In some embodiments, the powder, e.g., dried or freeze-dried powder, is contained in one or more boxes or sachets.

In some embodiments, the bacterial composition of the invention, either as a pure culture or mixed culture as defined above, and whatever the format (frozen, dried, freeze-dried, liquid or solid format, in the form of pellets or frozen pellets, or in a powder or dried powder) includes the lactose-deficient Lactiplantibacillus plantarum strain of the invention in a concentration in the range of about 10⁵ to about 10¹² cfu (colony forming units) per gram of the bacterial composition. In some embodiments, the concentration of the Lactiplantibacillus plantarum strain within the bacterial composition of the invention is in the range of 10⁷ to 10¹² cfu per gram of the bacterial composition, and in particular at least 10⁷, at least 10⁸, at least 10⁹, at least 10¹⁰ or at least 10¹¹ CFU/g of the bacterial composition. In some embodiments, when in the form of frozen or dried concentrate, the concentration of the lactose-deficient Lactiplantibacillus plantarum strain of the invention-as pure culture or as a mixed culture-within the bacterial composition is in the range of 10⁸ to 10¹² cfu/g of frozen concentrate or dried concentrate, and more preferably at least 10⁸, at least 10⁹, at least 10¹⁰, at least 10¹¹ or at least 10¹² cfu/g of frozen concentrate or dried concentrate.

### II. Methods of Producing Fermented Dairy Products for Storage at Ambient Temperature

Provided herein are methods of producing fermented dairy products for storage at ambient temperature. A fermented dairy product for storage at ambient temperature refers to a fermented dairy product that can be stored at ambient temperature without compromising the quality (e.g., healthy benefits, taste, texture) of the product. For example, the fermented dairy product for storage at ambient temperature may be stored at ambient temperature without or with minimal decreases in an amount of live bacteria in the product and/or without or with minimal decreases in the product's pH. In some embodiments, the fermented dairy product for storage at ambient temperature described herein may be stored at ambient temperature for a duration of time, such as for 6 months, a year, or more, without compromising the quality of the product. The methods provided herein include the use of lactose-deficient bacteria or cultures containing lactose-deficient bacteria, as described herein, to produce fermented dairy products for storage at ambient temperature.

### A. Initial Fermented Dairy Product

In aspects, the method of producing a fermented dairy product for storage at ambient temperature includes inoculating an initial fermented food product with a lactose-deficient bacterial strain or a composition containing a lactose-deficient bacterial strain, e.g., as described in Sections IA-1B. In some embodiments, the initial fermented dairy product is a food product before addition of one or more lactose-deficient bacterial strains as described in Section I-A or a culture containing lactose-deficient bacterial strains as described in Section I-B, and therefore does not contain lactose-deficient bacteria as defined herein. Thus, the initial fermented dairy food product does not necessarily contain lactose-deficient bacteria as defined herein.

In some embodiments, the initial fermented dairy product is any fermented dairy product that is intended for human consumption. In some embodiments, the initial fermented dairy product must be suitable for being inoculated with one or more lactose-deficient bacterial strains as described in the Section I-A or a culture containing a lactose-deficient bacterial strain as described in the Section I-B.

In some embodiments, the initial fermented dairy food product is produced by fermenting a milk substrate. Fermentation is carried out through the action of a bacteria starter by conversion of carbohydrates into acid. A “bacteria starter” is defined as a composition that is or includes of one or more bacteria which are able to start and perform the fermentation of a substrate. In some embodiments, the initial fermented dairy food product is a lactic acid-fermented food product. Thus, in some embodiments, the fermentation is carried out through the action of lactic acid bacteria starter by conversion of carbohydrates into lactic acid. The expression “lactic acid bacteria” (LAB) as used herein refers to food-grade bacteria producing lactic acid as the major metabolic end-product of carbohydrate fermentation. Lactic acid bacteria are well known in the art, and include strains of the Lactococcus genus, of the Streptococcus genus, of the Lactobacillus genus, of the Bifidobacterium genus, of the Leuconostoc genus, of the Enterococcus genus, of the Pediococcus genus, of the Brevibacterium genus and of the Propionibacterium genus.

In some embodiments, the initial fermented dairy food product is a dairy milk-based product. By “dairy milk-based product”, it is meant that the main component of the initial fermented dairy product is a dairy milk.

In some embodiments, the initial fermented dairy product is a fermented milk beverage, a yogurt, a cheese, sour cream, buttermilk, or fermented whey. Fermented dairy products are well known in the art and are manufactured through the action of a starter culture as defined herein on a milk substrate. It is contemplated herein that a milk substrate for producing the initial fermented dairy product may be any raw and/or processed milk material that can be subjected to fermentation, e.g., as described herein. Thus, useful milk substrates include, but are not limited to, solutions/suspensions of any milk products comprising protein, such as full fat or reduced fat milk, skim milk, buttermilk, reconstituted milk powder, condensed milk, dried milk, whey, whey permeate, whey protein concentrate, or cream. In some embodiments, the milk substrate may originate from any mammal, e.g., being substantially pure mammalian milk, or reconstituted milk powder.

The milk may be in the native state, reconstituted milk, or a skimmed milk. In some embodiments, the milk is supplemented with compounds necessary for the growth of bacteria or for the subsequent processing of fermented milk, such as fat, proteins of a yeast extract, peptone and/or a surfactant, for example.

In some embodiments, milk is a lacteal secretion obtained by milking any mammal, such as cows, sheep, goats, buffaloes, zebras, horses, donkeys, camels, and the like. In some embodiments, the milk substrate is a dairy milk. In some embodiments, the milk is cow milk.

In some embodiments, the milk substrate is a full fat milk. In some embodiments, the full fat milk is, is about, or is above about 3.5% fat by weight. In some embodiments, the full fat milk is or is about 3.25% fat by weight. In some embodiments, the milk substrate is a diluted milk.

For example, in some cases, a diluted milk may result in a milk having reduced fat or protein by weight compared to full fat milk. In some embodiments, the milk substrate is a reduced fat milk. For example, in some cases, the reduced fat milk is milk that has had a portion of its fat, e.g., milk fat, content removed. In some embodiments, the portion removed is, is about, or is at least 1%, 5%, 10%, 20%, 30%, 40%, 50%, 60%, 70%, 80%, or 90% of the fat content, e.g., by weight. In some embodiments, the reduced fat milk is or is about 1.6% fat by weight. In some embodiments, the reduced fat milk is or is about 1% fat by weight. In some embodiments, a reduced fat milk may be referred to as a low fat milk. For example, a reduced fat milk that is or is about 1% fat by weight may be referred to as a low fat milk. In some embodiments, the milk substrate is non-fat milk, also referred to as skim milk. For example, in some cases, the non-fat milk is milk that has had about 100% of its fat, e.g., milk fat, content removed. In some embodiments, the non-fat milk is, is about, or is less than 0.3% fat by weight. In some embodiments, the non-fat milk is or is about 0% fat by weight. In some embodiments, the percentage of fat by weight in a non-fat milk is negligible, e.g., at or below a level of detection or quantification. Suitable methods for measuring fat in milk, e.g., milk fat, include, but are not limited to, the Röse-Gottlieb reference method, the Gerber butyrometric method, the Mojonnier method, spectrophotometry, e.g., UV, and spectroscopy, e.g., infrared. In some embodiment, the full fat, reduced fat, low fat, and/or non-fat milk referred to herein is a dairy milk, optionally cow milk.

In some cases, the milk substrate may be homogenized and/or treated with heat, i.e., pasteurized. In some cases, homogenization is or includes intensive mixing to obtain a soluble suspension or emulsion. If homogenization is performed prior to fermentation, it may be performed to break up fat in the milk substrate, e.g., milk fat, into smaller sizes so that it no longer separates from the milk. This may be accomplished by forcing the milk at high pressure through small orifices.

In some embodiments, pasteurizing includes treatment of the milk substrate to reduce or eliminate the presence of live organisms, such as microorganisms. In some embodiments, pasteurization is attained by maintaining a specified temperature for a specified period of time. In some embodiments, the specific temperature is attained by heating. The temperature and duration may be selected in order to kill or inactivate certain bacteria, such as harmful bacteria. In some embodiments, the milk substrate is heated to a temperature between or between about 80 to about 120° C., inclusive. In some embodiments, the milk substrate is heated to a temperature between or between about 85 to about 100° C., inclusive. In some embodiments, the milk substrate is heated to a temperature between or between about 85 to about 95° C., inclusive. In some embodiments, the milk substrate is heated to a temperature of or of about 90° C., e.g., 90° C. +/−0.2° C. In some embodiments, the milk substrate is heated for or for about 5 to 20 minutes. In some embodiments, the milk substrate is heated for or for about 5 to 15 minutes. In some embodiments, the milk substrate is heated for or for about 10 minutes, e.g., 10 min ±1 min.

In some embodiments, the milk substrate may undergo thermization. In some cases, thermization is useful for preserving the properties of the milk substrate while significantly reducing, eliminating, or inactivating unwanted or harmful bacteria. In some embodiments, thermisation includes heating the milk substrate to a temperature of or of about 57° C. In some embodiments, thermization includes heating the milk substrate to a temperature in the range of or of about 64 to 69° C. In some embodiments, the milk substrate is heated for at least 15 seconds. For example, when the milk substrate is heated to a temperature of or of about 57° C., the duration of heating is for at least 15 seconds. In some embodiments, the milk substrate is heated for or for about 5 to 40 seconds. For example, when the milk substrate is heated to a temperature of or of about 64 to 69° C., the duration of heating is or is about 5 to 40 seconds. Thermization can be employed depending on milk quality and fermentation technology to be used.

In some embodiments, the milk substrate is commercial UHT milk. In some embodiments, the milk substrate is a fresh milk. In some embodiments, the milk substrate is a fresh commercial milk. In some embodiments, the milk substrate has a protein content of or of about 3.2% (per weight). In some embodiments, the milk substrate has a fat in content of or of about 3.8% (per weight). In some embodiments, the milk substrate has a lactose content of or of about 4.7% (per weight). In some embodiments, the milk substrate, such as a fresh milk, has undergone thermization. In some embodiments, the milk substrate, such as a fresh milk, has undergone pasteurization. In some embodiments, the milk substrate, such as a fresh milk, has undergone thermization and pasteurization.

In some embodiments, e.g., after heat treating (e.g., pasteurization, thermization), the milk substrate is cooled. In some embodiments, the milk substrate is cooled to a temperature between or between about 30 to about 50° C., inclusive. In some embodiments, the milk substrate is cooled to a temperature between or between about 35 to about 45° C., inclusive. In some embodiments, the milk substrate is cooled to a temperature of or of about 43° C. In some embodiments, the cooling is rapid cooling. In some embodiments, the milk substrate is cooled for less than 20, 15, 10, 9, 8, 7, 6, 5, 4, 3, 2, or 1 minute. In some embodiments, the milk substrate is cooled for less than 10 minutes. In some embodiments, the milk substrate is cooled for less than 5 minutes. In some embodiments, the milk substrate is cooled for or for about 10 minutes. In some embodiments, the milk substrate is cooled for or for about 5 minutes.

In some embodiments, the initial fermented dairy product is produced by fermentation of a milk substrate with a starter culture. In some embodiments, the starter culture includes lactic acid bacteria. In some embodiments, the starter culture includes one or more of a Streptococcus thermophilus strain, a strain from the Lactobacillus genus, a strain of Lactococcus lactis (e.g., a Lactococcus lactis subsp lactis strain, a Lactococcus lactis subsp hordniae strain), and a strain of Lactococcus cremoris (e.g., a Lactococcus cremoris subsp cremoris strain). In a particular embodiment, said fermented milk is obtained by fermentation of milk with a lactic acid bacteria starter selected from the group consisting of a starter culture comprising or consisting of Streptococcus thermophilus and Lactobacillus delbrueckii subsp bulgaricus, a starter culture comprising or consisting of Streptococcus thermophilus and Lactobacillus johnsonii, and a starter culture comprising or consisting of Streptococcus thermophilus and Limosilactobacillus fermentum.

As described herein, it was found that inoculating fermented dairy products, e.g., initial fermented dairy products, having low levels of galactose with live lactic acid bacteria incapable of fermenting lactose produced a fermented dairy product that could be stored at ambient temperature, e.g., room temperature, and maintain levels of viable bacteria and PH levels similar to those observed at the time of inoculation (see, e.g., Examples). Thus, in some embodiments, the Streptococcus thermophilus of a starter culture described herein is a galactose-metabolizing Streptococcus thermophilus strain. In some embodiments, the initial fermented dairy product is fermented with a starter culture including a galactose-metabolizing Streptococcus thermophilus. In some embodiments, the initial fermented dairy product is fermented with a starter culture including a galactose-metabolizing Streptococcus thermophilus and Lactobacillus delbrueckii subsp bulgaricus

As referred to herein, a “galactose-metabolizing Streptococcus thermophilus strain” is a Streptococcus thermophilus strain able to metabolize galactose. A galactose-metabolizing Streptococcus thermophilus strain may be any Streptococcus thermophilus strain having any or all of the features described herein. Examples of galactose-metabolizing Streptococcus thermophilus strains contemplated for use herein are described in published international application WO 2020/089279, which is incorporated herein by reference.

In some embodiments, the galactose-metabolizing Streptococcus thermophilus strain is a Streptococcus thermophilus strain able to grow on a galactose medium.

In some embodiments, the galactose-metabolizing Streptococcus thermophilus strain is defined by published international application WO 2019/122365.

In some embodiments, the galactose-metabolizing Streptococcus thermophilus strain is characterized by its ability to reach a pH of 5.2 in less than 5 hours, optionally with an average speed of acidification of at least 0.01 upH/min between pH 6.4 and 5.6, when inoculated at 1% (v/v) into a M17 oxoid medium supplemented with galactose 30 g/L and incubated at 43° C., in particular when tested by assay I. In some embodiments, the galactose-metabolizing Streptococcus thermophilus strain is characterized by its ability to reach a pH of 5.2 in less than 4 hours, optionally with an average speed of acidification of at least 0.01 upH/min between pH 6.4 and 5.6, when inoculated at 1% (v/v) into a M17 oxoid medium supplemented with galactose 30 g/L and incubated at 43° C., in particular when tested by assay I.

Assay I is as follows:
Streptococcus thermophilus strains characterized as galactose-positive (i.e., described as able to grow on a medium comprising galactose as the only source of carbohydrates) were grown overnight at 37° C. in M17 supplemented with sucrose 30 g/L;
the culture was washed (v/v) in tryptone-salt solution (tryptone 1 g/L, NaCl 8.5 g/L) as follows: the culture was centrifuged at 4000 rpm for 5 minutes; the pellet was resuspended in 10 ml of tryptone-salt solution;
the washed culture was inoculated at 1% (v/v) into 150 ml of M 17 oxoid supplemented with galactose 30 g/L;
the inoculated medium was incubated at 43° C. for 24 hours, and its pH monitored using a CINAC system (Alliance Instruments, France; pH electrode Mettler 405 DPAS SC, Toledo, Spain); the pH was measured and recorded every 5 minutes. Using the CINAC v2.07 software, the following parameters were specifically calculated: the time to reach a pH of 5.2 and the slope between pH 6.4 and pH 5.6 (UpH/minute) [Slope pH6.4-5.6].

In some embodiments, the galactose-metabolizing Streptococcus thermophilus has the ability to excrete galactose but to consume the excreted galactose to completion at most 9 hours after being inoculated at 1% (v/v) into a M17 medium supplemented with 0.5% (wt/wol) of lactose and incubated at 42° C., in particular when tested by assay II. In some embodiments, the galactose-metabolizing Streptococcus thermophilus strain is characterized by its ability to excrete galactose but to consume the excreted galactose to completion at most 8 hours after being inoculated at 1% (v/v) into a M17 medium supplemented with 0.5% (wt/wol) of lactose and incubated at 42° C., in particular when tested by assay II.

Assay II is as follows:
S. thermophilus strains characterized as galactose-positive are grown 12 hours at 42° C. in M17 supplemented with 0.5% (wt/vol) of lactose [1% (v/v) inoculation]; this step is repeated a second time in the same conditions;
the culture is inoculated at 1% (v/v) into a M17 medium supplemented with 0.5% (wt/vol) of lactose, and the inoculated medium is incubated at 42° C. up to 10 hours;
during fermentation, samples are withdrawn every 30 minutes to determine the galactose concentration; samples are centrifuged at 14000×g for 5 minutes, filtered sterilized through Phenex nylon 0.45 pm-pore size×15 mm diameter filters (Phenomenex®) and stored at −20° C. until further analysis; 10 ml of each sample are injected on an Agilent® 1100 HPLC. The elution is done through isocratic mode with pure H2O at 0.6 ml/min. Sugars are separated in 40 minutes onto a Pb2+ ion exchange column (SP0810 Shodex™ 300 mm×8 mm×7 pm). The concentration of galactose (if any) is determined (g/L). Concentration of galactose below 0.05 g/L is considered not measurable.

In some embodiments, the galactose-metabolizing Streptococcus thermophilus strain is characterized by its ability to reach a pH of 5.2 in less than 5 hours, in particular less than 4hours, optionally with an average speed of acidification of at least 0.01 upH/min between pH 6.4 and 5.6, when inoculated at 1% (v/v) into a M17 oxoid medium supplemented with galactose 30 g/L and incubated at 43° C., in particular when tested by assay I, and its ability not to excrete galactose, when inoculated at 1% (v/v) into a M17 medium supplemented with 0.5% (wt/wol) of lactose and incubated at 42° C., in particular when tested by assay II. In an embodiment, the galactose-metabolizing Streptococcus thermophilus strain is characterized by its ability to reach a pH of 5.2 in less than 5 hours, in particular less than 4 hours, optionally with an average speed of acidification of at least 0.01 upH/min between pH 6.4 and 5.6, when inoculated at 1% (v/v) into a M17 oxoid medium supplemented with galactose 30 g/L and incubated at 43° C., in particular when tested by assay Il and its ability to excrete galactose but to consume the excreted galactose to completion at most 9 hours, in particular at most 8 hours, after being inoculated at 1% (v/v) into a M17 medium supplemented with 0.5% (wt/wol) of lactose and incubated at 42° C., in particular when tested by assay II.

In some embodiments, the galactose-metabolizing Streptococcus thermophilus strain is the DSM32823 strain deposited at DSMZ on May 29, 2018 or mutants thereof. In some embodiments, the galactose-metabolizing Streptococcus thermophilus strain is the strain deposited under accession number DSM33851 at the DSMZ or mutants thereof. In some embodiments, the galactose-metabolizing Streptococcus thermophilus strain is the strain deposited under accession number DSM33852, at the DSMZ or mutants thereof. In some embodiments, the galactose-metabolizing Streptococcus thermophilus strain is the strain deposited under accession number DSM33853, at the DSMZ or mutants thereof. In some embodiments, the galactose-metabolizing Streptococcus thermophilus strain is the strain deposited under accession number DSM33854, at the DSMZ or mutants thereof. In some embodiments, a mutant is a strain derived from a deposited strain that has been manipulated, e.g., genotypically manipulated, but maintains the same or an improved phenotype, e.g., galactose-metabolizing, of the parent.

In some embodiments, the galactose content of the initial fermented dairy product is less than about 2%, e.g., less than 1.5%, 1%, or 0.5%. In some embodiments, the galactose content of the initial fermented dairy product is less than about 2%. In some embodiments, the galactose content of the initial fermented dairy product is less than about 1%. In some embodiments, the galactose content of the initial fermented dairy product is less than about 0.75%. In some embodiments, the galactose content of the initial fermented dairy product is less than about 0.5%. In some embodiments, the galactose content of the initial fermented dairy product is between 0 and 3%. In some embodiments, the galactose content of the initial fermented dairy product is between 0 and 2%. In some embodiments, the galactose content of the initial fermented dairy product is between 0 and 1%. In some embodiments, the galactose content of the initial fermented dairy product is between 0 and 0.75%. In some embodiments, the galactose content of the initial fermented dairy product is between 0 and 0.5%.

In some embodiments, the initial fermented dairy product contains one or more sugar substitutes.

In some embodiments, the pH of the initial fermented dairy product is between 3.5 and 5.0.

In some embodiments, the pH of the initial fermented dairy product is between 3.8 and 4.8.

In some embodiments, the pH of the initial fermented dairy product is between 4.0 and 4.8.

In some embodiments, the pH of the initial fermented food product is between 4.1 and 4.6. In some embodiments, the pH of the initial fermented food product is or is about 4.1, 4.2, 4.3, 4.4, 4.5, or 4.6. In some embodiments, the pH of the initial fermented food product is or is about 4.3, 4.4, 4.5, or 4.6. The pH can be determined using any pH meter.

In some embodiments, the initial fermented dairy product contains a level of bacteria that is or is less than about 1×10⁵ CFU per g of said initial fermented dairy product. In some embodiments, the initial fermented dairy product contains a level of bacteria that is or is less than about 1×10⁴ CFU per g of said initial fermented dairy product. In some embodiments, the initial fermented dairy product contains a level of bacteria that is or is less than about 1×10³ CFU per g of said initial fermented dairy product. By “level of bacteria” used herein, it is meant the total amount of bacteria as calculated as cfu/g of product. The cfu count can be measured by plating dilution(s) of the product to be tested on MRS/M17/PCA agar (Atlas, 2010 Handbook of Microbiological Media, Fourth Edition, pages 986, 1231 and 1402).

In some embodiments, the initial fermented dairy product naturally has a level of bacteria that is or is less than about 1×10⁴ CFU per g of food product.

In some embodiments, the initial fermented dairy product has a level of bacteria of more than 1×10⁵ CFU per g of food product. In some embodiments, the initial fermented dairy product has a level of bacteria of more than 1×10⁴ CFU per g of food product. In some embodiments, the initial fermented dairy product has a level of bacteria of more than 1×10³ CFU per g of food product. The presence of bacteria, in particular lactic acid bacteria, can result from the use of these microorganisms (in particular as starter) during the manufacture of the initial fermented dairy product, for example when the initial fermented dairy product results from fermentation of a substrate, e.g., as described above.

In some embodiments, the initial fermented dairy product is treated before inoculation with a lactose-deficient bacterial strain, e.g., lactose-deficient L. plantarum strain, as described in Section I-A or a culture containing a lactose-deficient bacterial stain, e.g., a lactose-deficient L. plantarum strain, as described in Section I-B to reduce the level of bacteria. In some embodiments, the level of bacteria is reduced to or to less than about 1×10⁵ CFU per g of said initial fermented dairy product. In some embodiments, the level of bacteria is reduced to or to less than about 1×10⁴ CFU per g of said initial fermented dairy product. In some embodiments, the level of bacteria is reduced to or to less than about 1×10³ CFU per g of said initial fermented dairy product.

By “treating”, it is meant any treatment which inactivates the bacteria contained in the initial fermented dairy product (e.g. which inhibits or reduces the bacteria growth or kills bacteria), so as to reduce the level of bacteria to no more than 1×10⁴ CFU per g of the initial fermented dairy product. Methods of treatment are well known in the art. In some embodiments, the initial fermented dairy product is treated using one or more methods selected from the group consisting of high-pressure sterilization, irradiation, ultra-filtration and heat-treating. In some embodiments, the initial fermented dairy product is heat-treated to reduce the level of bacteria to no more than 1×10⁴ CFU per g of the initial fermented dairy product.

By “heat-treating”, it is meant any treatment based on temperature which inactivates the bacteria contained in the initial fermented dairy product (e.g. which inhibits or reduces the bacteria growth or kills bacteria), so as to reduce the level of bacteria in the initial fermented dairy product to no more than 1×10⁴ CFU per g of the initial fermented dairy product.

### B. Adding/Inoculating Lactose-Deficient Bacteria to the Initial Fermented Dairy Product

In some embodiments, the initial fermented dairy product as defined herein is inoculated with a lactose-deficient bacterial strain, e.g., lactose-deficient L. plantarum strain, as described in Section I-A or a culture containing a lactose-deficient bacterial stain, e.g., a lactose-deficient L. plantarum strain, as described in Section I-B. In some embodiments, the initial fermented dairy product is inoculated with at least about 1.0×10⁵ CFU per g of a lactose-deficient bacterial strain, e.g., lactose-deficient L. plantarum strain, as described in Section I-A or a culture containing a lactose-deficient bacterial stain, e.g., a lactose-deficient L. plantarum strain, as described in Section I-B. In some embodiments, the initial fermented dairy product is inoculated with between about 1.0×10⁵ to about 1.0×10¹⁰ CFU per g, inclusive, of a lactose-deficient bacterial strain, e.g., lactose-deficient L. plantarum strain, as described in Section I-A or a culture containing a lactose-deficient bacterial stain, e.g., a lactose-deficient L. plantarum strain, as described in Section I-B. In some embodiments, the initial fermented dairy product is inoculated with between about 1.0×10⁵ to about 1.0×10⁹ CFU per g, inclusive, of a lactose-deficient bacterial strain, e.g., lactose-deficient L. plantarum strain, as described in Section I-A or a culture containing a lactose-deficient bacterial stain, e.g., a lactose-deficient L. plantarum strain, as described in Section I-B. In some embodiments, the initial fermented dairy product is inoculated with between about 1.0×10⁵ to about 1.0×10⁸ CFU per g, inclusive, of a lactose-deficient bacterial strain, e.g., lactose-deficient L. plantarum strain, as described in Section I-A or a culture containing a lactose-deficient bacterial stain, e.g., a lactose-deficient L. plantarum strain, as described in Section I-B. In some embodiments, the initial fermented dairy product is inoculated with between about 1.0×10⁵ to about 1.0×10⁷ CFU per g, inclusive, of a lactose-deficient bacterial strain, e.g., lactose-deficient L. plantarum strain, as described in Section I-A or a culture containing a lactose-deficient bacterial stain, e.g., a lactose-deficient L. plantarum strain, as described in Section I-B. In some embodiments, the initial fermented dairy product is inoculated with between about 1.0×10⁶ to about 1.0×10¹⁰ CFU per g, inclusive, of a lactose-deficient bacterial strain, e.g., lactose-deficient L. plantarum strain, as described in Section I-A or a culture containing a lactose-deficient bacterial stain, e.g., a lactose-deficient L. plantarum strain, as described in Section I-B. In some embodiments, the initial fermented dairy product is inoculated with or with about 1.0×10⁷ CFU per g of a lactose-deficient bacterial strain, e.g., lactose-deficient L. plantarum strain, as described in Section I-A or a culture containing a lactose-deficient bacterial stain, e.g., a lactose-deficient L. plantarum strain, as described in Section I-B. In some embodiments, when the initial fermented dairy product is inoculated with a culture containing a lactose-deficient bacterial stain, e.g., a lactose-deficient L. plantarum strain, as described in Section I-B, the inoculated amount in CFU per g refers to the lactose-deficient bacterial stain, e.g., a lactose-deficient L. plantarum strain, contained in the culture.

Within the context of the invention, “adding” is used interchangeably with “inoculating” (as well as “added” and “inoculated”) and means that the lactose-deficient bacterial strains or cultures containing a lactose-deficient bacterial strain as described herein are put in contact with the initial fermented dairy product. In some embodiments, one or more, e.g., 1, 2, 3, 4, 5, 6, 7, 8, 9 or 10, lactose-deficient bacterial strains, optionally contained in a culture, are inoculated into the initial fermented dairy product. In some embodiments, one lactose-deficient bacterial strain is added to the initial fermented dairy product. In some embodiments, two lactose-deficient bacterial strains are added to the initial fermented dairy product. In some embodiments, three lactose-deficient bacterial strains are added to the initial fermented dairy product. In some embodiments, four lactose-deficient bacterial strains are added to the initial fermented dairy product. In some embodiments, five lactose-deficient bacterial strains are added to the initial fermented dairy product. In some embodiments, the lactose-deficient bacterial strains are contained in a culture. In some embodiments, the lactose-deficient bacterial strain is a lactose-deficient L. plantarum as described herein.

When several (i.e., at least two) lactose-deficient bacterial strains are added to an initial fermented dairy product, the inoculated amount is the sum of each individual amount of inoculated lactose-deficient bacterial strains (as an example, addition of a lactose-deficient bacterial strain at 3×10⁵ cfu/g and of a second lactose-deficient bacterial strain at 7×10⁵ cfu/g leads to an inoculated amount of 1×10⁶ cfu/g). In an embodiment, the one or more lactose-deficient bacterial strains are added to the initial fermented dairy product in a total amount selected from the group consisting of at least 5×10⁵ CFU per g, at least 1×10⁶ CFU per g, at least 5×10⁶ CFU per g or at least 1×10⁷ CFU per g of the initial fermented dairy product. In an embodiment, the one or more lactose-deficient bacterial strains are added to the initial fermented dairy product in a total amount range selected from the group consisting of from 1×10⁵ to 1×10⁸ cfu per g, from 1×10⁶ to 1×10⁸ cfu per g and from 5×10⁶ to 1×10⁸ cfu per g of the initial fermented dairy product.

The one or more lactose-deficient bacterial strains as described herein can be inoculated into the initial fermented dairy product under any form, such as under frozen, dried, freeze-dried, liquid or solid format, in the form of pellets or frozen pellets, or in the form of a powder or dried powder. In some embodiments, the one or more lactose-deficient bacterial strains are added to the initial fermented dairy product, under liquid form, for example as bulk starter (i.e., a LAB culture previously propagated in a growth medium to obtain the required concentration of inoculation). In some embodiments, the one or more lactose-deficient bacterial strains are directly added to the initial fermented dairy product under the form of concentrates, for example frozen or dried concentrates. In some embodiments, the one or more lactose-deficient bacterial strains are added to the food product under liquid form as a dilution (e.g. in water or saline solution) of concentrates, such as of frozen or dried concentrates. The expression “directly inoculated” means that the one or more lactose-deficient bacterial strains are added into the initial fermented dairy product without previous propagation. The direct inoculation requires that the concentration of the one or more lactose-deficient bacterial strains be high enough. Thus, the concentration of lactose-deficient bacterial strains in the frozen or dried concentrate is in the range of 10⁸ to 10¹² cfu per g of concentrate, and more preferably at least 10⁸, at least 10⁹, at least 10¹⁰, at least 10¹¹ or at least 10¹² cfu/g of concentrate.

In some embodiments, said one or more lactose-deficient bacterial strains are aseptically added to the initial fermented dairy product. By “aseptically”, it is meant that no other micro-organisms than the one or more lactose-deficient bacterial strains are added to the food product, e.g. by using Tetra FlexDos™ aseptic in-line inoculation system.

### C. A Fermented Dairy Product for Storage at Ambient Temperature

The methods provided herein produce a fermented dairy product that can be stored at ambient temperature without impacting the quality of the product. A fermented dairy product for storage at ambient temperature refers to a fermented dairy product containing one or more lactose-deficient bacterial strains as defined herein, and for which the amount of lactose-deficient bacterial strains and/or the pH is not or is minimally changed when stored at ambient temperature.

In some embodiments, the fermented dairy product for storage at ambient temperature produced according to the methods provided herein has a decrease in the amount of inoculated lactose-deficient bacteria of less than or less than about 1.5, 1, 0.75, 0.5, 0.25 log compared to the level of inoculation when stored at ambient temperature. In some embodiments, the fermented dairy product for storage at ambient temperature produced according to the methods provided herein has a decrease in the amount of inoculated lactose-deficient bacteria of less than or less than about 1 log compared to the level of inoculation when stored at ambient temperature. In some embodiments, the fermented dairy product for storage at ambient temperature produced according to the methods provided herein has a decrease in the amount of inoculated lactose-deficient bacteria of less than or less than about 0.75 log compared to the level of inoculation when stored at ambient temperature. In some embodiments, the fermented dairy product for storage at ambient temperature produced according to the methods provided herein has a decrease in the amount of inoculated lactose-deficient bacteria of less than or less than about 0.5 log compared to the level of inoculation when stored at ambient temperature. In some embodiments, the fermented dairy product for storage at ambient temperature produced according to the methods provided herein has a decrease in the amount of inoculated lactose-deficient bacteria of less than or less than about 0.25 log compared to the level of inoculation when stored at ambient temperature. In some embodiments, the fermented dairy product for storage at ambient temperature produced according to the methods provided herein has a decrease in the amount of inoculated lactose-deficient bacteria in the range of about 0 to 1 log compared to the level of inoculation when stored at ambient temperature. In some embodiments, the fermented dairy product for storage at ambient temperature produced according to the methods provided herein has a decrease in the amount of inoculated lactose-deficient bacteria in the range of about 0.25 to 1 log compared to the level of inoculation when stored at ambient temperature. In some embodiments, the fermented dairy product for storage at ambient temperature produced according to the methods provided herein has a decrease in the amount of inoculated lactose-deficient bacteria in the range of about 0.5 to 1 log compared to the level of inoculation when stored at ambient temperature. In some embodiments, the fermented dairy product for storage at ambient temperature produced according to the methods provided herein has a decrease in the amount of inoculated lactose-deficient bacteria in the range of about 0.75 to 1 log compared to the level of inoculation when stored at ambient temperature.

In some embodiments, the fermented dairy product for storage at ambient temperature produced according to the methods provided herein changes by less than or less than about 0.2, 0.15, 0.1, 0.05 UpH compared to the pH at the time of inoculation with a lactose-deficient bacterial strain when stored at ambient temperature. In some embodiments, the fermented dairy product for storage at ambient temperature produced according to the methods provided herein changes by less than or less than about 0.2 UpH compared to the pH at the time of inoculation with a lactose-deficient bacterial strain when stored at ambient temperature. In some embodiments, the fermented dairy product for storage at ambient temperature produced according to the methods provided herein changes by less than or less than about 0.15 UpH compared to the pH at the time of inoculation with a lactose-deficient bacterial strain when stored at ambient temperature. In some embodiments, the fermented dairy product for storage at ambient temperature produced according to the methods provided herein changes by less than or less than about 0.1 UpH compared to the pH at the time of inoculation with a lactose-deficient bacterial strain when stored at ambient temperature. In some embodiments, the fermented dairy product for storage at ambient temperature produced according to the methods provided herein changes by less than or less than about 0.05 UpH compared to the pH at the time of inoculation with a lactose-deficient bacterial strain when stored at ambient temperature. In some embodiments, the fermented dairy product for storage at ambient temperature produced according to the methods provided herein changes in the range of about 0 to about 0.2 UpH compared to the pH at the time of inoculation with a lactose-deficient bacterial strain when stored at ambient temperature. In some embodiments, the fermented dairy product for storage at ambient temperature produced according to the methods provided herein changes in the range of about 0 to about 0.15 UpH compared to the pH at the time of inoculation with a lactose-deficient bacterial strain when stored at ambient temperature. In some embodiments, the fermented dairy product for storage at ambient temperature produced according to the methods provided herein changes in the range of about 0 to about 0.1 UpH compared to the pH at the time of inoculation with a lactose-deficient bacterial strain when stored at ambient temperature. In some embodiments, the fermented dairy product for storage at ambient temperature produced according to the methods provided herein changes in the range of about 0 to about 0.05 UpH compared to the pH at the time of inoculation with a lactose-deficient bacterial strain when stored at ambient temperature. In some embodiments, the fermented dairy product for storage at ambient temperature produced according to the methods provided herein changes by about 0.17, 0.16, 0.15, 0.14, 0.13, 0.12, 0.11, or 0.1 UpH compared to the pH at the time of inoculation with a lactose-deficient bacterial strain when stored at ambient temperature. In some embodiments, the fermented dairy product for storage at ambient temperature produced according to the methods provided herein changes by about 0.17, 0.16, or 0.15 UpH compared to the pH at the time of inoculation with a lactose-deficient bacterial strain when stored at ambient temperature. In some embodiments, the fermented dairy product for storage at ambient temperature produced according to the methods provided herein changes by about 0.16 UpH compared to the pH at the time of inoculation with a lactose-deficient bacterial strain when stored at ambient temperature.

In some embodiments, ambient temperature is room temperature. In some embodiments, the ambient temperature is 25° C.

In some embodiments, the storage at ambient temperature is for a duration of 1, 2, 3, 4, 5, 6, 7, 8, 9, 10, or 11 months or more. In some embodiments, the storage at ambient temperature is for a duration of a year or more, e.g., 1.5 or 2 years. In some embodiments, the storage at ambient temperature is for a duration of ranging from about 1 month to about 1 year. In some embodiments, the storage at ambient temperature is for a duration of ranging from about 2 months to about 1 year. In some embodiments, the storage at ambient temperature is for a duration of ranging from about 3 months to about 1 year. In some embodiments, the storage at ambient temperature is for a duration of ranging from about 4 months to about 1 year. In some embodiments, the storage at ambient temperature is for a duration of ranging from about 5 months to about 1 year. In some embodiments, the storage at ambient temperature is for a duration of ranging from about 6 months to about 1 year. In some embodiments, the storage at ambient temperature for a duration of ranging from about 1 month to 6 months. In some embodiments, the storage at ambient temperature is for a duration of ranging from about 1 month to 5 months. In some embodiments, the storage at ambient temperature is for at least or about 5 months, e.g., 150 days. In some embodiments, the duration of storage is calculated starting at the day of inoculation with the lactose-deficient bacterial strain or culture, e.g., inoculation day is day 0.

In some embodiments, the fermented dairy product for storage at ambient temperature produced according to the methods provided herein has a decrease in the amount of inoculated lactose-deficient bacteria and/or a change in pH as described herein. In some embodiments, the fermented dairy product for storage at ambient temperature produced according to the methods provided herein has a decrease in the amount of inoculated lactose-deficient bacteria and a change in pH as described herein. In some embodiments, the fermented dairy product for storage at ambient temperature produced according to the methods provided herein has a decrease in the amount of inoculated lactose-deficient bacteria of less than or less than about 1.5, 1, 0.75, 0.5, 0.25 log compared to the level of inoculation and/or changes by less than or less than about 0.2, 0.15, 0.1, 0.05 UpH compared to the pH at the time of inoculation with a lactose-deficient bacterial strain when stored at 25° C. for at least or about 150 days. In some embodiments, the fermented dairy product for storage at ambient temperature produced according to the methods provided herein has a decrease in the amount of inoculated lactose-deficient bacteria of less than or less than about 1.5, 1, 0.75,0.5, 0.25 log compared to the level of inoculation and changes by less than or less than about 0.2, 0.15, 0.1, 0.05 UpH compared to the pH at the time of inoculation with a lactose-deficient bacterial strain when stored at 25° C. for at least or about 150 days.

In some embodiments, the food product is stored under a sealed format (i.e., in closed sterile container).

In some embodiments, the pH of the fermented dairy product stored at ambient temperature is determined by pH meter (Mettler Toledo, SevenEasy) and compared to the pH of the fermented dairy product at day 0, day of inoculation.

In some embodiments, the level of lactose-deficient bacteria in the fermented dairy product stored at ambient temperature is determined by CFU count, for examples as in Example 1 or measured by plating dilution(s) of the product to be tested on MRS/M17/PCA agar (Atlas, 2010 Handbook of Microbiological Media, Fourth Edition, pages 986, 1231 and 1402), and compared with the level of lactose-deficient bacteria added at day 0, day of inoculation. In some embodiments, the level of lactose-deficient bacteria in the fermented dairy product stored at ambient temperature is at least 1×10⁴ CFU/g. In some embodiments, the level of lactose-deficient bacteria in the fermented dairy product stored at ambient temperature is between about 1×10⁴ CFU/g to about 1×10¹⁰ CFU/g. In some embodiments, the level of lactose-deficient bacteria in the fermented dairy product stored at ambient temperature is not decreased by more than 1.5 log and is at least 1×10⁴ CFU/g. the level of lactose-deficient bacteria in the fermented dairy product stored at ambient temperature is not decreased by more than 1 log and is at least 1×10⁴ CFU/g. In some embodiments, the level of lactose-deficient bacteria in the fermented dairy product stored at ambient temperature is not decreased by more than 1.5 log and is between about 1×10⁴ CFU/g to about 1×10¹⁰ CFU/g, inclusive. the level of lactose-deficient bacteria in the fermented dairy product stored at ambient temperature is not decreased by more than 1 log and is between about 1×10⁴ CFU/g to about 1×10¹⁰ CFU/g, inclusive.

The invention also relates to a fermented dairy product, as defined herein or as obtained by the methods of the invention and containing one or more lactose-deficient bacterial strains as defined herein, e.g., a lactose-deficient L. plantarum strain.

In some embodiments, the fermented dairy product for storage at ambient temperature, as defined herein or as obtained by a method of the invention, contains a lactose-deficient L. plantarum strain. In some embodiments, the lactose-deficient L. plantarum strain is the strain DSM33967 deposited at the DSMZ on on Aug. 11, 2021, or a mutant thereof as defined herein of the DSM33967 strain.

In some embodiments, the fermented dairy product is a fermented milk beverage, a yogurt, a cheese, sour cream, buttermilk, or a fermented whey. In some embodiments, the fermented dairy product is a fermented milk beverage. In some embodiments, a fermented dairy product is a yogurt.

### III. Kits

Also provided are kits including the bacteria and cultures described herein, for example at Sections I-A and I-B and Section II-A, which may further include instructions on methods of using the bacteria, such as uses described herein. The kits described herein may also include other materials desirable from a commercial and user standpoint, including other buffers, diluents, filters, needles, syringes, and package inserts with instructions for performing any methods described herein.

### Deposit and Expert Solution

The following deposits were made according to the Budapest treaty on the international recognition of the deposit of microorganisms for the purposes of patent procedure.
Lactiplantibacillus plantarum strain DGCC13484 deposited by DuPont Nutrition Biosciences ApS under accession number DSM33967 on Aug. 11, 2021, at the DSMZ [Deutsche Sammlung von Mikroorganismen und Zellkulturen GmbH, Inhoffenstrasse 7B, D-38124 Braunschweig-Germany]; and
Streptococcus thermophilus strain deposited by DuPont Nutrition Biosciences ApS under accession number DSM32823 on May 29, 2018, at the DSMZ.

It is requested that the biological material shall be made available only by the issue of a sample to an expert nominated by the requester. In respect to those designations in which a European Patent is sought, a sample of the deposited microorganism will be made available until the publication of the mention of the grant of the European patent or until the date on which application has been refused or withdrawn or is deemed to be withdrawn, only by the issue of such a sample to an expert nominated by the person requesting the sample, and approved either i) by the Applicant and/or ii) by the European Patent Office, whichever applies (Rule 32 EPC).

### EXAMPLES

The following examples are included for illustrative purposes only and are not intended to limit the scope of the invention.

### Test A (Assay for Lactose Metabolism)

Cells of a fresh overnight culture of the Lactiplantibacillus plantarum strain are harvested by centrifugation at 6000 g for 10 min. Then, resuspended in same volume of phosphate buffer (3M™ Flip-Top Phosphate Buffer) to remove medium. Inoculate 2% v/v of the resuspend cells into MRS contains 1% lactose, and incubated at 37° C. for 48 h. The wild type L. plantarum strain (DSM 33121) was used as lactose positive reference. Optical density (O.D) was measured by a spectrophotometer at 595 nm. The growth of L. plantarum strain (ΔO.D48h) was calculated by O.D48h-O.D0h. The relative lactose metabolism activity of lactose deficient strain was calculated by ΔO.D48h of lactose deficient strain/ΔO.D48h of reference.

### Example 1: Production of a Fermented Dairy Product for Storage at Ambient Temperature

The ability to produce a yogurt having a stable pH and concentration of beneficial microorganisms during long-term storage at ambient temperature was assessed.

### A. Materials and Methods

Yogurts were prepared by fermenting commercial UHT milk (protein 3.2%, fat 3.8%, lactose 4.7%) inoculated with 2 U/100 L of an exemplary industrial freeze-dried galactose-metabolizing Streptococcus thermophilus strain (DSM32823). Fermentation was terminated once a pH between 4.3 and 4.5 was reached. The yogurt was heat treated at 90° C. for 10 minutes and then cooled to room temperature. After heat treating, the yogurt was inoculated at 1×10⁷ CFU/ml with an exemplary lactose-deficient L. plantarum strain (DSM33967) or an exemplary L. rhamnosus strain (ATCC53103). The sugar content of the produced experimental yogurt was determined and is described in Table E1 below.

For further comparison, a commercial ambient yogurt having 2.8% protein, 3% fat and a pH 4.3, which was not fermented with a galactose-metabolizing Streptococcus thermophilus strain, was inoculated at 1×10⁷ CFU/ml with exemplary L. plantarum strain (DSM33967). The sugar content of the yogurt was determined and is described in Table E1.

### Table E1: Sugar Content of Ambient Yogurts Tested

| | Lactose | Glucose | Galactose | Sucrose | Fructose |
|---|---|---|---|---|---|
| Commercial | 4.22 | 0.33 | 0.44 | 7.78 | — |
| ambient | | | | | |
| yogurt | | | | | |
| Experimental | 3.85 | — | 0.26 | — | — |
| Ambient yogurt | | | | | |

The inoculated yogurts were mixed, sealed, and stored at 25° C. for at least 150 days. These conditions represent average ambient storage conditions when food products are stored out of the fridge or out of cold rooms.

The pH and the amount of L. plantarum and L. rhamnosus were determined after 60, 90,120, and 150 days in storage. pH was determined by pH meter (Mettler Toledo, SevenEasy). Bacterial concentrations (log CFU) were determine by serially diluting a yogurt sample with sterile saline (101 to 107 dilution), inoculating petri dishes containing MRS agar (1.5%) with 1 mL of the diluted sample, and incubating the plates at 37° C. anaerobically for 2 days before cell counting.

Experiments were performed in triplicate.

### B. Results

### Ambient Yogurt

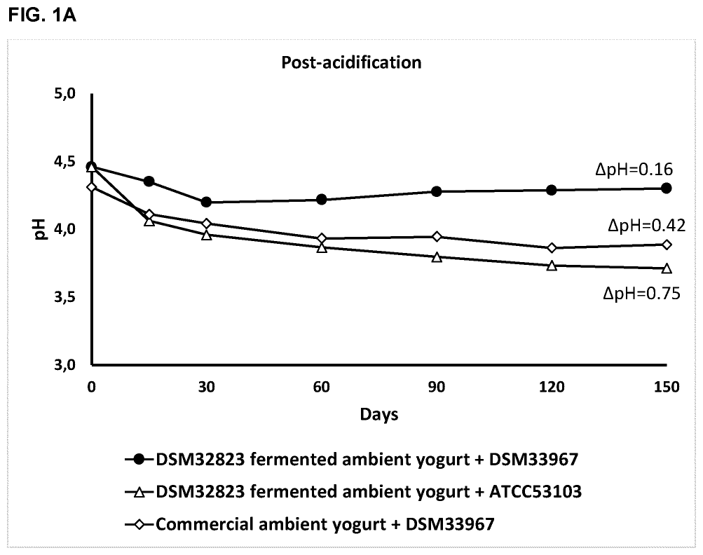

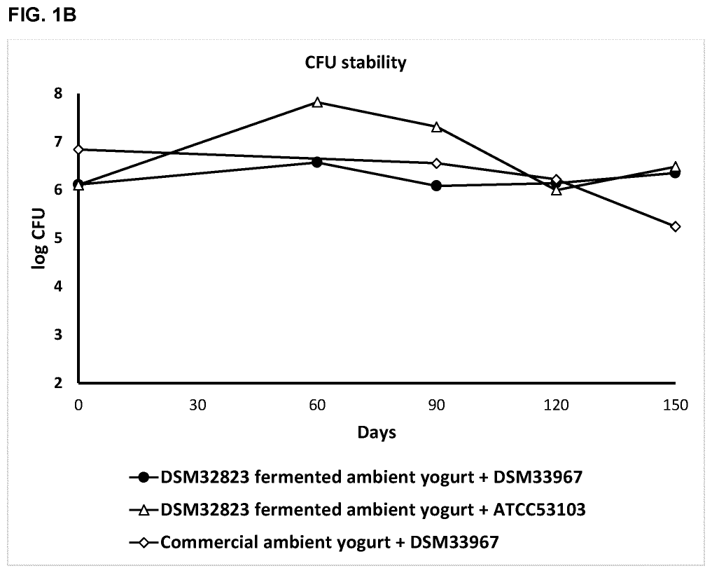

The pH and viable bacterial concentration (log CFU) determined over the duration of storage at ambient temperature for each yogurt tested are shown in FIGS. 1A and 1B, respectively.

As can be seen in FIG. 1A, the total change in pH in yogurts prepared with the exemplary galactose-metabolizing Streptococcus thermophilus and exemplary L. plantarum strains (ΔpH=0.16) was at least two-fold less compared to the changes in pH observed in yoghurts prepared with the exemplary galactose-metabolizing Streptococcus thermophilus and exemplary L. rhamnosus strain (ΔpH=0.42) or the commercial yoghurt inoculated with the exemplary L. plantarum (ΔpH=0.75).

As shown in FIG. 1B, the viable concentration of added bacteria (L. plantarum or L. rhamnosus) was more stable over time in yoghurts prepared with the exemplary galactose-metabolizing Streptococcus thermophilus and exemplary L. plantarum strains than yoghurts prepared with the exemplary galactose-metabolizing Streptococcus thermophilus and exemplary L. rhamnosus strain or the commercial inoculated with the exemplary lactose-deficient L. plantarum.

These results are supportive of the use of lactose-deficient L. plantarum strains in combination with fermented dairy products with decreased levels of residual sugars to produce fermented food products having stable pH and microorganism concentration during long-term storage at ambient temperature.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. Although the invention may be described in connection with specific preferred embodiments, it should be understood that the invention as claimed should not be un-duly limited to such specific embodiments. Indeed, various modifications of the described modes for carrying out the invention which are obvious to those skilled in molecular biology or related fields are intended to be within the scope of the following claims.

### NUMBERED EMBODIMENTS OF THE INVENTION

1. A method of producing a fermented dairy product for storage at ambient temperature, comprising inoculating an initial fermented dairy product with one or more lactose-deficient bacterial strains, wherein the initial fermented food product comprises between about 0% and about 1% galactose.

2. The method of embodiment 1, wherein the one or more lactose-deficient bacterial strains have a reduced or complete inability to metabolize lactose.

3. The method of embodiment 1 or embodiment 2, wherein the one or more lactose-deficient bacterial strains have a lactose metabolic activity of about or less than about 50%, 40%, 30%, 20%, or 10%, or no lactose metabolic activity compared to a lactose metabolic activity of a lactose-metabolizing bacteria comprising a beta-galactosidase protein having the amino acid sequence encoded by the sequence of nucleotides set forth by SEQ ID NO:1.

4. The method of any one of embodiments 1-3, wherein the one or more lactose-deficient bacterial strains comprise a beta-galactosidase protein having an amino acid sequence with at least about 70%, such as 75%, 80%, 85%, 90%, 95%, 96, 97%, 98%, 99%, 99.5%, or 100% sequence identity to the amino acid sequence encoded by the sequence of nucleotides set forth by SEQ ID NO:1.

5. The method of any one of embodiments 1-4, wherein the one or more lactose-deficient bacterial strains comprise a beta-galactosidase protein that has one or more amino acid substitution, addition or deletion compared to the amino acid sequence encoded by the sequence of nucleotides set forth by SEQ ID NO:7 and 8, or 1.

6. The method of any one of embodiments 1-5, wherein the one or more lactose-deficient bacterial strains comprise one or more mutation in the sequence of nucleotides of the promoter region of the beta-galactosidase gene lacL/M (PlacLM), which promoter region is defined by any one of SEQ ID NO:10 or SEQ ID NO:11.

7. The method of embodiment 6, wherein the mutation is in the-10 region of the promoter, such as a two-nucleotide mutation GG to AA at position 272-273 of SEQ ID NO:11.

8. The method of any one of embodiments 1-7, wherein the one or more lactose-deficient bacterial strains comprise a sequence of nucleotides of the promoter region of the beta-galactosidase gene lacL/M (PlacLM) defined by SEQ ID NO:10, or a sequence of nucleotides with at least about 70%, such as 75%, 80%, 85%, 90%, 95%, 96, 97%, 98%, 99%, 99.5%, or 100% sequence identity to the sequence of nucleotides defined by SEQ ID NO:10, or a sequence of nucleotides with at least about 70%, such as 75%, 80%, 85%, 90%, 95%, 96,97%, 98%, 99%, 99.5%, or 100% sequence identity to the sequence of nucleotides defined by position 261-281 or of position 269-279 of SEQ ID NO:10.

9. The method of any one of embodiments 1-8, wherein the one or more lactose-deficient bacterial strains comprise or consist of a lactose-deficient L. plantarum strain.

10. The method of embodiment 9, wherein the lactose-deficient L. plantarum strain is the strain deposited at the DMSZ under accession number DSM33967 or a mutant thereof.

11. The method of any one of embodiments 1-10, wherein the lactose-deficient bacterial strain is inoculated in an amount between about 1×10⁵ to about 1×10¹⁰ CFU/g of the initial fermented dairy product, optionally at an amount of about 1×10⁷ CFU/g of the initial fermented dairy product.

12. The method of any one of embodiments 1-11, wherein the lactose-deficient bacterial strain is inoculated aseptically to the initial fermented food product.

13. The method of any one of embodiments 1-12, wherein the initial fermented food product comprises a level of bacteria of at most 1×10⁴ CFU/g.

14. The method of any one of embodiments 1-13, wherein the initial fermented dairy product is produced by fermenting a milk substrate with a starter culture comprising a galactose-metabolizing Streptococcus thermophilus strain.

15. The method of embodiment 14, wherein the galactose-metabolizing Streptococcus thermophilus strain has an ability to consume at least 50% of galactose at the maximum speed of lactose consumption (VₘₐₓLacₕ) as determined by assay I and optionally an ability to consume at least 70% of galactose at the end of lactose consumption (V₀Lacₕ) determined by assay I.

16. The method of embodiment 14 or embodiment 15, wherein the galactose-metabolizing Streptococcus thermophilus strain is the strain deposited at the DMSZ under accession number DSM32823 or a mutant thereof.

17. The method of any of embodiments 1-16, wherein the one or more lactose-deficient bacterial strains inoculated into the fermented dairy product for storage at ambient temperature decreases by 1.5, 1, 0.75, 0.5, 0.25 log or less when stored at 25° C. for at least or about 150 days.

18. The method of any of embodiments 1-17, wherein a pH of the fermented dairy product for storage at ambient temperature changes by 0.2, 0.15, 0.1, 0.05 UpH or less when stored at 25° C. for at least or about 150 days.

19. The method of any one of embodiments 1-18, wherein the initial fermented dairy product is a yogurt or fermented milk beverage.

20. A culture or kit of parts comprising a lactose-deficient bacterial strain, wherein the lactose deficient bacterial strain has a lactose metabolic activity of about or less than about 50%, 40%, 30%, 20%, or 10% or no lactose metabolic activity compared to a lactose metabolic activity of a lactose-metabolizing bacteria comprising a beta-galactosidase protein having the amino acid sequence encoded by the sequence of nucleotides set forth by SEQ ID NO:1.

21. The culture or kit of parts of embodiment 20, wherein the lactose-deficient bacterial strain comprises a beta-galactosidase protein having an amino acid sequence with at least about 70%, such as 75%, 80%, 85%, 90%, 95%, 96, 97%, 98%, 99%, 99.5%, or 100% sequence identity to the amino acid sequence encoded by the sequence of nucleotides set forth by SEQ ID NO:1.

22. The culture or kit of parts of embodiment 20 or embodiment 21, wherein the one or more lactose-deficient bacterial strains comprise a beta-galactosidase protein that has one or more amino acid substitution, addition or deletion compared to the amino acid sequence encoded by the sequence of nucleotides set forth by SEQ ID NO:7 and 8, or 1.

23. The culture or kit of parts of embodiments 20-22, wherein the one or more lactose-deficient bacterial strains comprise one or more mutation in the sequence of nucleotides of the promoter region of the beta-galactosidase gene lacL/M (PlacLM), which promoter region is defined by any one of SEQ ID NO:10 or SEQ ID NO:11.

24. The culture or kit of parts of embodiment 23, wherein the mutation is in the −10 region of the promoter, such as a two-nucleotide mutation GG to AA at position 272-273 of SEQ ID NO:11.

25. The culture or kit of parts of embodiments 20-24, wherein the one or more lactose-deficient bacterial strains comprise a sequence of nucleotides of the promoter region of the beta-galactosidase gene lacL/M (PlacLM) defined by SEQ ID NO:10, or a sequence of nucleotides with at least about 70%, such as 75%, 80%, 85%, 90%, 95%, 96, 97%, 98%, 99%, 99.5%, or 100% sequence identity to the sequence of nucleotides defined by SEQ ID NO:10,or a sequence of nucleotides with at least about 70%, such as 75%, 80%, 85%, 90%, 95%, 96, 97%, 98%, 99%, 99.5%, or 100% sequence identity to the sequence of nucleotides defined by position 261-281 or of position 269-279 of SEQ ID NO:10.

26. The culture or kit of parts of any one of embodiments 20-25, wherein the lactose-deficient bacterial strain is a lactose-deficient L. plantarum strain.

27. The culture or kit of parts of embodiment 26, wherein the lactose-deficient L. plantarum strain is the strain deposited at the DMSZ under accession number DSM33967 or a mutant thereof.

28. The kit of parts of any one of embodiments 20-27, further comprising a galactose-metabolizing Streptococcus thermophilus strain.

29. The kit of parts of any one of embodiments 20-28, wherein the galactose-metabolizing Streptococcus thermophilus strain has an ability to consume at least 50% of galactose at the maximum speed of lactose consumption (VₘₐₓLacₕ) as determined by assay I and optionally an ability to consume at least 70% of galactose at the end of lactose consumption (V₀Lacₕ) determined by assay I.

30. The kit of parts of any one of embodiments 20-29, wherein the galactose-metabolizing Streptococcus thermophilus strain is the strain deposited at the DMSZ under accession number DSM32823 or a mutant thereof.

31. Use of a culture of any one of embodiments 20-30 or kit of parts of any one of embodiments 20-27 to produce a fermented dairy product for storage at ambient temperature.

32. A fermented dairy product for storage at ambient temperature obtained by the method of any one of embodiments 1-19 or the use of embodiment 31.

33. A fermented dairy product for storage at ambient temperature comprising a lactose-deficient bacterial strain, wherein the lactose deficient bacterial strain has a lactose metabolic activity of about or less than about 50%, 40%, 30%, 20%, or 10%, or no lactose metabolic activity compared to a lactose metabolic activity of a lactose-metabolizing bacteria comprising a beta-galactosidase protein having the amino acid sequence encoded by the sequence of nucleotides set forth by SEQ ID NO:1.

34. The fermented dairy product of embodiment 33, wherein the one or more lactose-deficient bacterial strains comprise a beta-galactosidase protein having an amino acid sequence with at least about 70%, such as 75%, 80%, 85%, 90%, 95%, 96, 97%, 98%, 99%, 99.5%, or 100% sequence identity to the amino acid sequence encoded by the sequence of nucleotides set forth by SEQ ID NO:1.

35. The fermented dairy product of embodiment 33 or embodiment 34, wherein the one or more lactose-deficient bacterial strains comprise a beta-galactosidase protein that has one or more amino acid substitution, addition or deletion compared to the amino acid sequence encoded by the sequence of nucleotides set forth by SEQ ID NO:7 and 8, or 1.

36. The fermented dairy product of embodiments 33-35, wherein the one or more lactose-deficient bacterial strains comprise one or more mutation in the sequence of nucleotides of the promoter region of the beta-galactosidase gene lacL/M (PlacLM), which promoter region is defined by any one of SEQ ID NO:10 or SEQ ID NO:11.

37. The fermented dairy product of embodiment 33-36, wherein the mutation is in the −10region of the promoter, such as a two-nucleotide mutation GG to AA at position 272-273 of SEQ ID NO:11.

38. The fermented dairy product of embodiments 33-37, wherein the one or more lactose-deficient bacterial strains comprise a sequence of nucleotides of the promoter region of the beta-galactosidase gene lacL/M (PlacLM) defined by SEQ ID NO:10, or a sequence of nucleotides with at least about 70%, such as 75%, 80%, 85%, 90%, 95%, 96, 97%, 98%, 99%, 99.5%, or 100% sequence identity to the sequence of nucleotides defined by SEQ ID NO:10, or a sequence of nucleotides with at least about 70%, such as 75%, 80%, 85%, 90%, 95%, 96, 97%, 98%, 99%, 99.5%, or 100% sequence identity to the sequence of nucleotides defined by position 261-281 or of position 269-279 of SEQ ID NO:10.

39. The fermented dairy product of any one of embodiments 36-38, wherein the lactose-deficient bacterial strain is a lactose-deficient L. plantarum strain.

40. The fermented dairy product of any one of embodiments 36-39, wherein the lactose-deficient L. plantarum strain is the strain deposited at the DMSZ under accession number DSM33967 or a mutant thereof.

41. The fermented dairy product of any one of embodiments 36-40, wherein the fermented dairy product for storage at ambient temperature is a yogurt or fermented milk beverage.

42. The fermented food product for storage at ambient temperature of any one of embodiments 36-41, comprising between about 0% and about 1% galactose.

43. The fermented dairy product for storage at ambient temperature of any one of embodiments 36-42, wherein the lactose-deficient L. plantarum strain in the fermented dairy product for storage at ambient temperature decreases by 1.5, 1, 0.75, 0.5, 0.25 log or less when stored at 25° C. for at least or about 150 days.

44. The fermented dairy product for storage at ambient temperature of any one of embodiments 36-43, wherein a pH of the fermented dairy product for storage at ambient temperature changes by 0.2, 0.15, 0.1, 0.05 or less UpH when stored at 25° C. for at least or about 150 days.

45. The fermented dairy product for storage at ambient temperature of any one of embodiments 36-44, wherein the fermented dairy product for storage at ambient temperature is a functional food.

46. The fermented dairy product for storage at ambient temperature of any one of embodiments 36-45, wherein the fermented dairy product for storage at ambient temperature comprises one or more sugar substitutes, optionally wherein the one or more sugar substitutes comprises erythritol.

47. A bacterial strain deposited at the DMSZ under accession number DSM33967 or a strain having all of the identifying characteristics of the L. plantarum strain deposited at DSMZ under accession number DSM33967.

48. A method for manufacturing a lactose-deficient bacterial strain comprising:
a) providing a lactic acid bacterial strain as the mother strain, such as Lactiplantibacillus plantarum DSM 33121;
b) introducing one or more mutations in the sequence of nucleotides of the promoter region of the beta-galactosidase gene lacL/M (PlacLM), such as the promoter region defined by any one of SEQ ID NO:10 or SEQ ID NO:11 of the strain; and
c) screening for a lactose-deficient bacterial strain have a reduced or complete inability to metabolize lactose.

49. The method of embodiment 48, wherein the mutation is in the-10 region of the promoter, such as a two-nucleotide mutation GG to AA at position 272-273 of SEQ ID NO:11.

50. The method of embodiment 48 or 49, wherein the lactose-deficient bacterial strain comprise a sequence of nucleotides of the promoter region of the beta-galactosidase gene lacL/M (PlacLM) defined by SEQ ID NO:10, or a sequence of nucleotides with at least about 70%, such as 75%, 80%, 85%, 90%, 95%, 96, 97%, 98%, 99%, 99.5%, or 100% sequence identity to the sequence of nucleotides defined by SEQ ID NO:10, or a sequence of nucleotides with at least about 70%, such as 75%, 80%, 85%, 90%, 95%, 96, 97%, 98%, 99%, 99.5%, or 100% sequence identity to the sequence of nucleotides defined by position 261-281 or of position 269-279 of SEQ ID NO:10.

SEQUENCES
> SEQ ID NO: 1 lac operon of Lactiplantibacillus plantarum
DSM33967
TCATTTGGTGCTTTCACGTTCTACCAACGTCGTTCCCAGTTCAATGCGCTGGGCTGCTTG
ACGATCATCTTGGAATCGGCTCACCAACAAATCGATGGCCGTCTTTCCCATCAGTTCAGT
CTCAACATTGACCGAGCTAATCTCTGGATACACATATTCGGCCAGCGTCGTGTTATTGAA
ACTAAACAACTTGACCCGCTGTGGCACAGCAATATCAGCTTGCTGTAACGCTTTGAGCGC
ACCGGCTGCCATCGGATCATTAGCGATAAAGAAAGCGTGTGGTAAGCGGTCACCTAACAT
TTCAATGGCCCGTTTCATCATCAGATAACCAGATTGACTAGTGTAATCACCAGCTAATAC
ATACTCAGCATGATAGGCATGATGCCGCTGCAACGCCTGTTCAAAGCTTTGCTGACGTTG
ATTGGGAATCTCCACCTGCTGATCAGTGGACCACTCTTTGCCATAAATCATACCGATATC
TCGGATACCACGCCGCCAAAAATAATCAACGACGCGGTTAGTAGCAAACGTAAAATCAGT
CACCACACTGCTGAATCCCTGAGCAAAGTGATCGTCATCAACAAAAACAAGCTGGCTTGA
CAAACGACTCAACTGTTGTACTTGGACAGGGCTAAATTTTCCCACCGCCACGATTGCATC
AATATCATCGCCAACCTGATCTAAATTATTCTGGAAGATACTAGTCGTCGTAAAACCACA
CACTTGAGCTTGATGCTCAATTCCTTTACGAATCGCTAAATAATATAAGTCATCATGTTC
TTGAATCTTAGAGTACCACTGAATCACAGCGATATTTTTCCGCATTAGCGTCTTTATATG
CTGACGTTTACGCTTACTGTACCGCAGCTCGCCAGCAATCGTAAACACGCGCTTGCGTGT
CTGCTCGCTCACTGATAGTGTCTTGTCATAATTTAAGACCCGCGATACCGTCGCCAATGA
GACATCTGCTCGTTGCGCAATATCTTTTAACGTTACGGCCATCGTCATTCCTCTTTTCTC
TTAATTAATTAGCACTGAATAACAAACGTGGGGCAACTGGGCGTGACACTATTCACCAAT
CATGACTCATCGGTAAATCGCGTCTGCAACACCCCGTTGCCCCACGTCTTCATTTGATTA
ACAACTTAATCGTGCATTGCTGCGACAAAACGATCAAAGGCTGCCTCACCAGCGTCATCC
CACTTAAAGACCCCAGCATCGGCCAGGACCCGGGCAAAGACGTTGCCGACCGCCGTATCA
ATGACAGTCGTCACATTGTCAGCGGTGATGGTATTAGTGGCCTTCAATTGATCAGCCCAT
GCTTGATGCATCGCGACCATTTGATTAGGCTGATCCAACAAGTATTTGCCGACTTCAGCT
AATTCGGTCTTCAACCGGGCTGGTAAAATGGCCCGGCCCATCACTTCAATCAAGCCAATA
TTTTCCTTCTTGATATGTTGAACATCCTGATGTGGATGGAAGATGCCATCTGGAAATTCA
GCAGACGTCTGATTATCACGCAAAACGATATCTAATACGTAGTCATCGCCAACTTTGCGT
GCAATCGGTGTCGTTGTGTGATGACGTGTGCCATCCGTATAAGCACGAACATCCACACTC
TCGTCCGAATAATTCTTCCACGTCTCATGAATCTTCACAGCGGCATCCGTTAATGCAACC
AAGTCCTGACTCGTCAGCCGAATCGTCGACATTGGCCATTTAACAATCCCAGCCTTAACA
CCCGCGATACCTAAGTCAATCGTGCGTGCAATTGGCGCTTTCATCATTGGAAATTCGTGC
CGACCACCTTGGTAATGCTCGTGTGACAGCATTGAACCACCAACAATTGGCAAGTCCGCA
TTACTGCCCACAAAGTAATGTGGAAATTGCTGAACAATTTCAAGTAAGTTGGTAAAAGTT
TGCCGGTTAATGACCATTGGCCGGTGCTCTTGATCCAAGAAAATCGAGTGTTCATTAAAG
TAAGCATATGGAGAATATTGGAAACCCCAAGTATTGCCACCAAGTGTTAACCGAATAATT
CGGTGATTGCTCCGTGCTGGGTAACCGAGCCGGCCTAGATAGCCTTCATTCTGCATACAT
AGTTGGCAAAGTGGATAGCCATCTTGCGGTTGGTTACGCGCTGCCGCAATCGCTTTAGGA
TCCTTTTCAGGCTTTGATAAGTTGATGGTAATCTCTAAGTCACCAAATGGCGTTTTTGCT
GGAAAGACCACGTTTTTCGCAATGGCCCGCGTCTTGATATAATCATTAGCTTTGCTTAAG
TTAAAGAAATAGTCGGTTGCCGTTTGCGGACTAACCTGATACTTATCCCAAAAACGTTGG
TTTAATACGGATGGTAACGGGGTCACAAGATCCATCAATTGATCCGCTAAGATATCTTCA
TCCGATTGGGTCGCTTCGATCTTGCCATTTTGAATCGCCGTTTTAACCAAGGCATCGGTT
AAACTTGTTAATTGGTCATCGCTTGCCGCAATCGGATCACCCTCGCCGACCAAACCAAGA
ACCCGGTTATGCACATAGATTCGATCTAATTCAGTATATTGCGAAGGCGACGCGATCACC
GCCGTGACAAATTGATCCAAACTCGTCATTATGCTTGGCCTCCCCGATCATCATAGCCGG
CCGGATGACTTTCTTTCCAAGTCCAAGCCGTCTTGATAATTTCTTTCACATCATCATACT
GAGGTGTCCAACCTAAAACAGTACGTGCCTTGTCGCTAGCTGCAACCAATGTGCTTGGAT
CACCAGCGCGGCGTGGTGCCATGATTGCTGGAATTGGCTTACCAGTGACTTCACGAGCAG
CGTCCAACATTTGTTTGTTGGAGAAGCCCGTTGAAGAACCCAAGTTAAACGCATCACTAT
CGTGCCCCTGCTTTAGGTATTCCAAGGCTAAGATATGTGCATCAGCTAAATCGACAACGT
GAACATAGTCCCGAACATTGGTCCCGTCTGGAGTGGGATAGTCGTCACCGAAGATTTGTA
ATTGATCCCGTTCACCAGCAGCTACTTGTAAAATAATTGGGACGAGATGCGTTTCTGGTG
CGTGATCTTCGCCGATGCTGCCGTCAGGCTTAGCCCCCGCCACGTTAAAGTAACGTAAGG
CCACGAATTTAATCCCGTAAGCCACATCTGACCAGTGCATGATCTTTTCCATCGCTAATT
TACTTTCACCGTATGGGTTAGTTGGCACTTGTGGGTCCGATTCCTTGATTGGCACCTGCT
TAGGTTCACCATAAGTTGCTGCGGTCGATGAGAAGACGATTTTCTTCACATCGTGCTTAG
CCACCACTTCGAGCAACTTAACCATCCCTGCCGTGTTGTTATCAAAGTACTTGAGCGGAT
CCTTCATCGATTCAGGAACAACTGAGAAAGCTGCAAAATGAATGACACCTTCAACGTTTT
CTTGGTCAAACACGGTATTCATGAACGCCGTATCCCGGACATCACCTTCATAAAAACGCG
CGTGTTCATTGACCGCTGCCCGGTGCCCCGTGACCAAGTTATCAACAACCGCCACGTCAT
ACCCCTTTGCAACCAACCGATCAACTGCGTGTGAACCGATGTAGCCGGCACCACCTAAAA
CTAAAACTGCCATTCTAATTCCTCCTCAATATTTCTCAATGCTTACTGCTAATTTGTATT
TTAAATCAATTCTTTTATTAAAGTTCCTTAGGGCCGTCTGCGATTTCAGCAATGTAGAAA
TCAGCGTCGTAACCAATCGTGTCGCGATAAACTTTACCGACGTTTTCCTTAAAGGCATCG
ACTTGGTCCTTATCAACGATGGCAATACCACAGCCACCAAAGCCAGCACCAGTCATCCGC
GCACCAAGCACGCCTGGTTGTTTCCAAGCTGTTTCGGCCAACGTATCCAATTCTTTCCCA
GTCACTTCATAGTCAAAGTGTAGTGAAACGTGGGAAGCCGTCACTAATTCGCCAAAGGTC
GTTAAATCATCGTCAGCCAAGGCCTTAGTTGCGCGAATGGCCCGTTGATTTTCGAAAACG
GCATGCCGGGCACGTTTGATTAAGGTTTCATCATTGATTAAGTAGGCCGCTTCGTCAAAC
TGGTCGTTAGTTAAATCGCCAAGTGATTTGATGTCTAACTTAGTTTGTAATCGGCGTAAT
GCTTCTTCACATTCTGAGCGGCGTTCATTATATTTTGAATCCGCCAATTCGCGCCGTTTG
TTCGTATTCATGATAACGATTACGTTGTTGCCTAACTTTACGGGTGCATAAGAATAGTCC
ATGGTGTTGGTATCAAGTAAAATTGCTTGGTCCTTTTTACCCATTCCAACAGCAAATTGA
TCCATGATCCCGGAGTTAACACCAACATAGTTGTTTTCGCACTTTTGACCAATCTTAACA
AGATCTAATTGGCTAATACCGAGATTAAAACCAGTGTTTAACATGATTCCAGTTAATAAC
TCAATTGACGCAGATGATGATAATCCAGCACCATCAGGCATGTTGCCATGGACGTAGAAA
TCAAAACCATGATCGAATTTAACGCCGGTCTTCGCGATTTCGTCCATCATTCCCTTAGGA
TAATTTGTCCAACCAGCGGCTTTGTCATATGAAAGGTCGTTGACATCAAACGTGACGATC
CCAGCATCTGGAATATTAGCTGAGTACATCCGCACTGTCGTATCTGTCCGGGGTGCATAA
ACACCATATGTCCCGATTGTAATCGCACATGGGAAAACATGGCCCCCGTTATAATCAGTA
TGTTCACCGATCAAGTTGATTCGACCAGGTGAGAAAAAGACCCGTTCTGGCTTGGTATCA
AAGGCTTCGGTAAACTCCTGTTTTAAGTCGCTAGTATTCATGAAAAATCCCTCCGTAAAT
AGTTTTACTAAAATTTTATTAGTTTGACTATAATGCTTTTTTTGATTGTTGTCAACACCC
CATAAGACCTTTAGAACAAGATTTGGTAACGCTTTACAAATTTATCACGTTATCGATTCA
AATTCTTCTTATCGCCCGTTGTCGTTGTCAGTAGTTGTTGTCATTTAGTTAAAATTTAAC
TAAAACGACATATACAAATTTAATATTTTGTTTTATGATATTTGTAAGCGTTTTATTTAT
GTAACTTTGAAAAAAGCTTCCTCATGCAAGCTAATCTTCAATGGTTAGATGACCCAGAAG
TCTTCCGGGTCAACCAATTACCTGCACATAGTGATCACCATTATTATCACGACACAGCAG
AATTCAAAACGGGTAGTCGCTTCATCAAGAGTCTCAATGGCGCTTGGCGTTTTAACTTCG
CCAAGACACCGGCTGAACGCCCAGTTGATTTTTATCAACCCGATTTCGATGCAACCGACT
TTGATACGATTCAGGTTCCCGGTCATATTGAACTAGCCGGCTATGGTCAAATTCAATACA
TTAACACGCTATACCCATGGGAAGGTAAAATTTACCGTCGCCCACCGTATACCCTCAATC
AAGATCAATTAACACCAGGCCTATTCAGTGACGCTGCGGACAACACCGTCGGCTCGTACC
TCAAAACCTTCGATCTCGACGATGCTTTTAAAGGGCAACGTATTATCATTCAGTTCCAAG
GGGTAGAAGAAGCCCTGTACGTCTGGTTAAATGGCCATTTTATTGGCTACGCTGAAGATA
GTTTCACCCCTTCTGAATTTGATTTGACGCCGTATATTCAGGACCAAGGTAACGTTTTAG
CGGTTCGGGTCTACAAACGCAGTACTGCTGCCTTTATTGAAGACCAAGATATGTTCCGTT
TCTCTGGTATTTTCCGTGACGTCAATATACTGGCGGAGCCTGCTAGCCATATTACTGATT
TGGACATCCGACCAGTTCCAAATGCCAATCTCAAAAGTGGTGAGCTCAACATCACTACTA
AAGTAACCGGCGAACCAGCCACTTTAGCGCTGACCGTTAAAGACCATGACGGGCGAGTAC
TGACGAGTCAAACGCAAACCGGTAGTGGCAGTGTAACCTTTGATACCATGTTATTCGACC
AACTGCACTTGTGGTCACCACAAACGCCGTATCTCTATCAATTGACAATTGAAGTTTACG
ATGCTGATCACCAACTCTTGGAAGTCGTCCCATATCAGTTTGGGTTCCGGACGGTCGAGC
TGCGCGATGACAAAGTCATTTACGTCAACAATAAACGGTTGGTGATCAACGGGGTTAACC
GGCACGAATGGAACGCCCACACCGGTCGCGTTATCAGTATGGCTGATATGCGCGCTGATA
TCCAAACCATGTTAGCTAACAATATCAATGCCGATCGGACCTGCCATTATCCTGATCAAT
TACCTTGGTATCAATTATGTGACGAGGCCGGTATCTACCTAATGGCCGAAAACAACCTCG
AATCGCACGGGTCATGGCAAAAGATAGGGGCTATCGAGCCTTCTTACAATGTTCCTGGCG
ATAATCCACACTGGTTAGCAGCGGTGATCGACCGGGCCCGTTCAAACTACGAATGGTTTA
AAAACCACCCATCGATCATTTTTTGGTCACTTGGCAATGAATCGTATGCTGGCGAAGATA
TCGCGGCGATGCAGGCTTTTTATAAAGAACACGATGATTCACGACTCGTCCACTACGAAG
GTGTTGTCCACACACCAGAATTAAAAGATCGCATTTCTGATGTTGAAAGTCGGATGTACG
AAAAACCCCAAAATATTGTAGCTTACTTGGAAGATAACCCAACCAAACCTTTCCTAGATT
GTGAATATATGCATGACATGGGGAATTCTCTGGGCGGTATGCAATCATATAATGATTTGA
TCGACAAGTATCCGATGTATCAAGGTGGCTTTATTTGGGACTTTATTGATCAAGCCCTCT
TCGTTCATGACCCAATTACCGACCAAGACGTGCTCCGGTATGGCGGTGATTTCGACGAAC
GCCACTCCGATTATGAATTCTCCGGTGACGGCTTAATGTTTGCTGACCGGACGCCAAAAC
CAGCAATGCAAGAGGTGAAATATTATTATGGCTTACACAAATAATCAACTACACGTTATT
TACGGCGACGGGAGTTTAGGACTACAGGGGGCTAATTTCCACTACCTCTTTAGCTACGAA
CGTGGCGGACTTGAATCACTCGTCGTCAACGATAAAGAATGGCTCTATCGTACACCCACG
CCCATCTTTTGGCGGGCGACAACCGATAATGATCACGGTAGCGGCTTTTCAGTCAAATCC
GCACAGTGGTACGCGGCCGATAAGTTCTCAACTTGTCAAGATATCGAATTGACGGTTGAC
GACCAACCAGTCACACCGTTACCAATCGCGCCACTCAATAACAAATACACGGATCACGAA
ATCGCCACGAAAGTCTCACTGGCTTACCACTTCGTTACCACGACCGTTCCTAGTACCATC
GTCACAGTGACTTATACGGTGACAGCAGACGGTCAGATCAATATCGCCACCCATTATAGC
GGTCAGTCTGATTTGCCAGAGCTACCCGCATTTGGTCTGCGGTTTATCATACCAACTACC
GCGACCGGCTTCGACTATACCGGTTTGTCCGGTGAGACTTATCCTGACCGGCTGGCCGGC
GCAACGCACGGGCGATTCCACGTTGACAGTCTGCCAGTCACACCATACTTGGTCCCACAA
GAATGCGGCATGCACATGCAAACTGAACAAGTGACAGTAACGCGATCAACAACACAAAAT
AACGCTGACCACGACAACACACCGTTCAGTTTGACATTTAGCCAAGCCGATGCACCATTC
GCCTTCAGCTGCCTTCCCTATACCGCCGCTGAACTAGAAAACGCAACGCACATGGAAGAA
TTACCATTAGCACGGCGAACGGTCTTATCAATCTACGGTGCCGTTCGTGGGGTCGGTGGC
ATTGATAGTTGGGGAACAGACGTAGAATCCCCATATCATATCCCCGCTGATCAAGACATT
GACTTCAGCTTTAATATTCATTTCTAAAAGTTATTTTGATTTCAAAAGAACGCTCCGGCG
AGTTATTTGCCAGAGCGTTCTTTTAGATTAACGATGATTAAGTTTTAATATGTTTAATGG
CTGAGCTTAGTCCTTAGCCTTGAAGTAATGTACCTGCGCCGTAAAGTCACTAGTTTGAAC
GGGAGTCGTGTAGAGTCCTAGTTGCATCAATTCATCGCCACCGTACATTTTGTTTGTGTC
GGTCTCAACGTAGGTCTGCTGAGGGTCTAATCCCGCTAACTTCGTAATATGCGGTTCTGG
TTGAACTGCACCTAAAATAACGAAGGTGAACAGCAAGGCTTCTTTCTGATCCGGACTAAC
AAACATCCACGCCACCGTATTAGATTCAAACGGGCTCTCTAATCGATAAAAGGTCCCGTA
TTGAACTAACTCACGGTGCTGCTTATAGAAGGCAACCTGTCTTTTAACGGCCTGCTTGTC
CGCATCACTTAGTTGGGCCGCGTCCAGTTCATAGCCCAAGGTACCACTCATTGCCACAGC
ACCACGCATCTTCATCGACGTCGACCGTCCTAACAATTCATCCGGGCTCGTCCCAACATG
GGCGGTAATTGCAGAAATTGGATAAACGAGTGAAGTCCCATATTGAATTTTGAGCCGTTC
AATCGGGTCATTATTATCTGATGGCCAACTCTGTGGCATATAATACATTAAACCAGCATC
GAAGCGGCCACCACCGCCAGAGCAGCCTTCAAATAAGATCTGTGGATAGCGTTTTGTTAA
ACGCTCCATCAAGTCATATACACCTAAGATAAAGCGGTGGCTAGTCTCACCTTCATGAGT
TGAATCGCTATGTGGCGAATAGACTTCCGTTAAATTGCGGTTCATATCCCACTTGATATA
ATCAATGGGCACCTTATCAAGAACTGCGGTCATTTGTTCAAAAATATTGTCGACAACATC
CGGCTGGCTGAAATCAAGCACGTATTCGTTCCGCGATAACGTTCGGCCACGATCTGGTAC
GCCTAACACCCAATCAGGATGGGCCCGATAAAGATCGGAATCGGCTGAAATATTTTCTGG
CTCGAACCAAAGTCCGAACCGCATCCCTTTATCATGGGTCCGTTTGCTAATATCCGCTAA
GCCTCCTGGTAACTTGTTCCGATTAACAAACCAGTCACCTAGCGAACTATTATCATCATT
TCGATGACCGAACCAACCGTCATCCAGCACAAACATTTCGATACCCAGTGGTGCTGCAGC
ATCCAAGATTGATTGAATCTTGTCCGCATCAAAGTCAAAGTAAGTGGCTTCCCAATTGTT
GATCAAGATCGGCCGGAGATCGTGCTTGTAACGACTGCGCGCCACGCGATCACGCAATAA
ATCATGGTAGGTTTGCGACATACCATTAAGCCCCGTCGTCGAGTATACCATTGCAACCTC
GGGCGTTTGGAAACTATCACCGGCCGCCAATCGCCAGTCGAAGTTATATTCATTGATTCC
CATAGTCAAGCGTGTCTGACCAATTGGGTCCCGTTCTAAACTAATCTGGTGGTTACCTGA
ATAAATTAGCAATGCACCAAGGACATTGCCTTGAAATTCATTAGTATTCGGATCAGCTAG
CGCCACAAAAGGATTCATATGATGAGAACTAGCACCCCGGCGACTTTCAAACTGAGTTAC
ACCTCGATGGAGCCGTTCACGACTAAGCTGGCGCTCACGCGCATAGCTTCCAGGTAACGA
AATGGCATCTAATGGTTGGGTTGGCAGGTCCATCTGCATCGAAGCCACTTTTTCTAAGTC
GACAGCTTGCTCACTGTGATTAATCAACCGGGCGCTACGCGTGATAACTGGCCGATCCCG
ATAAATCGTGTAGGCTAATTCGAGTGTCACGCCTAATGTGGCATCTCGCAACGTCACAAT
CAAAGTTTGGGCTTCATCCTCATCTTCGACATAAGTTTGTGGCAAGCCCTTAAGTTCCGG
CTTACCGGCTTCCATCCGGCAGTCTTGATAACGAAAATCAACTGTTCGAGCACCATTGGC
CGCGCGAATAATCGCGGCGGGTTGCCGATAATCACCGGTATTATTGCCACCGTATTCCTG
TGGCAAATCATCCTTGGAATAGCTTCGATCAGTCGTATTCGGTAAATTTCCTGAGAACCC
CCGATCAATCCGGGGATACTGACGCTCGCCATGATAACCCCGAACCCGTGGGCCAAAGTA
CAAGTGGGCCAACACGTTGCCTGTCTCCACACCTAGAATATACGAGATTTGATCGTTATG
CAGGTGAAAGACAAGTTGTTCTTCATCAATGTCAATTAAAGTTTGCTGCAACGTTACTGC
CATAGTAAAATCTTCCCTTCGCTATTAGCTTTTTCTATTTCAGTTCACAAAAAGAGCTAT
TAATGGCTCGCGTCATTAATAGCTCTCCTCGTAAAGAAACTATGCTTGATCCGGTAAGCC
GACATTCGATAATTGTAAGACATCTTCATCATCTTGTACGGTGGTTCCTGAATCTTTAAG
ATACGTAAATTCGGGCATATCTTTATAGTTTGTAATAACGACCATAACGGTATCATCCAA
GCCAGCCTTTTCAATTGCTGGTTGCCAGAATTCAATTAATTCTTGCCCTTGTGTGACATG
GTCGCCCTTCTCAACGTACTGAACAAATCCAGTCCCACGCATCTTAACGGTACCAATCCC
AATATGAATTAGCGTTAAAATACCGGTATCAGAACGTAATCCGATTGCATGGCGAGTCGG
GAAGGTTGCAACAACTTCACCATCAAATGGTGCGACAACGCGACCATCCGTTGGTTTAAT
GGCGAAACCTTTACCCATACTACCGTTTGCGAAGGATTCGTCAGCAACATCTTTCAAACT
GATCAGTTCCCCGGCAACAGGATCTTGATAAACCGTCGTCTTGGCATCGGCTTCTGCTTC
AACTTCAGCAGGAGTTTCATCGGTTTCCAAGTGAGTTGACCATGTTTTTTCAAGTTCATC
AACGATTTGTGCATGCATCTTTTCATCCAACTTAACCTTCTTGAAGAAGATGAATGTCCC
AATCAAAATCATAGCAGCAGGTACACCGAACATCATTAACTTGAAGATGGCAGCGCCATG
GCTAGTCACATCACCAGCAGTGGCACCAGCTGTCATCCCAGCCCAAACAGCAGTTAAACC
AACGACACCGTTAGAAATCGCACCGCCTAATTTATCAAGTAAAGGCCGAACGGATAAGGT
TAAAGATTCGTCACGATGACCAAACTTCAACTGCCCATATTCAACAGAATCACTCAAAAC
CATCAAAACAACTAAGAAAATTAATGGTTGTGGAATGTAGAATAATACGGCCGCTGTTAA
GATCATTGGTAATGACTGACCAGCAAAGGCAAACATGATCAGTGAAATCATCATAATAAC
AACTGCTAAGAAGAACACTTTACGACGACTAAACTTCTTTGCTAATGGTGGGAAGGCCAA
TACGGCAAAAATCCCAATAATCGCATTTAGCGAACCTAATAGTGTAAACTTCGAGGCGTC
ACCCAAAATATAGGTGAAGTAGTAAAGTTCTAGTGAGTTCGTAATGGCAATCCCTGCCGT
GTAGATCCCATACATCAATGATAACCACATCAATTGATCGTTACGGGCTAACACTTTGAA
GACTTCCTTGAAGCTAGTCTTTTCAGTGTTCTTACGCAATTCAGAATCATTTTCTTTGGT
TCCAAGTGCAACAACTAAGGCAGAAATCAAAGCGATTGCGGCAATAATGATCCCAAATGC
CATCCAACCACGGTTGTCACCTTGACCACTGTTAGACTTAACAGAAAACATTAAGACGAT
TGGCATAACGATAACCCCAACGATGTTGGCACCAATGTTAGAGCCAACCCGGGCGTATGT
CGCCGTCTTTTCACGTTCTGCTGAGTCAAATGAGATTGCTGGAATCATTGACCAAAAACC
AACATCTTTAAACGAATAGAAAATATCCATTGTAATGTATAAAATTGCAAAAATAATTAA
ATATAGAATGGGGTTTGAGGTATTTAATCCGCCTAAACTCGTGAATAAAAGTGCTAAAAC
GACGGAACCTATAATCCCACCAATGACAATCCATGGTTTAAAGTGGCCCCACCGAGAATT
AGTATTATCAATGGCATTCCCGATCAAAGGATCAATTGCTAGTTCCACGAACCGCAAACC
TGCAATAATCATGGTGATCCAAGCAATCATTTGTGTTCCTTGAGCCCCACTACTCTTATC
GAACAAGTGGGAGGTCACAAACATAATAAAATAAGTTGATAAAGTGGCATAGAAGGCATC
GTGCCCAAACGCCCCAAACGAATAAGCAAGTCGCGATGTTATTTTACGACCTTTACTCTG
CTTTGTCTCTGCATCCAT
> SEQ ID NO: 2 lacA of Lactiplantibacillus plantarum
DSM33967
ATGAATAAGTTAGAACTGGTTAAAGGCTTTTTACATGGTGGTGACTACAATCCTGATCAG
TGGTTAAAACGACCGGATATTTTGGACCGGGATTATCAGTTGATGGATCAGTCGAAGATT
AACACGGTTACTGTTGGTGTTTTTGCTTGGGGACGGCTTGAACCGGAAGAAGGTGTTTAT
GATTTTGCATGGCTTGATGATGTTTTCGATAAAATGAATAGTCGAAATGGCCACGTGATT
TTAGCAACACCAAGTGGCGCACGGCCACGATGGATGAGTCAGAAATATCCTGAGGTTAAT
CGAGTTGATTCTGAAGGTCGTCGTCATACTCATGGTTTCCGACATAATCATTGCTATTCG
TCCCCGATTTATCGAAGAAAGGTACATGAAATCAATATGCGCTTGGCAGAGCGATATGGA
GACAATTCAGCGCTTGTTTTGTGGCATGTCTCTAATGAATATTCTGGAGAGTGTTACTGC
CCTTTGTGTCAACAAAAGTGGCGGGAGTGGGTCCAGCACAAATATAAGACACTTGAAGCA
TTAGATGACGCGTGGTGCTCGAGCGTCTGGAGTGGTTTATACAGTGATTGGTCACAATTG
TTGCCACCATCACTATTGGGTGATAGCAAAGTTCATGGCATGGATTTAGACTGGCACCGG
TTTGTGTCAGATATGACGATTGATTTTTATCAGCATGAGATTCAGCCTTTAAAGAAAGTA
ACGCCCAATATTCCCGTAACAACTAATTTTATGGCTGAGAGCTTAAAAGAAGGGCAGTAT
ACACCGCTGACAGGGTTAGATTATAGTAGATTTGCAAAGTCTTTAGATATAATTAGTTGG
GATAGTTATCCTAGTTGGCATAATCGCTATGAAATTACTGCGGAAACTGCCATGAAAACC
GCGTATGTTCATGATCAGTTTTGGTCTTTAAAACAACAACCATTTTTGGTCATGGAGTCA
ACACCAAGCTGTGTTAACTGGGGACAGTATAATAAAGCCAAGCAACCTGGCATGCATCGG
CTGAGTTCATTTCAACAGATTGCGCATGGCTCAGATAGTACGTTATACTTCCAGATTAGA
CAGGCACAAGGTAATTCAGAAAAGTATCATGGTGCCGTAATTGGACATGATGATAGCTCA
GATAGCCGCGTTTTTAAAGACGTGCAACAATATGGACAAGATTTGGAAAAAATTGTAGAA
GTTAAAGGGGCTATTAAGCGTGTAAGAGTTGCTATCCTCTTTGACTGGGATAGCAACTGG
GCCCTGAACCGTGGTGGCGGCTTTGGTCGGCCAACTAGATTGGGAATACAAGTACTTCAA
AAACATTATGCAAATTTTTGGCACAAAGATATTGAGTGTAATGTTATAACGAGTGATATG
GACTTTTCTCAATATGATTTGTTAATTGCACCGATGCTTTATTTAATACGACAAGATACG
ATGAAAAAATTGCAAAAATTTGTTCAAAGTGGTGGCACATTGGTATCTAGCTATTTTTCA
GGTATGGTTGATAGCAATGATCGTGTGAATTTAGATGGCTGGCCTAAACCGCTACAGGAT
ATTTTTGGAATTGAGCCAAAAGAATTGGATACTTTATTACCAGGCGAGCACTACCATGTT
AGTTTTAATCAAAAAGAGTATGAAACTACCGATTATGATCAAATAATGAAAGTTAAAAAC
GCATCAGTGCTGGGATACTACACAGACAACTTCTATGTTGGAACACCAGCAATTACACGG
CATCAGTATGGTAAAGGAACGGCCTATTATATTGGTGCACGCTTGTCTGCGGAATTCATT
GAAGACTTTTATGATGACATTATTCAAAGCCTTAATTTAGATAATAGTTTTGTCGAATTG
AGCAATCCGGTAGTATCAGTTCAAACTCGTTATAATGAGGATTATGCCTATCACTTTGTT
ATGAATTTTTCTGAACAAGTACAAATGGTTAATTTTCGACGTCCATCCTTTGATCTGCTT
ACACAGCGAGAAGTTACGAAATCAGTGCAAATTGATAAGTATGGGGTCATGATTCTGAAG
GAAAGACAGAACAATACGTTGCACTAG
> SEQ ID NO: 3 galR of Lactiplantibacillus plantarum
DSM33967
ATGGCCGTAACGTTAAAAGATATTGCGCAACGAGCAGATGTCTCATTGGCGACGGTATCG
CGGGTCTTAAATTATGACAAGACACTATCAGTGAGCGAGCAGACACGCAAGCGCGTGTTT
ACGATTGCTGGCGAGCTGCGGTACAGTAAGCGTAAACGTCAGCATATAAAGACGCTAATG
CGGAAAAATATCGCTGTGATTCAGTGGTACTCTAAGATTCAAGAACATGATGACTTATAT
TATTTAGCGATTCGTAAAGGAATTGAGCATCAAGCTCAAGTGTGTGGTTTTACGACGACT
AGTATCTTCCAGAATAATTTAGATCAGGTTGGCGATGATATTGATGCAATCGTGGCGGTG
GGAAAATTTAGCCCTGTCCAAGTACAACAGTTGAGTCGTTTGTCAAGCCAGCTTGTTTTT
GTTGATGACGATCACTTTGCTCAGGGATTCAGCAGTGTGGTGACTGATTTTACGTTTGCT
ACTAACCGCGTCGTTGATTATTTTTGGCGGCGTGGTATCCGAGATATCGGTATGATTTAT
GGCAAAGAGTGGTCCACTGATCAGCAGGTGGAGATTCCCAATCAACGTCAGCAAAGCTTT
GAACAGGCGTTGCAGCGGCATCATGCCTATCATGCTGAGTATGTATTAGCTGGTGATTAC
ACTAGTCAATCTGGTTATCTGATGATGAAACGGGCCATTGAAATGTTAGGTGACCGCTTA
CCACACGCTTTCTTTATCGCTAATGATCCGATGGCAGCCGGTGCGCTCAAAGCGTTACAG
CAAGCTGATATTGCTGTGCCACAGCGGGTCAAGTTGTTTAGTTTCAATAACACGACGCTG
GCCGAATATGTGTATCCAGAGATTAGCTCGGTCAATGTTGAGACTGAACTGATGGGAAAG
ACGGCCATCGATTTGTTGGTGAGCCGATTCCAAGATGATCGTCAAGCAGCCCAGCGCATT
GAACTGGGAACGACGTTGGTAGAACGTGAAAGCACCAAATGA
> SEQ ID NO: 4 galT of Lactiplantibacillus plantarum
DSM33967
ATGACGAGTTTGGATCAATTTGTCACGGCGGTGATCGCGTCGCCTTCGCAATATACTGAA
TTAGATCGAATCTATGTGCATAACCGGGTTCTTGGTTTGGTCGGCGAGGGTGATCCGATT
GCGGCAAGCGATGACCAATTAACAAGTTTAACCGATGCCTTGGTTAAAACGGCGATTCAA
AATGGCAAGATCGAAGCGACCCAATCGGATGAAGATATCTTAGCGGATCAATTGATGGAT
CTTGTGACCCCGTTACCATCCGTATTAAACCAACGTTTTTGGGATAAGTATCAGGTTAGT
CCGCAAACGGCAACCGACTATTTCTTTAACTTAAGCAAAGCTAATGATTATATCAAGACG
CGGGCCATTGCGAAAAACGTGGTCTTTCCAGCAAAAACGCCATTTGGTGACTTAGAGATT
ACCATCAACTTATCAAAGCCTGAAAAGGATCCTAAAGCGATTGCGGCAGCGCGTAACCAA
CCGCAAGATGGCTATCCACTTTGCCAACTATGTATGCAGAATGAAGGCTATCTAGGCCGG
CTCGGTTACCCAGCACGGAGCAATCACCGAATTATTCGGTTAACACTTGGTGGCAATACT
TGGGGTTTCCAATATTCTCCATATGCTTACTTTAATGAACACTCGATTTTCTTGGATCAA
GAGCACCGGCCAATGGTCATTAACCGGCAAACTTTTACCAACTTACTTGAAATTGTTCAG
CAATTTCCACATTACTTTGTGGGCAGTAATGCGGACTTGCCAATTGTTGGTGGTTCAATG
CTGTCACACGAGCATTACCAAGGTGGTCGGCACGAATTTCCAATGATGAAAGCGCCAATT
GCACGCACGATTGACTTAGGTATCGCGGGTGTTAAGGCTGGGATTGTTAAATGGCCAATG
TCGACGATTCGGCTGACGAGTCAGGACTTGGTTGCATTAACGGATGCCGCTGTGAAGATT
CATGAGACGTGGAAGAATTATTCGGACGAGAGTGTGGATGTTCGTGCTTATACGGATGGC
ACACGTCATCACACAACGACACCGATTGCACGCAAAGTTGGCGATGACTACGTATTAGAT
ATCGTTTTGCGTGATAATCAGACGTCTGCTGAATTTCCAGATGGCATCTTCCATCCACAT
CAGGATGTTCAACATATCAAGAAGGAAAATATTGGCTTGATTGAAGTGATGGGCCGGGCC
ATTTTACCAGCCCGGTTGAAGACCGAATTAGCTGAAGTCGGCAAATACTTGTTGGATCAG
CCTAATCAAATGGTCGCGATGCATCAAGCATGGGCTGATCAATTGAAGGCCACTAATACC
ATCACCGCTGACAATGTGACGACTGTCATTGATACGGCGGTCGGCAACGTCTTTGCCCGG
GTCCTGGCCGATGCTGGGGTCTTTAAGTGGGATGACGCTGGTGAGGCAGCCTTTGATCGT
TTTGTCGCAGCAATGCACGATTAA
> SEQ ID NO: 5 galE2 of Lactiplantibacillus plantarum
DSM33967
ATGGCAGTTTTAGTTTTAGGTGGTGCCGGCTACATCGGTTCACACGCAGTTGATCGGTTG
GTTGCAAAGGGGTATGACGTGGCGGTTGTTGATAACTTGGTCACGGGGCACCGGGCAGCG
GTCAATGAACACGCGCGTTTTTATGAAGGTGATGTCCGGGATACGGCGTTCATGAATACC
GTGTTTGACCAAGAAAACGTTGAAGGTGTCATTCATTTTGCAGCTTTCTCAGTTGTTCCT
GAATCGATGAAGGATCCGCTCAAGTACTTTGATAACAACACGGCAGGGATGGTTAAGTTG
CTCGAAGTGGTGGCTAAGCACGATGTGAAGAAAATCGTCTTCTCATCGACCGCAGCAACT
TATGGTGAACCTAAGCAGGTGCCAATCAAGGAATCGGACCCACAAGTGCCAACTAACCCA
TACGGTGAAAGTAAATTAGCGATGGAAAAGATCATGCACTGGTCAGATGTGGCTTACGGG
ATTAAATTCGTGGCCTTACGTTACTTTAACGTGGCGGGGGCTAAGCCTGACGGCAGCATC
GGCGAAGATCACGCACCAGAAACGCATCTCGTCCCAATTATTTTACAAGTAGCTGCTGGT
GAACGGGATCAATTACAAATCTTCGGTGACGACTATCCCACTCCAGACGGGACCAATGTT
CGGGACTATGTTCACGTTGTCGATTTAGCTGATGCACATATCTTAGCCTTGGAATACCTA
AAGCAGGGGCACGATAGTGATGCGTTTAACTTGGGTTCTTCAACGGGCTTCTCCAACAAA
CAAATGTTGGACGCTGCTCGTGAAGTCACTGGTAAGCCAATTCCAGCAATCATGGCACCA
CGCCGCGCTGGTGATCCAAGCACATTGGTTGCAGCTAGCGACAAGGCACGTACTGTTTTA
GGTTGGACACCTCAGTATGATGATGTGAAAGAAATTATCAAGACGGCTTGGACTTGGAAA
GAAAGTCATCCGGCCGGCTATGATGATCGGGGAGGCCAAGCATAA
> SEQ ID NO: 6 galK of Lactiplantibacillus plantarum
DSM33967
ATGAATACTAGCGACTTAAAACAGGAGTTTACCGAAGCCTTTGATACCAAGCCAGAACGG
GTCTTTTTCTCACCTGGTCGAATCAACTTGATCGGTGAACATACTGATTATAACGGGGGC
CATGTTTTCCCATGTGCGATTACAATCGGGACATATGGTGTTTATGCACCCCGGACAGAT
ACGACAGTGCGGATGTACTCAGCTAATATTCCAGATGCTGGGATCGTCACGTTTGATGTC
AACGACCTTTCATATGACAAAGCCGCTGGTTGGACAAATTATCCTAAGGGAATGATGGAC
GAAATCGCGAAGACCGGCGTTAAATTCGATCATGGTTTTGATTTCTACGTCCATGGCAAC
ATGCCTGATGGTGCTGGATTATCATCATCTGCGTCAATTGAGTTATTAACTGGAATCATG
TTAAACACTGGTTTTAATCTCGGTATTAGCCAATTAGATCTTGTTAAGATTGGTCAAAAG
TGCGAAAACAACTATGTTGGTGTTAACTCCGGGATCATGGATCAATTTGCTGTTGGAATG
GGTAAAAAGGACCAAGCAATTTTACTTGATACCAACACCATGGACTATTCTTATGCACCC
GTAAAGTTAGGCAACAACGTAATCGTTATCATGAATACGAACAAACGGCGCGAATTGGCG
GATTCAAAATATAATGAACGCCGCTCAGAATGTGAAGAAGCATTACGCCGATTACAAACT
AAGTTAGACATCAAATCACTTGGCGATTTAACTAACGACCAGTTTGACGAAGCGGCCTAC
TTAATCAATGATGAAACCTTAATCAAACGTGCCCGGCATGCCGTTTTCGAAAATCAACGG
GCCATTCGCGCAACTAAGGCCTTGGCTGACGATGATTTAACGACCTTTGGCGAATTAGTG
ACGGCTTCCCACGTTTCACTACACTTTGACTATGAAGTGACTGGGAAAGAATTGGATACG
TTGGCCGAAACAGCTTGGAAACAACCAGGCGTGCTTGGTGCGCGGATGACTGGTGCTGGC
TTTGGTGGCTGTGGTATTGCCATCGTTGATAAGGACCAAGTCGATGCCTTTAAGGAAAAC
GTCGGTAAAGTTTATCGCGACACGATTGGTTACGACGCTGATTTCTACATTGCTGAAATC
GCAGACGGCCCTAAGGAACTTTAA
> SEQ ID NO: 7 lacL of Lactiplantibacillus plantarum
DSM33967
ATGCAAGCTAATCTTCAATGGTTAGATGACCCAGAAGTCTTCCGGGTCAACCAATTACCT
GCACATAGTGATCACCATTATTATCACGACACAGCAGAATTCAAAACGGGTAGTCGCTTC
ATCAAGAGTCTCAATGGCGCTTGGCGTTTTAACTTCGCCAAGACACCGGCTGAACGCCCA
GTTGATTTTTATCAACCCGATTTCGATGCAACCGACTTTGATACGATTCAGGTTCCCGGT
CATATTGAACTAGCCGGCTATGGTCAAATTCAATACATTAACACGCTATACCCATGGGAA
GGTAAAATTTACCGTCGCCCACCGTATACCCTCAATCAAGATCAATTAACACCAGGCCTA
TTCAGTGACGCTGCGGACAACACCGTCGGCTCGTACCTCAAAACCTTCGATCTCGACGAT
GCTTTTAAAGGGCAACGTATTATCATTCAGTTCCAAGGGGTAGAAGAAGCCCTGTACGTC
TGGTTAAATGGCCATTTTATTGGCTACGCTGAAGATAGTTTCACCCCTTCTGAATTTGAT
TTGACGCCGTATATTCAGGACCAAGGTAACGTTTTAGCGGTTCGGGTCTACAAACGCAGT
ACTGCTGCCTTTATTGAAGACCAAGATATGTTCCGTTTCTCTGGTATTTTCCGTGACGTC
AATATACTGGCGGAGCCTGCTAGCCATATTACTGATTTGGACATCCGACCAGTTCCAAAT
GCCAATCTCAAAAGTGGTGAGCTCAACATCACTACTAAAGTAACCGGCGAACCAGCCACT
TTAGCGCTGACCGTTAAAGACCATGACGGGCGAGTACTGACGAGTCAAACGCAAACCGGT
AGTGGCAGTGTAACCTTTGATACCATGTTATTCGACCAACTGCACTTGTGGTCACCACAA
ACGCCGTATCTCTATCAATTGACAATTGAAGTTTACGATGCTGATCACCAACTCTTGGAA
GTCGTCCCATATCAGTTTGGGTTCCGGACGGTCGAGCTGCGCGATGACAAAGTCATTTAC
GTCAACAATAAACGGTTGGTGATCAACGGGGTTAACCGGCACGAATGGAACGCCCACACC
GGTCGCGTTATCAGTATGGCTGATATGCGCGCTGATATCCAAACCATGTTAGCTAACAAT
ATCAATGCCGATCGGACCTGCCATTATCCTGATCAATTACCTTGGTATCAATTATGTGAC
GAGGCCGGTATCTACCTAATGGCCGAAAACAACCTCGAATCGCACGGGTCATGGCAAAAG
ATAGGGGCTATCGAGCCTTCTTACAATGTTCCTGGCGATAATCCACACTGGTTAGCAGCG
GTGATCGACCGGGCCCGTTCAAACTACGAATGGTTTAAAAACCACCCATCGATCATTTTT
TGGTCACTTGGCAATGAATCGTATGCTGGCGAAGATATCGCGGCGATGCAGGCTTTTTAT
AAAGAACACGATGATTCACGACTCGTCCACTACGAAGGTGTTGTCCACACACCAGAATTA
AAAGATCGCATTTCTGATGTTGAAAGTCGGATGTACGAAAAACCCCAAAATATTGTAGCT
TACTTGGAAGATAACCCAACCAAACCTTTCCTAGATTGTGAATATATGCATGACATGGGG
AATTCTCTGGGCGGTATGCAATCATATAATGATTTGATCGACAAGTATCCGATGTATCAA
GGTGGCTTTATTTGGGACTTTATTGATCAAGCCCTCTTCGTTCATGACCCAATTACCGAC
CAAGACGTGCTCCGGTATGGCGGTGATTTCGACGAACGCCACTCCGATTATGAATTCTCC
GGTGACGGCTTAATGTTTGCTGACCGGACGCCAAAACCAGCAATGCAAGAGGTGAAATAT
TATTATGGCTTACACAAATAA
> SEQ ID NO: 8 lacM of Lactiplantibacillus plantarum
DSM33967
ATGGCTTACACAAATAATCAACTACACGTTATTTACGGCGACGGGAGTTTAGGACTACAG
GGGGCTAATTTCCACTACCTCTTTAGCTACGAACGTGGCGGACTTGAATCACTCGTCGTC
AACGATAAAGAATGGCTCTATCGTACACCCACGCCCATCTTTTGGCGGGCGACAACCGAT
AATGATCACGGTAGCGGCTTTTCAGTCAAATCCGCACAGTGGTACGCGGCCGATAAGTTC
TCAACTTGTCAAGATATCGAATTGACGGTTGACGACCAACCAGTCACACCGTTACCAATC
GCGCCACTCAATAACAAATACACGGATCACGAAATCGCCACGAAAGTCTCACTGGCTTAC
CACTTCGTTACCACGACCGTTCCTAGTACCATCGTCACAGTGACTTATACGGTGACAGCA
GACGGTCAGATCAATATCGCCACCCATTATAGCGGTCAGTCTGATTTGCCAGAGCTACCC
GCATTTGGTCTGCGGTTTATCATACCAACTACCGCGACCGGCTTCGACTATACCGGTTTG
TCCGGTGAGACTTATCCTGACCGGCTGGCCGGCGCAACGCACGGGCGATTCCACGTTGAC
AGTCTGCCAGTCACACCATACTTGGTCCCACAAGAATGCGGCATGCACATGCAAACTGAA
CAAGTGACAGTAACGCGATCAACAACACAAAATAACGCTGACCACGACAACACACCGTTC
AGTTTGACATTTAGCCAAGCCGATGCACCATTCGCCTTCAGCTGCCTTCCCTATACCGCC
GCTGAACTAGAAAACGCAACGCACATGGAAGAATTACCATTAGCACGGCGAACGGTCTTA
TCAATCTACGGTGCCGTTCGTGGGGTCGGTGGCATTGATAGTTGGGGAACAGACGTAGAA
TCCCCATATCATATCCCCGCTGATCAAGACATTGACTTCAGCTTTAATATTCATTTCTAA
> SEQ ID NO: 9 lacS of Lactiplantibacillus plantarum
DSM33967
ATGGATGCAGAGACAAAGCAGAGTAAAGGTCGTAAAATAACATCGCGACTTGCTTATTCG
TTTGGGGCGTTTGGGCACGATGCCTTCTATGCCACTTTATCAACTTATTTTATTATGTTT
GTGACCTCCCACTTGTTCGATAAGAGTAGTGGGGCTCAAGGAACACAAATGATTGCTTGG
ATCACCATGATTATTGCAGGTTTGCGGTTCGTGGAACTAGCAATTGATCCTTTGATCGGG
AATGCCATTGATAATACTAATTCTCGGTGGGGCCACTTTAAACCATGGATTGTCATTGGT
GGGATTATAGGTTCCGTCGTTTTAGCACTTTTATTCACGAGTTTAGGCGGATTAAATACC
TCAAACCCCATTCTATATTTAATTATTTTTGCAATTTTATACATTACAATGGATATTTTC
TATTCGTTTAAAGATGTTGGTTTTTGGTCAATGATTCCAGCAATCTCATTTGACTCAGCA
GAACGTGAAAAGACGGCGACATACGCCCGGGTTGGCTCTAACATTGGTGCCAACATCGTT
GGGGTTATCGTTATGCCAATCGTCTTAATGTTTTCTGTTAAGTCTAACAGTGGTCAAGGT
GACAACCGTGGTTGGATGGCATTTGGGATCATTATTGCCGCAATCGCTTTGATTTCTGCC
TTAGTTGTTGCACTTGGAACCAAAGAAAATGATTCTGAATTGCGTAAGAACACTGAAAAG
ACTAGCTTCAAGGAAGTCTTCAAAGTGTTAGCCCGTAACGATCAATTGATGTGGTTATCA
TTGATGTATGGGATCTACACGGCAGGGATTGCCATTACGAACTCACTAGAACTTTACTAC
TTCACCTATATTTTGGGTGACGCCTCGAAGTTTACACTATTAGGTTCGCTAAATGCGATT
ATTGGGATTTTTGCCGTATTGGCCTTCCCACCATTAGCAAAGAAGTTTAGTCGTCGTAAA
GTGTTCTTCTTAGCAGTTGTTATTATGATGATTTCACTGATCATGTTTGCCTTTGCTGGT
CAGTCATTACCAATGATCTTAACAGCGGCCGTATTATTCTACATTCCACAACCATTAATT
TTCTTAGTTGTTTTGATGGTTTTGAGTGATTCTGTTGAATATGGGCAGTTGAAGTTTGGT
CATCGTGACGAATCTTTAACCTTATCCGTTCGGCCTTTACTTGATAAATTAGGCGGTGCG
ATTTCTAACGGTGTCGTTGGTTTAACTGCTGTTTGGGCTGGGATGACAGCTGGTGCCACT
GCTGGTGATGTGACTAGCCATGGCGCTGCCATCTTCAAGTTAATGATGTTCGGTGTACCT
GCTGCTATGATTTTGATTGGGACATTCATCTTCTTCAAGAAGGTTAAGTTGGATGAAAAG
ATGCATGCACAAATCGTTGATGAACTTGAAAAAACATGGTCAACTCACTTGGAAACCGAT
GAAACTCCTGCTGAAGTTGAAGCAGAAGCCGATGCCAAGACGACGGTTTATCAAGATCCT
GTTGCCGGGGAACTGATCAGTTTGAAAGATGTTGCTGACGAATCCTTCGCAAACGGTAGT
ATGGGTAAAGGTTTCGCCATTAAACCAACGGATGGTCGCGTTGTCGCACCATTTGATGGT
GAAGTTGTTGCAACCTTCCCGACTCGCCATGCAATCGGATTACGTTCTGATACCGGTATT
TTAACGCTAATTCATATTGGGATTGGTACCGTTAAGATGCGTGGGACTGGATTTGTTCAG
TACGTTGAGAAGGGCGACCATGTCACACAAGGGCAAGAATTAATTGAATTCTGGCAACCA
GCAATTGAAAAGGCTGGCTTGGATGATACCGTTATGGTCGTTATTACAAACTATAAAGAT
ATGCCCGAATTTACGTATCTTAAAGATTCAGGAACCACCGTACAAGATGATGAAGATGTC
TTACAATTATCGAATGTCGGCTTACCGGATCAAGCATAG
> SEQ ID NO: 10 PlacLM of Lactiplantibacillus plantarum
DSM33967
GAAAAATCCCTCCGTAAATAGTTTTACTAAAATTTTATTAGTTTGACTATAATGCTTTTT
TTGATTGTTGTCAACACCCCATAAGACCTTTAGAACAAGATTTGGTAACGCTTTACAAAT
TTATCACGTTATCGATTCAAATTCTTCTTATCGCCCGTTGTCGTTGTCAGTAGTTGTTGT
CATTTAGTTAAAATTTAACTAAAACGACATATACAAATTTAATATTTTGTTTTATGATAT
TTGTAAGCGTTTTATTTATGTAACTTTGAAAAAAGCTTCCTC
> SEQ ID NO: 11 PlacLM of Lactiplantibacillus plantarum
DSM33121 (wild type)
GAAAAATCCCTCCGTAAATAGTTTTACTAAAATTTTATTAGTTTGACTATAATGCTTTTT
TTGATTGTTGTCAACACCCCATAAGACCTTTAGAACAAGATTTGGTAACGCTTTACAAAT
TTATCACGTTATCGATTCAAATTCTTCTTATCGCCCGTTGTCGTTGTCAGTAGTTGTTGT
CATTTAGTTAAAATTTAACTAAAACGACATATACAAATTTAATATTTTGTTTTATGATAT
TTGTAAGCGTTTTATTTATGTAACTTTGAAAGGAGCTTCCTC

1. A method of producing a fermented dairy product for storage at ambient temperature, comprising inoculating an initial fermented dairy product with one or more lactose-deficient bacterial strains, wherein the initial fermented food product comprises between about 0% and about 1% galactose.

2. The method of claim 1, wherein the one or more lactose-deficient bacterial strains comprise a beta-galactosidase protein having an amino acid sequence with at least about 70%, such as 75%, 80%, 85%, 90%, 95%, 96, 97%, 98%, 99%, 99.5%, or 100% sequence identity to the amino acid sequence encoded by the sequence of nucleotides set forth by SEQ ID NO:1.

3. The method of claim 1-2, wherein the one or more lactose-deficient bacterial strains comprise one or more mutation in the sequence of nucleotides of the promoter region of the beta-galactosidase gene lacL/M (PlacLM), which promoter region is defined by any one of SEQ ID NO:10 or SEQ ID NO:11.

4. The method of claim 3, wherein the mutation is in the −10 region of the promoter, such as a two-nucleotide mutation GG to AA at position 272-273 of SEQ ID NO:11.

5. The method of any one of claims 1-4, wherein the one or more lactose-deficient bacterial strains comprise or consist of a lactose-deficient L. plantarum strain.

6. A culture or kit of parts comprising a lactose-deficient bacterial strain, wherein the lactose deficient bacterial strain has a lactose metabolic activity of about or less than about 50%, 40%, 30%, 20%, or 10% or no lactose metabolic activity compared to a lactose metabolic activity of a lactose-metabolizing bacteria comprising a beta-galactosidase protein having the amino acid sequence encoded by the sequence of nucleotides set forth by SEQ ID NO:1.

7. The culture or kit of parts of claim 6, wherein the one or more lactose-deficient bacterial strains comprise a beta-galactosidase protein that has one or more amino acid substitution, addition or deletion compared to the amino acid sequence encoded by the sequence of nucleotides set forth by SEQ ID NO:7 and 8, or 1.

8. The culture or kit of parts of claims 6-7, wherein the one or more lactose-deficient bacterial strains comprise one or more mutation in the sequence of nucleotides of the promoter region of the beta-galactosidase gene lacL/M (PlacLM), which promoter region is defined by any one of SEQ ID NO:10 or SEQ ID NO:11.

9. The culture or kit of parts of claim 8, wherein the mutation is in the −10 region of the promoter, such as a two-nucleotide mutation GG to AA at position 272-273 of SEQ ID NO:11.

10. The culture or kit of parts of claims 6-9, wherein the one or more lactose-deficient bacterial strains comprise a sequence of nucleotides of the promoter region of the beta-galactosidase gene lacL/M (PlacLM) defined by SEQ ID NO:10, or a sequence of nucleotides with at least about 70%, such as 75%, 80%, 85%, 90%, 95%, 96, 97%, 98%, 99%, 99.5%, or 100% sequence identity to the sequence of nucleotides defined by SEQ ID NO:10, or a sequence of nucleotides with at least about 70%, such as 75%, 80%, 85%, 90%, 95%, 96,97%, 98%, 99%, 99.5%, or 100% sequence identity to the sequence of nucleotides defined by position 261-281 or of position 269-279 of SEQ ID NO:10.

11. The culture or kit of parts of any one of claims 6-10, wherein the lactose-deficient bacterial strain is a lactose-deficient L. plantarum strain.

12. The kit of parts of any one of claims 6-11, further comprising a galactose-metabolizing Streptococcus thermophilus strain.

13. Use of a culture of any one of claims 6-11 or kit of parts of any one of claims 6-12 to produce a fermented dairy product for storage at ambient temperature.

14. A fermented dairy product for storage at ambient temperature obtained by the method of any one of claims 1-5 or the use of claim 13.

15. A method for manufacturing a lactose-deficient bacterial strain comprising:
a) providing a lactic acid bacterial strain as the mother strain, such as Lactiplantibacillus plantarum DSM 33121;
b) introducing one or more mutations in the sequence of nucleotides of the promoter region of the beta-galactosidase gene lacL/M (PlacLM), such as the promoter region defined by any one of SEQ ID NO:10 or SEQ ID NO:11 of the strain; and
c) screening for a lactose-deficient bacterial strain have a reduced or complete inability to metabolize lactose.


SEQUENCE LISTING (11 sequences)

SEQ ID NO: 1 - DNA, 12318 bp, Lactobacillus plantarum
  source 1..12318: mol_type = genomic DNA
tcatttggtgctttcacgttctaccaacgtcgttcccagttcaatgcgctgggctgcttgacgatcatcttggaatcggctcaccaacaaatcgatggccgtctttcccatcagttcagtctcaacattgaccgagctaatctctggatacacatattcggccagcgtcgtgttattgaaactaaacaacttgacccgctgtggcacagcaatatcagcttgctgtaacgctttgagcgcaccggctgccatcggatcattagcgataaagaaagcgtgtggtaagcggtcacctaacatttcaatggcccgtttcatcatcagataaccagattgactagtgtaatcaccagctaatacatactcagcatgataggcatgatgccgctgcaacgcctgttcaaagctttgctgacgttgattgggaatctccacctgctgatcagtggaccactctttgccataaatcataccgatatctcggataccacgccgccaaaaataatcaacgacgcggttagtagcaaacgtaaaatcagtcaccacactgctgaatccctgagcaaagtgatcgtcatcaacaaaaacaagctggcttgacaaacgactcaactgttgtacttggacagggctaaattttcccaccgccacgattgcatcaatatcatcgccaacctgatctaaattattctggaagatactagtcgtcgtaaaaccacacacttgagcttgatgctcaattcctttacgaatcgctaaataatataagtcatcatgttcttgaatcttagagtaccactgaatcacagcgatatttttccgcattagcgtctttatatgctgacgtttacgcttactgtaccgcagctcgccagcaatcgtaaacacgcgcttgcgtgtctgctcgctcactgatagtgtcttgtcataatttaagacccgcgataccgtcgccaatgagacatctgctcgttgcgcaatatcttttaacgttacggccatcgtcattcctcttttctcttaattaattagcactgaataacaaacgtggggcaactgggcgtgacactattcaccaatcatgactcatcggtaaatcgcgtctgcaacaccccgttgccccacgtcttcatttgattaacaacttaatcgtgcattgctgcgacaaaacgatcaaaggctgcctcaccagcgtcatcccacttaaagaccccagcatcggccaggacccgggcaaagacgttgccgaccgccgtatcaatgacagtcgtcacattgtcagcggtgatggtattagtggccttcaattgatcagcccatgcttgatgcatcgcgaccatttgattaggctgatccaacaagtatttgccgacttcagctaattcggtcttcaaccgggctggtaaaatggcccggcccatcacttcaatcaagccaatattttccttcttgatatgttgaacatcctgatgtggatggaagatgccatctggaaattcagcagacgtctgattatcacgcaaaacgatatctaatacgtagtcatcgccaactttgcgtgcaatcggtgtcgttgtgtgatgacgtgtgccatccgtataagcacgaacatccacactctcgtccgaataattcttccacgtctcatgaatcttcacagcggcatccgttaatgcaaccaagtcctgactcgtcagccgaatcgtcgacattggccatttaacaatcccagccttaacacccgcgatacctaagtcaatcgtgcgtgcaattggcgctttcatcattggaaattcgtgccgaccaccttggtaatgctcgtgtgacagcattgaaccaccaacaattggcaagtccgcattactgcccacaaagtaatgtggaaattgctgaacaatttcaagtaagttggtaaaagtttgccggttaatgaccattggccggtgctcttgatccaagaaaatcgagtgttcattaaagtaagcatatggagaatattggaaaccccaagtattgccaccaagtgttaaccgaataattcggtgattgctccgtgctgggtaaccgagccggcctagatagccttcattctgcatacatagttggcaaagtggatagccatcttgcggttggttacgcgctgccgcaatcgctttaggatccttttcaggctttgataagttgatggtaatctctaagtcaccaaatggcgtttttgctggaaagaccacgtttttcgcaatggcccgcgtcttgatataatcattagctttgcttaagttaaagaaatagtcggttgccgtttgcggactaacctgatacttatcccaaaaacgttggtttaatacggatggtaacggggtcacaagatccatcaattgatccgctaagatatcttcatccgattgggtcgcttcgatcttgccattttgaatcgccgttttaaccaaggcatcggttaaacttgttaattggtcatcgcttgccgcaatcggatcaccctcgccgaccaaaccaagaacccggttatgcacatagattcgatctaattcagtatattgcgaaggcgacgcgatcaccgccgtgacaaattgatccaaactcgtcattatgcttggcctccccgatcatcatagccggccggatgactttctttccaagtccaagccgtcttgataatttctttcacatcatcatactgaggtgtccaacctaaaacagtacgtgccttgtcgctagctgcaaccaatgtgcttggatcaccagcgcggcgtggtgccatgattgctggaattggcttaccagtgacttcacgagcagcgtccaacatttgtttgttggagaagcccgttgaagaacccaagttaaacgcatcactatcgtgcccctgctttaggtattccaaggctaagatatgtgcatcagctaaatcgacaacgtgaacatagtcccgaacattggtcccgtctggagtgggatagtcgtcaccgaagatttgtaattgatcccgttcaccagcagctacttgtaaaataattgggacgagatgcgtttctggtgcgtgatcttcgccgatgctgccgtcaggcttagcccccgccacgttaaagtaacgtaaggccacgaatttaatcccgtaagccacatctgaccagtgcatgatcttttccatcgctaatttactttcaccgtatgggttagttggcacttgtgggtccgattccttgattggcacctgcttaggttcaccataagttgctgcggtcgatgagaagacgattttcttcacatcgtgcttagccaccacttcgagcaacttaaccatccctgccgtgttgttatcaaagtacttgagcggatccttcatcgattcaggaacaactgagaaagctgcaaaatgaatgacaccttcaacgttttcttggtcaaacacggtattcatgaacgccgtatcccggacatcaccttcataaaaacgcgcgtgttcattgaccgctgcccggtgccccgtgaccaagttatcaacaaccgccacgtcatacccctttgcaaccaaccgatcaactgcgtgtgaaccgatgtagccggcaccacctaaaactaaaactgccattctaattcctcctcaatatttctcaatgcttactgctaatttgtattttaaatcaattcttttattaaagttccttagggccgtctgcgatttcagcaatgtagaaatcagcgtcgtaaccaatcgtgtcgcgataaactttaccgacgttttccttaaaggcatcgacttggtccttatcaacgatggcaataccacagccaccaaagccagcaccagtcatccgcgcaccaagcacgcctggttgtttccaagctgtttcggccaacgtatccaattctttcccagtcacttcatagtcaaagtgtagtgaaacgtgggaagccgtcactaattcgccaaaggtcgttaaatcatcgtcagccaaggccttagttgcgcgaatggcccgttgattttcgaaaacggcatgccgggcacgtttgattaaggtttcatcattgattaagtaggccgcttcgtcaaactggtcgttagttaaatcgccaagtgatttgatgtctaacttagtttgtaatcggcgtaatgcttcttcacattctgagcggcgttcattatattttgaatccgccaattcgcgccgtttgttcgtattcatgataacgattacgttgttgcctaactttacgggtgcataagaatagtccatggtgttggtatcaagtaaaattgcttggtcctttttacccattccaacagcaaattgatccatgatcccggagttaacaccaacatagttgttttcgcacttttgaccaatcttaacaagatctaattggctaataccgagattaaaaccagtgtttaacatgattccagttaataactcaattgacgcagatgatgataatccagcaccatcaggcatgttgccatggacgtagaaatcaaaaccatgatcgaatttaacgccggtcttcgcgatttcgtccatcattcccttaggataatttgtccaaccagcggctttgtcatatgaaaggtcgttgacatcaaacgtgacgatcccagcatctggaatattagctgagtacatccgcactgtcgtatctgtccggggtgcataaacaccatatgtcccgattgtaatcgcacatgggaaaacatggcccccgttataatcagtatgttcaccgatcaagttgattcgaccaggtgagaaaaagacccgttctggcttggtatcaaaggcttcggtaaactcctgttttaagtcgctagtattcatgaaaaatccctccgtaaatagttttactaaaattttattagtttgactataatgctttttttgattgttgtcaacaccccataagacctttagaacaagatttggtaacgctttacaaatttatcacgttatcgattcaaattcttcttatcgcccgttgtcgttgtcagtagttgttgtcatttagttaaaatttaactaaaacgacatatacaaatttaatattttgttttatgatatttgtaagcgttttatttatgtaactttgaaaaaagcttcctcatgcaagctaatcttcaatggttagatgacccagaagtcttccgggtcaaccaattacctgcacatagtgatcaccattattatcacgacacagcagaattcaaaacgggtagtcgcttcatcaagagtctcaatggcgcttggcgttttaacttcgccaagacaccggctgaacgcccagttgatttttatcaacccgatttcgatgcaaccgactttgatacgattcaggttcccggtcatattgaactagccggctatggtcaaattcaatacattaacacgctatacccatgggaaggtaaaatttaccgtcgcccaccgtataccctcaatcaagatcaattaacaccaggcctattcagtgacgctgcggacaacaccgtcggctcgtacctcaaaaccttcgatctcgacgatgcttttaaagggcaacgtattatcattcagttccaaggggtagaagaagccctgtacgtctggttaaatggccattttattggctacgctgaagatagtttcaccccttctgaatttgatttgacgccgtatattcaggaccaaggtaacgttttagcggttcgggtctacaaacgcagtactgctgcctttattgaagaccaagatatgttccgtttctctggtattttccgtgacgtcaatatactggcggagcctgctagccatattactgatttggacatccgaccagttccaaatgccaatctcaaaagtggtgagctcaacatcactactaaagtaaccggcgaaccagccactttagcgctgaccgttaaagaccatgacgggcgagtactgacgagtcaaacgcaaaccggtagtggcagtgtaacctttgataccatgttattcgaccaactgcacttgtggtcaccacaaacgccgtatctctatcaattgacaattgaagtttacgatgctgatcaccaactcttggaagtcgtcccatatcagtttgggttccggacggtcgagctgcgcgatgacaaagtcatttacgtcaacaataaacggttggtgatcaacggggttaaccggcacgaatggaacgcccacaccggtcgcgttatcagtatggctgatatgcgcgctgatatccaaaccatgttagctaacaatatcaatgccgatcggacctgccattatcctgatcaattaccttggtatcaattatgtgacgaggccggtatctacctaatggccgaaaacaacctcgaatcgcacgggtcatggcaaaagataggggctatcgagccttcttacaatgttcctggcgataatccacactggttagcagcggtgatcgaccgggcccgttcaaactacgaatggtttaaaaaccacccatcgatcattttttggtcacttggcaatgaatcgtatgctggcgaagatatcgcggcgatgcaggctttttataaagaacacgatgattcacgactcgtccactacgaaggtgttgtccacacaccagaattaaaagatcgcatttctgatgttgaaagtcggatgtacgaaaaaccccaaaatattgtagcttacttggaagataacccaaccaaacctttcctagattgtgaatatatgcatgacatggggaattctctgggcggtatgcaatcatataatgatttgatcgacaagtatccgatgtatcaaggtggctttatttgggactttattgatcaagccctcttcgttcatgacccaattaccgaccaagacgtgctccggtatggcggtgatttcgacgaacgccactccgattatgaattctccggtgacggcttaatgtttgctgaccggacgccaaaaccagcaatgcaagaggtgaaatattattatggcttacacaaataatcaactacacgttatttacggcgacgggagtttaggactacagggggctaatttccactacctctttagctacgaacgtggcggacttgaatcactcgtcgtcaacgataaagaatggctctatcgtacacccacgcccatcttttggcgggcgacaaccgataatgatcacggtagcggcttttcagtcaaatccgcacagtggtacgcggccgataagttctcaacttgtcaagatatcgaattgacggttgacgaccaaccagtcacaccgttaccaatcgcgccactcaataacaaatacacggatcacgaaatcgccacgaaagtctcactggcttaccacttcgttaccacgaccgttcctagtaccatcgtcacagtgacttatacggtgacagcagacggtcagatcaatatcgccacccattatagcggtcagtctgatttgccagagctacccgcatttggtctgcggtttatcataccaactaccgcgaccggcttcgactataccggtttgtccggtgagacttatcctgaccggctggccggcgcaacgcacgggcgattccacgttgacagtctgccagtcacaccatacttggtcccacaagaatgcggcatgcacatgcaaactgaacaagtgacagtaacgcgatcaacaacacaaaataacgctgaccacgacaacacaccgttcagtttgacatttagccaagccgatgcaccattcgccttcagctgccttccctataccgccgctgaactagaaaacgcaacgcacatggaagaattaccattagcacggcgaacggtcttatcaatctacggtgccgttcgtggggtcggtggcattgatagttggggaacagacgtagaatccccatatcatatccccgctgatcaagacattgacttcagctttaatattcatttctaaaagttattttgatttcaaaagaacgctccggcgagttatttgccagagcgttcttttagattaacgatgattaagttttaatatgtttaatggctgagcttagtccttagccttgaagtaatgtacctgcgccgtaaagtcactagtttgaacgggagtcgtgtagagtcctagttgcatcaattcatcgccaccgtacattttgtttgtgtcggtctcaacgtaggtctgctgagggtctaatcccgctaacttcgtaatatgcggttctggttgaactgcacctaaaataacgaaggtgaacagcaaggcttctttctgatccggactaacaaacatccacgccaccgtattagattcaaacgggctctctaatcgataaaaggtcccgtattgaactaactcacggtgctgcttatagaaggcaacctgtcttttaacggcctgcttgtccgcatcacttagttgggccgcgtccagttcatagcccaaggtaccactcattgccacagcaccacgcatcttcatcgacgtcgaccgtcctaacaattcatccgggctcgtcccaacatgggcggtaattgcagaaattggataaacgagtgaagtcccatattgaattttgagccgttcaatcgggtcattattatctgatggccaactctgtggcatataatacattaaaccagcatcgaagcggccaccaccgccagagcagccttcaaataagatctgtggatagcgttttgttaaacgctccatcaagtcatatacacctaagataaagcggtggctagtctcaccttcatgagttgaatcgctatgtggcgaatagacttccgttaaattgcggttcatatcccacttgatataatcaatgggcaccttatcaagaactgcggtcatttgttcaaaaatattgtcgacaacatccggctggctgaaatcaagcacgtattcgttccgcgataacgttcggccacgatctggtacgcctaacacccaatcaggatgggcccgataaagatcggaatcggctgaaatattttctggctcgaaccaaagtccgaaccgcatccctttatcatgggtccgtttgctaatatccgctaagcctcctggtaacttgttccgattaacaaaccagtcacctagcgaactattatcatcatttcgatgaccgaaccaaccgtcatccagcacaaacatttcgatacccagtggtgctgcagcatccaagattgattgaatcttgtccgcatcaaagtcaaagtaagtggcttcccaattgttgatcaagatcggccggagatcgtgcttgtaacgactgcgcgccacgcgatcacgcaataaatcatggtaggtttgcgacataccattaagccccgtcgtcgagtataccattgcaacctcgggcgtttggaaactatcaccggccgccaatcgccagtcgaagttatattcattgattcccatagtcaagcgtgtctgaccaattgggtcccgttctaaactaatctggtggttacctgaataaattagcaatgcaccaaggacattgccttgaaattcattagtattcggatcagctagcgccacaaaaggattcatatgatgagaactagcaccccggcgactttcaaactgagttacacctcgatggagccgttcacgactaagctggcgctcacgcgcatagcttccaggtaacgaaatggcatctaatggttgggttggcaggtccatctgcatcgaagccactttttctaagtcgacagcttgctcactgtgattaatcaaccgggcgctacgcgtgataactggccgatcccgataaatcgtgtaggctaattcgagtgtcacgcctaatgtggcatctcgcaacgtcacaatcaaagtttgggcttcatcctcatcttcgacataagtttgtggcaagcccttaagttccggcttaccggcttccatccggcagtcttgataacgaaaatcaactgttcgagcaccattggccgcgcgaataatcgcggcgggttgccgataatcaccggtattattgccaccgtattcctgtggcaaatcatccttggaatagcttcgatcagtcgtattcggtaaatttcctgagaacccccgatcaatccggggatactgacgctcgccatgataaccccgaacccgtgggccaaagtacaagtgggccaacacgttgcctgtctccacacctagaatatacgagatttgatcgttatgcaggtgaaagacaagttgttcttcatcaatgtcaattaaagtttgctgcaacgttactgccatagtaaaatcttcccttcgctattagctttttctatttcagttcacaaaaagagctattaatggctcgcgtcattaatagctctcctcgtaaagaaactatgcttgatccggtaagccgacattcgataattgtaagacatcttcatcatcttgtacggtggttcctgaatctttaagatacgtaaattcgggcatatctttatagtttgtaataacgaccataacggtatcatccaagccagccttttcaattgctggttgccagaattcaattaattcttgcccttgtgtgacatggtcgcccttctcaacgtactgaacaaatccagtcccacgcatcttaacggtaccaatcccaatatgaattagcgttaaaataccggtatcagaacgtaatccgattgcatggcgagtcgggaaggttgcaacaacttcaccatcaaatggtgcgacaacgcgaccatccgttggtttaatggcgaaacctttacccatactaccgtttgcgaaggattcgtcagcaacatctttcaaactgatcagttccccggcaacaggatcttgataaaccgtcgtcttggcatcggcttctgcttcaacttcagcaggagtttcatcggtttccaagtgagttgaccatgttttttcaagttcatcaacgatttgtgcatgcatcttttcatccaacttaaccttcttgaagaagatgaatgtcccaatcaaaatcatagcagcaggtacaccgaacatcattaacttgaagatggcagcgccatggctagtcacatcaccagcagtggcaccagctgtcatcccagcccaaacagcagttaaaccaacgacaccgttagaaatcgcaccgcctaatttatcaagtaaaggccgaacggataaggttaaagattcgtcacgatgaccaaacttcaactgcccatattcaacagaatcactcaaaaccatcaaaacaactaagaaaattaatggttgtggaatgtagaataatacggccgctgttaagatcattggtaatgactgaccagcaaaggcaaacatgatcagtgaaatcatcataataacaactgctaagaagaacactttacgacgactaaacttctttgctaatggtgggaaggccaatacggcaaaaatcccaataatcgcatttagcgaacctaatagtgtaaacttcgaggcgtcacccaaaatataggtgaagtagtaaagttctagtgagttcgtaatggcaatccctgccgtgtagatcccatacatcaatgataaccacatcaattgatcgttacgggctaacactttgaagacttccttgaagctagtcttttcagtgttcttacgcaattcagaatcattttctttggttccaagtgcaacaactaaggcagaaatcaaagcgattgcggcaataatgatcccaaatgccatccaaccacggttgtcaccttgaccactgttagacttaacagaaaacattaagacgattggcataacgataaccccaacgatgttggcaccaatgttagagccaacccgggcgtatgtcgccgtcttttcacgttctgctgagtcaaatgagattgctggaatcattgaccaaaaaccaacatctttaaacgaatagaaaatatccattgtaatgtataaaattgcaaaaataattaaatatagaatggggtttgaggtatttaatccgcctaaactcgtgaataaaagtgctaaaacgacggaacctataatcccaccaatgacaatccatggtttaaagtggccccaccgagaattagtattatcaatggcattcccgatcaaaggatcaattgctagttccacgaaccgcaaacctgcaataatcatggtgatccaagcaatcatttgtgttccttgagccccactactcttatcgaacaagtgggaggtcacaaacataataaaataagttgataaagtggcatagaaggcatcgtgcccaaacgccccaaacgaataagcaagtcgcgatgttattttacgacctttactctgctttgtctctgcatccat

SEQ ID NO: 2 - DNA, 2067 bp, Lactobacillus plantarum
  source 1..2067: mol_type = genomic DNA
atgaataagttagaactggttaaaggctttttacatggtggtgactacaatcctgatcagtggttaaaacgaccggatattttggaccgggattatcagttgatggatcagtcgaagattaacacggttactgttggtgtttttgcttggggacggcttgaaccggaagaaggtgtttatgattttgcatggcttgatgatgttttcgataaaatgaatagtcgaaatggccacgtgattttagcaacaccaagtggcgcacggccacgatggatgagtcagaaatatcctgaggttaatcgagttgattctgaaggtcgtcgtcatactcatggtttccgacataatcattgctattcgtccccgatttatcgaagaaaggtacatgaaatcaatatgcgcttggcagagcgatatggagacaattcagcgcttgttttgtggcatgtctctaatgaatattctggagagtgttactgccctttgtgtcaacaaaagtggcgggagtgggtccagcacaaatataagacacttgaagcattagatgacgcgtggtgctcgagcgtctggagtggtttatacagtgattggtcacaattgttgccaccatcactattgggtgatagcaaagttcatggcatggatttagactggcaccggtttgtgtcagatatgacgattgatttttatcagcatgagattcagcctttaaagaaagtaacgcccaatattcccgtaacaactaattttatggctgagagcttaaaagaagggcagtatacaccgctgacagggttagattatagtagatttgcaaagtctttagatataattagttgggatagttatcctagttggcataatcgctatgaaattactgcggaaactgccatgaaaaccgcgtatgttcatgatcagttttggtctttaaaacaacaaccatttttggtcatggagtcaacaccaagctgtgttaactggggacagtataataaagccaagcaacctggcatgcatcggctgagttcatttcaacagattgcgcatggctcagatagtacgttatacttccagattagacaggcacaaggtaattcagaaaagtatcatggtgccgtaattggacatgatgatagctcagatagccgcgtttttaaagacgtgcaacaatatggacaagatttggaaaaaattgtagaagttaaaggggctattaagcgtgtaagagttgctatcctctttgactgggatagcaactgggccctgaaccgtggtggcggctttggtcggccaactagattgggaatacaagtacttcaaaaacattatgcaaatttttggcacaaagatattgagtgtaatgttataacgagtgatatggacttttctcaatatgatttgttaattgcaccgatgctttatttaatacgacaagatacgatgaaaaaattgcaaaaatttgttcaaagtggtggcacattggtatctagctatttttcaggtatggttgatagcaatgatcgtgtgaatttagatggctggcctaaaccgctacaggatatttttggaattgagccaaaagaattggatactttattaccaggcgagcactaccatgttagttttaatcaaaaagagtatgaaactaccgattatgatcaaataatgaaagttaaaaacgcatcagtgctgggatactacacagacaacttctatgttggaacaccagcaattacacggcatcagtatggtaaaggaacggcctattatattggtgcacgcttgtctgcggaattcattgaagacttttatgatgacattattcaaagccttaatttagataatagttttgtcgaattgagcaatccggtagtatcagttcaaactcgttataatgaggattatgcctatcactttgttatgaatttttctgaacaagtacaaatggttaattttcgacgtccatcctttgatctgcttacacagcgagaagttacgaaatcagtgcaaattgataagtatggggtcatgattctgaaggaaagacagaacaatacgttgcactag

SEQ ID NO: 3 - DNA, 1002 bp, Lactobacillus plantarum
  source 1..1002: mol_type = genomic DNA
atggccgtaacgttaaaagatattgcgcaacgagcagatgtctcattggcgacggtatcgcgggtcttaaattatgacaagacactatcagtgagcgagcagacacgcaagcgcgtgtttacgattgctggcgagctgcggtacagtaagcgtaaacgtcagcatataaagacgctaatgcggaaaaatatcgctgtgattcagtggtactctaagattcaagaacatgatgacttatattatttagcgattcgtaaaggaattgagcatcaagctcaagtgtgtggttttacgacgactagtatcttccagaataatttagatcaggttggcgatgatattgatgcaatcgtggcggtgggaaaatttagccctgtccaagtacaacagttgagtcgtttgtcaagccagcttgtttttgttgatgacgatcactttgctcagggattcagcagtgtggtgactgattttacgtttgctactaaccgcgtcgttgattatttttggcggcgtggtatccgagatatcggtatgatttatggcaaagagtggtccactgatcagcaggtggagattcccaatcaacgtcagcaaagctttgaacaggcgttgcagcggcatcatgcctatcatgctgagtatgtattagctggtgattacactagtcaatctggttatctgatgatgaaacgggccattgaaatgttaggtgaccgcttaccacacgctttctttatcgctaatgatccgatggcagccggtgcgctcaaagcgttacagcaagctgatattgctgtgccacagcgggtcaagttgtttagtttcaataacacgacgctggccgaatatgtgtatccagagattagctcggtcaatgttgagactgaactgatgggaaagacggccatcgatttgttggtgagccgattccaagatgatcgtcaagcagcccagcgcattgaactgggaacgacgttggtagaacgtgaaagcaccaaatga

SEQ ID NO: 4 - DNA, 1464 bp, Lactobacillus plantarum
  source 1..1464: mol_type = genomic DNA
atgacgagtttggatcaatttgtcacggcggtgatcgcgtcgccttcgcaatatactgaattagatcgaatctatgtgcataaccgggttcttggtttggtcggcgagggtgatccgattgcggcaagcgatgaccaattaacaagtttaaccgatgccttggttaaaacggcgattcaaaatggcaagatcgaagcgacccaatcggatgaagatatcttagcggatcaattgatggatcttgtgaccccgttaccatccgtattaaaccaacgtttttgggataagtatcaggttagtccgcaaacggcaaccgactatttctttaacttaagcaaagctaatgattatatcaagacgcgggccattgcgaaaaacgtggtctttccagcaaaaacgccatttggtgacttagagattaccatcaacttatcaaagcctgaaaaggatcctaaagcgattgcggcagcgcgtaaccaaccgcaagatggctatccactttgccaactatgtatgcagaatgaaggctatctaggccggctcggttacccagcacggagcaatcaccgaattattcggttaacacttggtggcaatacttggggtttccaatattctccatatgcttactttaatgaacactcgattttcttggatcaagagcaccggccaatggtcattaaccggcaaacttttaccaacttacttgaaattgttcagcaatttccacattactttgtgggcagtaatgcggacttgccaattgttggtggttcaatgctgtcacacgagcattaccaaggtggtcggcacgaatttccaatgatgaaagcgccaattgcacgcacgattgacttaggtatcgcgggtgttaaggctgggattgttaaatggccaatgtcgacgattcggctgacgagtcaggacttggttgcattaacggatgccgctgtgaagattcatgagacgtggaagaattattcggacgagagtgtggatgttcgtgcttatacggatggcacacgtcatcacacaacgacaccgattgcacgcaaagttggcgatgactacgtattagatatcgttttgcgtgataatcagacgtctgctgaatttccagatggcatcttccatccacatcaggatgttcaacatatcaagaaggaaaatattggcttgattgaagtgatgggccgggccattttaccagcccggttgaagaccgaattagctgaagtcggcaaatacttgttggatcagcctaatcaaatggtcgcgatgcatcaagcatgggctgatcaattgaaggccactaataccatcaccgctgacaatgtgacgactgtcattgatacggcggtcggcaacgtctttgcccgggtcctggccgatgctggggtctttaagtgggatgacgctggtgaggcagcctttgatcgttttgtcgcagcaatgcacgattaa

SEQ ID NO: 5 - DNA, 1005 bp, Lactobacillus plantarum
  source 1..1005: mol_type = genomic DNA
atggcagttttagttttaggtggtgccggctacatcggttcacacgcagttgatcggttggttgcaaaggggtatgacgtggcggttgttgataacttggtcacggggcaccgggcagcggtcaatgaacacgcgcgtttttatgaaggtgatgtccgggatacggcgttcatgaataccgtgtttgaccaagaaaacgttgaaggtgtcattcattttgcagctttctcagttgttcctgaatcgatgaaggatccgctcaagtactttgataacaacacggcagggatggttaagttgctcgaagtggtggctaagcacgatgtgaagaaaatcgtcttctcatcgaccgcagcaacttatggtgaacctaagcaggtgccaatcaaggaatcggacccacaagtgccaactaacccatacggtgaaagtaaattagcgatggaaaagatcatgcactggtcagatgtggcttacgggattaaattcgtggccttacgttactttaacgtggcgggggctaagcctgacggcagcatcggcgaagatcacgcaccagaaacgcatctcgtcccaattattttacaagtagctgctggtgaacgggatcaattacaaatcttcggtgacgactatcccactccagacgggaccaatgttcgggactatgttcacgttgtcgatttagctgatgcacatatcttagccttggaatacctaaagcaggggcacgatagtgatgcgtttaacttgggttcttcaacgggcttctccaacaaacaaatgttggacgctgctcgtgaagtcactggtaagccaattccagcaatcatggcaccacgccgcgctggtgatccaagcacattggttgcagctagcgacaaggcacgtactgttttaggttggacacctcagtatgatgatgtgaaagaaattatcaagacggcttggacttggaaagaaagtcatccggccggctatgatgatcggggaggccaagcataa

SEQ ID NO: 6 - DNA, 1164 bp, Lactobacillus plantarum
  source 1..1164: mol_type = genomic DNA
atgaatactagcgacttaaaacaggagtttaccgaagcctttgataccaagccagaacgggtctttttctcacctggtcgaatcaacttgatcggtgaacatactgattataacgggggccatgttttcccatgtgcgattacaatcgggacatatggtgtttatgcaccccggacagatacgacagtgcggatgtactcagctaatattccagatgctgggatcgtcacgtttgatgtcaacgacctttcatatgacaaagccgctggttggacaaattatcctaagggaatgatggacgaaatcgcgaagaccggcgttaaattcgatcatggttttgatttctacgtccatggcaacatgcctgatggtgctggattatcatcatctgcgtcaattgagttattaactggaatcatgttaaacactggttttaatctcggtattagccaattagatcttgttaagattggtcaaaagtgcgaaaacaactatgttggtgttaactccgggatcatggatcaatttgctgttggaatgggtaaaaaggaccaagcaattttacttgataccaacaccatggactattcttatgcacccgtaaagttaggcaacaacgtaatcgttatcatgaatacgaacaaacggcgcgaattggcggattcaaaatataatgaacgccgctcagaatgtgaagaagcattacgccgattacaaactaagttagacatcaaatcacttggcgatttaactaacgaccagtttgacgaagcggcctacttaatcaatgatgaaaccttaatcaaacgtgcccggcatgccgttttcgaaaatcaacgggccattcgcgcaactaaggccttggctgacgatgatttaacgacctttggcgaattagtgacggcttcccacgtttcactacactttgactatgaagtgactgggaaagaattggatacgttggccgaaacagcttggaaacaaccaggcgtgcttggtgcgcggatgactggtgctggctttggtggctgtggtattgccatcgttgataaggaccaagtcgatgcctttaaggaaaacgtcggtaaagtttatcgcgacacgattggttacgacgctgatttctacattgctgaaatcgcagacggccctaaggaactttaa

SEQ ID NO: 7 - DNA, 1881 bp, Lactobacillus plantarum
  source 1..1881: mol_type = genomic DNA
atgcaagctaatcttcaatggttagatgacccagaagtcttccgggtcaaccaattacctgcacatagtgatcaccattattatcacgacacagcagaattcaaaacgggtagtcgcttcatcaagagtctcaatggcgcttggcgttttaacttcgccaagacaccggctgaacgcccagttgatttttatcaacccgatttcgatgcaaccgactttgatacgattcaggttcccggtcatattgaactagccggctatggtcaaattcaatacattaacacgctatacccatgggaaggtaaaatttaccgtcgcccaccgtataccctcaatcaagatcaattaacaccaggcctattcagtgacgctgcggacaacaccgtcggctcgtacctcaaaaccttcgatctcgacgatgcttttaaagggcaacgtattatcattcagttccaaggggtagaagaagccctgtacgtctggttaaatggccattttattggctacgctgaagatagtttcaccccttctgaatttgatttgacgccgtatattcaggaccaaggtaacgttttagcggttcgggtctacaaacgcagtactgctgcctttattgaagaccaagatatgttccgtttctctggtattttccgtgacgtcaatatactggcggagcctgctagccatattactgatttggacatccgaccagttccaaatgccaatctcaaaagtggtgagctcaacatcactactaaagtaaccggcgaaccagccactttagcgctgaccgttaaagaccatgacgggcgagtactgacgagtcaaacgcaaaccggtagtggcagtgtaacctttgataccatgttattcgaccaactgcacttgtggtcaccacaaacgccgtatctctatcaattgacaattgaagtttacgatgctgatcaccaactcttggaagtcgtcccatatcagtttgggttccggacggtcgagctgcgcgatgacaaagtcatttacgtcaacaataaacggttggtgatcaacggggttaaccggcacgaatggaacgcccacaccggtcgcgttatcagtatggctgatatgcgcgctgatatccaaaccatgttagctaacaatatcaatgccgatcggacctgccattatcctgatcaattaccttggtatcaattatgtgacgaggccggtatctacctaatggccgaaaacaacctcgaatcgcacgggtcatggcaaaagataggggctatcgagccttcttacaatgttcctggcgataatccacactggttagcagcggtgatcgaccgggcccgttcaaactacgaatggtttaaaaaccacccatcgatcattttttggtcacttggcaatgaatcgtatgctggcgaagatatcgcggcgatgcaggctttttataaagaacacgatgattcacgactcgtccactacgaaggtgttgtccacacaccagaattaaaagatcgcatttctgatgttgaaagtcggatgtacgaaaaaccccaaaatattgtagcttacttggaagataacccaaccaaacctttcctagattgtgaatatatgcatgacatggggaattctctgggcggtatgcaatcatataatgatttgatcgacaagtatccgatgtatcaaggtggctttatttgggactttattgatcaagccctcttcgttcatgacccaattaccgaccaagacgtgctccggtatggcggtgatttcgacgaacgccactccgattatgaattctccggtgacggcttaatgtttgctgaccggacgccaaaaccagcaatgcaagaggtgaaatattattatggcttacacaaataa

SEQ ID NO: 8 - DNA, 960 bp, Lactobacillus plantarum
  source 1..960: mol_type = genomic DNA
atggcttacacaaataatcaactacacgttatttacggcgacgggagtttaggactacagggggctaatttccactacctctttagctacgaacgtggcggacttgaatcactcgtcgtcaacgataaagaatggctctatcgtacacccacgcccatcttttggcgggcgacaaccgataatgatcacggtagcggcttttcagtcaaatccgcacagtggtacgcggccgataagttctcaacttgtcaagatatcgaattgacggttgacgaccaaccagtcacaccgttaccaatcgcgccactcaataacaaatacacggatcacgaaatcgccacgaaagtctcactggcttaccacttcgttaccacgaccgttcctagtaccatcgtcacagtgacttatacggtgacagcagacggtcagatcaatatcgccacccattatagcggtcagtctgatttgccagagctacccgcatttggtctgcggtttatcataccaactaccgcgaccggcttcgactataccggtttgtccggtgagacttatcctgaccggctggccggcgcaacgcacgggcgattccacgttgacagtctgccagtcacaccatacttggtcccacaagaatgcggcatgcacatgcaaactgaacaagtgacagtaacgcgatcaacaacacaaaataacgctgaccacgacaacacaccgttcagtttgacatttagccaagccgatgcaccattcgccttcagctgccttccctataccgccgctgaactagaaaacgcaacgcacatggaagaattaccattagcacggcgaacggtcttatcaatctacggtgccgttcgtggggtcggtggcattgatagttggggaacagacgtagaatccccatatcatatccccgctgatcaagacattgacttcagctttaatattcatttctaa

SEQ ID NO: 9 - DNA, 1959 bp, Lactobacillus plantarum
  source 1..1959: mol_type = genomic DNA
atggatgcagagacaaagcagagtaaaggtcgtaaaataacatcgcgacttgcttattcgtttggggcgtttgggcacgatgccttctatgccactttatcaacttattttattatgtttgtgacctcccacttgttcgataagagtagtggggctcaaggaacacaaatgattgcttggatcaccatgattattgcaggtttgcggttcgtggaactagcaattgatcctttgatcgggaatgccattgataatactaattctcggtggggccactttaaaccatggattgtcattggtgggattataggttccgtcgttttagcacttttattcacgagtttaggcggattaaatacctcaaaccccattctatatttaattatttttgcaattttatacattacaatggatattttctattcgtttaaagatgttggtttttggtcaatgattccagcaatctcatttgactcagcagaacgtgaaaagacggcgacatacgcccgggttggctctaacattggtgccaacatcgttggggttatcgttatgccaatcgtcttaatgttttctgttaagtctaacagtggtcaaggtgacaaccgtggttggatggcatttgggatcattattgccgcaatcgctttgatttctgccttagttgttgcacttggaaccaaagaaaatgattctgaattgcgtaagaacactgaaaagactagcttcaaggaagtcttcaaagtgttagcccgtaacgatcaattgatgtggttatcattgatgtatgggatctacacggcagggattgccattacgaactcactagaactttactacttcacctatattttgggtgacgcctcgaagtttacactattaggttcgctaaatgcgattattgggatttttgccgtattggccttcccaccattagcaaagaagtttagtcgtcgtaaagtgttcttcttagcagttgttattatgatgatttcactgatcatgtttgcctttgctggtcagtcattaccaatgatcttaacagcggccgtattattctacattccacaaccattaattttcttagttgttttgatggttttgagtgattctgttgaatatgggcagttgaagtttggtcatcgtgacgaatctttaaccttatccgttcggcctttacttgataaattaggcggtgcgatttctaacggtgtcgttggtttaactgctgtttgggctgggatgacagctggtgccactgctggtgatgtgactagccatggcgctgccatcttcaagttaatgatgttcggtgtacctgctgctatgattttgattgggacattcatcttcttcaagaaggttaagttggatgaaaagatgcatgcacaaatcgttgatgaacttgaaaaaacatggtcaactcacttggaaaccgatgaaactcctgctgaagttgaagcagaagccgatgccaagacgacggtttatcaagatcctgttgccggggaactgatcagtttgaaagatgttgctgacgaatccttcgcaaacggtagtatgggtaaaggtttcgccattaaaccaacggatggtcgcgttgtcgcaccatttgatggtgaagttgttgcaaccttcccgactcgccatgcaatcggattacgttctgataccggtattttaacgctaattcatattgggattggtaccgttaagatgcgtgggactggatttgttcagtacgttgagaagggcgaccatgtcacacaagggcaagaattaattgaattctggcaaccagcaattgaaaaggctggcttggatgataccgttatggtcgttattacaaactataaagatatgcccgaatttacgtatcttaaagattcaggaaccaccgtacaagatgatgaagatgtcttacaattatcgaatgtcggcttaccggatcaagcatag

SEQ ID NO: 10 - DNA, 282 bp, Lactobacillus plantarum
  source 1..282: mol_type = genomic DNA
gaaaaatccctccgtaaatagttttactaaaattttattagtttgactataatgctttttttgattgttgtcaacaccccataagacctttagaacaagatttggtaacgctttacaaatttatcacgttatcgattcaaattcttcttatcgcccgttgtcgttgtcagtagttgttgtcatttagttaaaatttaactaaaacgacatatacaaatttaatattttgttttatgatatttgtaagcgttttatttatgtaactttgaaaaaagcttcctc

SEQ ID NO: 11 - DNA, 282 bp, Lactobacillus plantarum
  source 1..282: mol_type = genomic DNA
gaaaaatccctccgtaaatagttttactaaaattttattagtttgactataatgctttttttgattgttgtcaacaccccataagacctttagaacaagatttggtaacgctttacaaatttatcacgttatcgattcaaattcttcttatcgcccgttgtcgttgtcagtagttgttgtcatttagttaaaatttaactaaaacgacatatacaaatttaatattttgttttatgatatttgtaagcgttttatttatgtaactttgaaaggagcttcctc